(19) United States
(12) Patent Application Publication
(10) Pub. No.: US 2025/0268938 A1
(43) Pub. Date: Aug. 28, 2025

(54) SMALL MOLECULE-REGULATED CELL SIGNALING EXPRESSION SYSTEM

(71) Applicant: Outpace Bio, Inc., Seattle, WA (US)

(72) Inventors: Glenna FOIGHT, Seattle, WA (US); Scott BOYKEN, Seattle, WA (US); Marc LAJOIE, Seattle, WA (US)

(21) Appl. No.: 18/274,944

(22) PCT Filed: Jan. 31, 2022
(86) PCT No.: PCT/US2022/014624

§ 371 Date: Jul. 28, 2023

Related U.S. Application Data
(60) Provisional application No. 63/143,725, filed on Jan. 29, 2021.

(51) Int. Cl. A61K 35/17; A61K 40/11; A61K 40/31; A61K 40/42; A61P 35/00; C07K 16/28

(52) U.S. Cl. CPC A61K 35/17; A61K 40/11; A61K 40/31; A61K 40/421; A61P 35/00; C07K 16/2803; A61K 2239/13; A61K 2239/21; A61K 2239/22; A61K 2239/24

(57) ABSTRACT

The disclosure provides a first fusion protein comprising a chimeric polypeptide (e.g., a chimeric antigen receptor) and a dimerization domain and a second fusion protein comprising a signaling domain and a dimerization domain, wherein the dimerization domains are capable of forming a dimer and wherein small molecule regulators (e.g., inducers and inhibitors) are capable of altering the state of the dimer to alter the activity of the CAR. Small molecule regulators, cells expressing the fusion proteins of the disclosure, and compositions comprising the proteins, small molecules and cells of the disclosure are provided. Methods of treating or preventing disease or disorders in a subject by providing a composition of the disclosure are described.

### RELATED APPLICATIONS

This application is a 35 U.S.C. § 371 national stage entry of PCT/US2022/014624, filed Jan. 31, 2022, titled the same, which claims priority to U.S. Provisional Application No. 63/143,725, filed Jan. 29, 2021. The entire contents of each of the prior applications are incorporated by reference herein.

### INCORPORATION OF THE SEQUENCE LISTING

This application contains a Sequence Listing that has been submitted in ASCII format via U.S. Postal Service Express Mail and is hereby incorporated by reference in its entirety. The ACSII file, created on Mar. 9, 2024 is named “OTPC_024_01US_SeqList_ST25.txt” and is 415,328 bytes in size.

### FIELD OF THE DISCLOSURE

The disclosure relates to small molecule-regulated cell signaling expression systems in the fields of small molecule chemistry, molecular biology, gene therapy, cellular therapy, immunology, and immune cell signaling. The expression systems of the disclosure use a modular design and changeable domains to target cell surface antigens and, upon receptor-ligand interactions, direct signaling cascades, thereby driving cellular functions.

### BACKGROUND

Small molecule drugs that control dimerization or protease activity can be used to control the activity of cell and gene therapies for improved efficacy and safety. To develop next-generation cell and gene therapies, including immunotherapeutic approaches against cancer, that are efficacious and safe, there is an increasing need for technologies that exploit modular designs and changeable domains for CARs that can target an array of cell surface antigens and, upon receptor-ligand interactions, direct signaling cascades.

### SUMMARY

The disclosure provides a fusion protein comprising a chimeric polypeptide operably-linked to a dimerization domain, wherein the chimeric polypeptide comprises an extracellular domain comprising an antigen sensing domain, a transmembrane domain, and an intracellular domain, and wherein the the chimeric polypeptide is operably linked to the dimerization domain intracellularly.

In some embodiments of the fusion proteins of the disclosure, the chimeric polypeptide comprises a T Cell Receptor (TCR), a Chimeric Antigen Receptor (CAR), or a chimeric receptor.

In some embodiments of the fusion proteins of the disclosure, the antigen sensing domain comprises one or more of (a) a set of three complementarity determining regions (CDRs) of a heavy chain variable region; (b) a set of three complementarity determining regions (CDRs) of a heavy chain variable region and a set of three complementarity determining regions (CDRs) of a light chain variable region; (c) a fibronectin-protein based scaffold; wherein the antigen sensing region specifically binds a target antigen. In some embodiments, the fibronectin-protein based scaffold comprises a Centyrin. In some embodiments, the fibronectin-protein based scaffold comprises a fibronectin type III (FN3) domain.

In some embodiments of the fusion proteins of the disclosure, the antigen sensing region comprises one or more sequences isolated or derived from a mammalian sequence. In some embodiments, the antigen sensing region comprises one or more sequences isolated or derived from a human sequence.

In some embodiments of the fusion proteins of the disclosure, the antigen sensing region comprises a humanized or fully human antibody. In some embodiments, the antigen sensing region comprises a single chain variable fragment (scFv). In some embodiments, the antigen sensing region comprises an antibody mimetic. In some embodiments, the antigen sensing region comprises a monobody.

In some embodiments of the fusion proteins of the disclosure, the extracellular domain further comprises one or more of a hinge region, a spacer sequence, or a safety switch. In some embodiments, the hinge region comprises a sequence isolated or derived from a CD4 (cluster of differentiation 4) polypeptide, a CD8 (cluster of differentiation 8) polypeptide or a CD28 (cluster of differentiation 28) polypeptide. In some embodiments, the hinge region comprises a sequence isolated or derived from a human sequence. In some embodiments, the hinge region and the spacer sequences are the same domain. In some embodiments, the hinge region and the spacer sequences are not the same domain.

In some embodiments of the fusion proteins of the disclosure, the extracellular domain further comprises one or more of a hinge region, a spacer sequence, or a safety switch. In some embodiments, the spacer sequence comprises a sequence isolated or derived from a CD4 polypeptide, a CD8 polypeptide, a CD28 polypeptide. In some embodiments, the spacer sequence comprises a sequence isolated or derived from a human sequence.

In some embodiments of the fusion proteins of the disclosure, the extracellular domain further comprises one or more of a hinge region, a spacer sequence, or a safety switch. In some embodiments, the safety switch comprises a sequence isolated or derived from an epidermal growth factor receptor (EGFR) polypeptide. In some embodiments, the safety switch comprises a truncated EGFR (EGFRt) polypeptide. In some embodiments, when EGFRt binds rituximab, the combination results in cell death. In some embodiments, the EGFRt comprises the sequence of MLLLVTSLLLCELPHPAFLLIPRKVCNGIGIGE. In some embodiments, the safety switch comprises a sequence isolated or derived from a human sequence.

In some embodiments of the fusion proteins of the disclosure, the transmembrane domain comprises a sequence isolated or derived from a CD4 polypeptide, a CD8 polypeptide, a CD28 polypeptide. In some embodiments, the transmembrane domain comprises a sequence isolated or derived from a human sequence.

In some embodiments of the fusion proteins of the disclosure, the transmembrane domain comprises a sequence isolated or derived from a CD4 polypeptide, a CD8 polypeptide, a CD28 polypeptide. In some embodiments, the intracellular domain comprises one or more costimulatory domain(s). In some embodiments, the one or more costimulatory domain(s) comprises a sequence isolated or derived from a CD3ζ (cluster of differentiation 3 zeta) polypeptide. In some embodiments, the one or more costimulatory domain(s) comprises a sequence isolated or derived from a CD28 polypeptide, a 4-1BB (cluster of differentiation 137) polypeptide, an ICOS (Inducible T Cell Costimulator) polypeptide, an OX40 polypeptide, or a CD27 (cluster of differentiation 27) polypeptide. In some embodiments, the one or more costimulatory domain(s) comprises (a) a first costimulatory domain comprising sequence isolated or derived from a CD28 polypeptide, a 4-1BB polypeptide, or an ICOS polypeptide; and (b) a second costimulatory domain comprising sequence isolated or derived from a 4-1BB polypeptide, an OX40 polypeptide, or a CD27 polypeptide. In some embodiments, the one or more costimulatory domain(s) comprise(s) a sequence isolated or derived from a human sequence.

In some embodiments of the fusion proteins of the disclosure, the chimeric polypeptide comprises an intracellular domain further comprising an inducible cytokine domain. In some embodiments, the inducible cytokine domain comprises a nuclear factor of activated T-cells (NFAT) polypeptide capable of inducing expression of an IL-12 cytokine.

In some embodiments of the fusion proteins of the disclosure, the chimeric polypeptide comprises an intracellular domain further comprising an intracellular domain of a cytokine receptor. In some embodiments, the intracellular domain of a cytokine receptor comprises an IL-2 receptor beta (IL-2Rβ) chain fragment. In some embodiments, the chimeric polypeptide comprises an intracellular domain further comprising a Signal Transducer and Activator of Transcription (STAT3/5) binding motif.

In some embodiments of the fusion proteins of the disclosure, the chimeric polypeptide comprises an intracellular domain further comprising at least one immunoreceptor tyrosine-based activation motif (ITAM) sequence.

In some embodiments of the fusion proteins of the disclosure, the fusion protein comprises, from amino to carboxy termini, the chimeric polypeptide, a linker, and the dimerization domain. In some embodiments, the linker comprises one or more of a DNA sequence, an RNA sequence, an amino acid sequence, and a polymer. In some embodiments, the linker: comprises a sequence of GGGGS; or comprises a length of between 2 and 20 amino acids; or comprises a sequence comprising glycine (G) and serine (S). In some embodiments, the linker comprises an oligomerization domain. In some embodiments, the oligomerization domain comprises the sequence of

SEYEIRKALEELKASTAELKRATASLRASTEELKKNPSEDALVEN
NRLIVEHNAIIVENNRIIAAVLELIVRAIK (trimer);
TRTEIIRELERSLREQEELAKRLKELLRELERLQREGSSDEDVRE
LLREIKELVEEIEKLAREQKYLVEELKRQD (dimer);
TRRKQEMKRLKKEMEKIREETEEVKKEIEESKKRPQSESAKNLIL
IMQLLINQIRLLALQIRMLALQLQE (pentamer);
or
TEDEIRKLRKLLEEAEKKLKKLEDKTRRSEEISKTDDDPKAQSLQ
LIAESLMLIAESLLIIAISLLLSSRNG (hexamer).

In some embodiments of the fusion proteins of the disclosure, including those comprising a chimeric polypeptide, the dimerization domain comprises an NS3a polypeptide. In some embodiments, the NS3a polypeptide comprises a sequence of

(a)
MAKGSVVIVGRINLSGDTAYSQQTRGLLGCIITSATGRDKNQVDG
EVQVLSTATQSFLATCVNGVCWTVYHGAGSKTLAGPKGPITQMYT
NVDQDLVGWPAPPGARSMTPCTCGSSDLYLVTRHADVIPVRRRGD
SRGSLLSPRPVSYLKGSAGGPLLCPSGHVVGIFRAAVCTRGVAKA
VDFIPVESMETTMRSP;
or
(b)
MKKKGSVVIVGRINLSGDTAYSQQTRGLEGCQETSQTGRDKNQVE
GEVQVVSTATQSFLATSINGVLWTVYHGAGTRTIASPKGPVTQMY
TNVDKDLVGWQAPQGSRSLTPCTCGSSDLYLVTRHADVIPVRRRG
DSRGSLLSPRPISYLKGSAGGPLLCPAGHAVGIFRAAVSTRGVAK
AVDFIPVESLETTMRSP;
or
(c)
KKKGSVVIVGRINLSGDTAYAQQTRGEEGCQETSQTGRDKNQVEG
EVQIVSTATQTFLATSINGVLWTVYHGAGTRTIASPKGPVTQMYT
NVDKDLVGWQAPQGSRSLTPCTCGSSDLYLVTRHADVIPVRRRGD
SRGSLLSPRPISYLKGSAGGPLLCPAGHAVGIFRAAVSTRGVAKA
VDFIPVESLETTMRSP.

In some embodiments of the fusion proteins of the disclosure, including those comprising a chimeric polypeptide, the dimerization domain comprises a DNCR polypeptide. In some embodiments, the DNCR polypeptide comprises a sequence of

SSDEEEARELIERAKEAAERAQEAAERTGDPRVRELARELKRLAQ
EAAEEVKRDPSSSDVNEALKLIVEAIEAAVDALEAAERTGDPEVR
ELARELVRLAVEAAEEVQRNPSSSDVNEALHSIVYAIEAAIFALE
AAERTGDPEVRELARELVRLAVEAAEEVQRNPSSRNVEHALMRIV
LAIYLAEENLREAEESGDPEKREKARERVREAVERAEEVQRDPSG
WLNH.

In some embodiments of the fusion proteins of the disclosure, including those comprising a chimeric polypeptide, the dimerization domain comprises a GNCR polypeptide. In some embodiments, the GNCR polypeptide comprises a sequence of

DIEKLCKKAEEEAKEAQEKADELRQRHPDSQAAEDAEDLANLAVA
AVLTACLLAQEHPNADIAKLCIKAASEAAEAASKAAELAQRHPDS
QAARDAIKLASQAARAVILAIMLAAENPNADIAKLCIKAASEAAE
AASKAAELAQRHPDSQAARDAIKLASQAAEAVERAIWLAAENPNA
DIAKKCIKAASEAAEEASKAAEEAQRHPDSQKARDEIKEASQKAE
EVKERCKS.

In some embodiments of the fusion proteins of the disclosure, including those comprising a chimeric polypeptide, the dimerization domain comprises an apo NS3a reader (ANR) polypeptide. In some embodiments, the ANR polypeptide comprises a sequence of

GELDELVYLLDGPGYDPIHSDVVTRGGSHLFNF.

In some embodiments of the fusion proteins of the disclosure, including those comprising a chimeric polypeptide, the fusion protein further comprises a killing domain. In some embodiments, the killing domain comprises a sequence isolated or derived from a Caspase-9 (Cas9) polypeptide, a Caspase-1 (Cas1 polypeptide), a Caspase-4 (Cas4) polypeptide, a Receptor Interacting Serine/Threonine Kinase 1 (RIPK1), a Receptor Interacting Serine/Threonine Kinase 3 (RIPK3) polypeptide or a catalytic domain thereof.

In some embodiments of the fusion proteins of the disclosure, including those comprising a chimeric polypeptide, the fusion protein further comprises a cleavable peptide. In some embodiments, the cleavable peptide comprises a P2A sequence or a T2A sequence. In some embodiments, the P2A sequence comprises the sequence of GSGATNFSLLKQAGDVEENPGP. In some embodiments, the T2A sequence comprises the sequence of GSGEGRGSLLTCGDVEENPG.

The disclosure provides a second fusion protein comprising a signaling domain operably-linked to a dimerization domain, wherein, (a) in the presence of a small molecule inducer, the dimerization domain is capable of forming a dimer with the dimerization domain of a first fusion protein of the disclosure or (b) in the presence of an inhibitor, a dimer comprising the dimerization domain of the second fusion protein and the dimerization domain of the first fusion protein may be disrupted.

In some embodiments of the disclosure, the second fusion protein further comprises one or more of an adaptor sequence, a transmembrane domain, and a costimulatory domain. In some embodiments, the adaptor sequence comprises a sequence isolated or derived from a DAP10 polypeptide. In some embodiments, the DAP10 polypeptide comprises a sequence of MIHLGHILFLLLLPVAAAQTTPGERSSLPAFYPGTSGSCSGCGSLSLPLLAGLVAADAVA SLLIVGAVFLCARPRRSPAQEDGKVYINMPGRG or a sequence having at least 90% identity thereto. In some embodiments, the adaptor sequence comprises a sequence isolated or derived from an epidermal growth factor receptor (EGFR) polypeptide. In some embodiments, the safety switch comprises a truncated EGFR (EGFRt) polypeptide. In some embodiments, when EGFRt binds rituximab, the combination results in cell death. In some embodiments, the EGFRt comprises the sequence of MLLLVTSLLLCELPHPAFLLIPRKVCNGIGIGE. In some embodiments, the adaptor sequence comprises a sequence isolated or derived from a human sequence.

In some embodiments of the disclosure, the second fusion protein further comprises one or more of an adaptor sequence, a transmembrane domain, and a costimulatory domain. In some embodiments, the transmembrane domain comprises a sequence isolated or derived from a CD4 polypeptide, a CD8 polypeptide, a CD28 polypeptide. In some embodiments, the transmembrane domain comprises a sequence isolated or derived from a human sequence.

In some embodiments of the disclosure, the second fusion protein further comprises one or more of an adaptor sequence, a transmembrane domain, and a costimulatory domain. In some embodiments, the costimulatory domain comprises a sequence isolated or derived from a CD3ζ (cluster of differentiation 3 zeta) polypeptide. In some embodiments, the costimulatory domain comprises a sequence isolated or derived from a CD28 polypeptide, a 4-1BB polypeptide, an ICOS polypeptide, an OX40 polypeptide, or a CD27 polypeptide. In some embodiments, the costimulatory domain comprises a sequence isolated or derived from a human sequence.

In some embodiments of the disclosure, the second fusion protein comprises, from amino to carboxy termini, the dimerization domain, a linker and the regulation domain. In some embodiments, the linker comprises one or more of a DNA sequence, an RNA sequence, an amino acid sequence, and a polymer. In some embodiments, the linker: comprises a sequence of GGGGS; or comprises a length of between 2 and 20 amino acids; or comprises a sequence comprising glycine (G) and serine (S). In some embodiments, the linker comprises an oligomerization domain. In some embodiments, the oligomerization domain comprises the sequence of

SEYEIRKALEELKASTAELKRATASLRASTEELKKNPSEDALVEN
NRLIVEHNAIIVENNRIIAAVLELIVRAIK (trimer);
TRTEIIRELERSLREQEELAKRLKELLRELERLQREGSSDEDVRE
LLREIKELVEEIEKLAREQKYLVEELKRQD (dimer);
TRRKQEMKRLKKEMEKIREETEEVKKEIEESKKRPQSESAKNLIL
IMQLLINQIRLLALQIRMLALQLQE (pentamer);
or
TEDEIRKLRKLLEEAEKKLKKLEDKTRRSEEISKTDDDPKAQSLQ
LIAESLMLIAESLLIIAISLLLSSRNG (hexamer).

In some embodiments of the second fusion protein of the disclosure, the dimerization domain comprises an NS3a polypeptide. In some embodiments, the NS3a polypeptide comprises a sequence of

(a)
MAKGSVVIVGRINLSGDTAYSQQTRGLLGCIITSATGRDKNQVDG
EVQVLSTATQSFLATCVNGVCWTVYHGAGSKTLAGPKGPITQMYT
NVDQDLVGWPAPPGARSMTPCTCGSSDLYLVTRHADVIPVRRRGD
SRGSLLSPRPVSYLKGSAGGPLLCPSGHVVGIFRAAVCTRGVAKA
VDFIPVESMETTMRSP;
or
(b)
MKKKGSVVIVGRINLSGDTAYSQQTRGLEGCQETSQTGRDKNQVE
GEVQVVSTATQSFLATSINGVLWTVYHGAGTRTIASPKGPVTQMY
TNVDKDLVGWQAPQGSRSLTPCTCGSSDLYLVTRHADVIPVRRRG
DSRGSLLSPRPISYLKGSAGGPLLCPAGHAVGIFRAAVSTRGVAK
AVDFIPVESLETTMRSP;
or
(c)
KKKGSVVIVGRINLSGDTAYAQQTRGEEGCQETSQTGRDKNQVEG
EVQIVSTATQTFLATSINGVLWTVYHGAGTRTIASPKGPVTQMYT
NVDKDLVGWQAPQGSRSLTPCTCGSSDLYLVTRHADVIPVRRRGD
SRGSLLSPRPISYLKGSAGGPLLCPAGHAVGIFRAAVSTRGVAKA
VDFIPVESLETTMRSP.

In some embodiments, the first fusion protein comprises a dimerization domain comprising a DNCR polypeptide, a GNCR polypeptide or an ANR polypeptide.

In some embodiments of the second fusion protein of the disclosure, the dimerization domain comprises a DNCR polypeptide. In some embodiments, the DNCR polypeptide comprises a sequence of

SSDEEEARELIERAKEAAERAQEAAERTGDPRVRELARELKRLAQ
EAAEEVKRDPSSSDVNEALKLIVEAIEAAVDALEAAERTGDPEVR
ELARELVRLAVEAAEEVQRNPSSSDVNEALHSIVYAIEAAIFALE
AAERTGDPEVRELARELVRLAVEAAEEVQRNPSSRNVEHALMRIV
LAIYLAEENLREAEESGDPEKREKARERVREAVERAEEVQRDPSG
WLNH.

In some embodiments of the second fusion protein of the disclosure, the dimerization domain comprises a GNCR polypeptide. In some embodiments, the GNCR polypeptide comprises a sequence of

DIEKLCKKAEEEAKEAQEKADELRQRHPDSQAAEDAEDLANLAVA
AVLTACLLAQEHPNADIAKLCIKAASEAAEAASKAAELAQRHPDS
QAARDAIKLASQAARAVILAIMLAAENPNADIAKLCIKAASEAAE
AASKAAELAQRHPDSQAARDAIKLASQAAEAVERAIWLAAENPNA
DIAKKCIKAASEAAEEASKAAEEAQRHPDSQKARDEIKEASQKAE
EVKERCKS.

In some embodiments of the second fusion protein of the disclosure, the dimerization domain comprises an apo NS3a reader (ANR) polypeptide. In some embodiments, the ANR polypeptide comprises a sequence of GELDELVYLLDGPGYDPIHSDVVTRGGSHLFNF.

In some embodiments of the second fusion protein of the disclosure, including those wherein the dimerization domain comprises a DNCR polypeptide, a GNCR polypeptide or an ANR polypeptide, the first fusion protein comprises a dimerization domain comprising a NS3a polypeptide.

In some embodiments of the second fusion protein of the disclosure, the second fusion protein further comprises a killing domain. In some embodiments, the killing domain comprises a sequence isolated or derived from a Cas9 polypeptide, a Cas1 polypeptide, a Cas4 polypeptide, a RIPK1 polypeptide, a RIPK3 polypeptide or a catalytic domain thereof.

In some embodiments of the second fusion protein of the disclosure, the second fusion protein further comprises a cleavable peptide. In some embodiments, the cleavable peptide comprises a P2A sequence or a T2A sequence. In some embodiments, the P2A sequence comprises the sequence of GSGATNFSLLKQAGDVEENPGP. In some embodiments, the T2A sequence comprises the sequence of GSGEGRGSLLTCGDVEENPG.

The disclosure provides a nucleic acid encoding the fusion protein of the disclosure. In some embodiments, a nucleic acid encodes a first fusion protein of the disclosure. In some embodiments, a nucleic acid encodes a second fusion protein of the disclosure. In some embodiments, a nucleic acid encodes a first fusion protein and a second fusion protein of the disclosure.

In some embodiments of the nucleic acids of the disclosure, the nucleic acid further comprises an Internal Ribosome Entry Sequence (IRES). In some embodiments, the IRES comprises the sequence of

CCTCTCCCTCCCCCCCCCCTAACGTTACTGGCCGAAGCCGCTTGG
AATAAGGCCGGTGTGCGTTTGTCTATATGTGATTTTCCACCATAT
TGCCGTCTTTTGGCAATGTGAGGGCCCGGAAACCTGGCCCTGTCT
TCTTGACGAGCATTCCTAGGGGTCTTTCCCCTCTCGCCAAAGGAA
TGCAAGGTCTGTTGAATGTCGTGAAGGAAGCAGTTCCTCTGGAAG
CTTCTTGAAGACAAACAACGTCTGTAGCGACCCTTTGCAGGCAGC
GGAACCCCCCACCTGGCGACAGGTGCCTCTGCGGCCAAAAGCCAC
GTGTATAAGATACACCTGCAAAGGCGGCACAACCCCAGTGCCACG
TTGTGAGTTGGATAGTTGTGGAAAGAGTCAAATGGCTCTCCTCAA
GCGTATTCAACAAGGGGCTGAAGGATGCCCAGAAGGTACCCCATT
GTATGGGATCTGATCTGGGGCCTCGGTGCACATGCTTTACATGTG
TTTAGTCGAGGTTAAAAAAACGTCTAGGCCCCCCGAACCACGGGG
ACGTGGTTTTCCTTTGAAAAACACGATGATAAGCTTGCCACAACC
C.

in some embodiments of the nucleic acids of the disclosure, the nucleic acid further comprises one or more of a promoter, an enhancer, an intron, an exon, an untranslated region (UTR), and a post translational regulatory element (PRE). In some embodiments, the promoter comprises an inducible promoter. In some embodiments, the inducible promoter comprises a sequence isolated or derived from a YB_TATA promoter, human beta globin promoter (huBG), minIL2 promoter, minimal CMV (minCMV) promoter, and TRE3G promoter. In some embodiments, the promoter comprises a constitutive promoter. In some embodiments, the constitutive promoter comprises a sequence isolated or derived from a MND promoter, a hPGK promoter, a CMV promoter, a CAG promoter, a SFFV promoter, an EF1alpha promoter, a UBC promoter, and a CD43 promoter.

The disclosure provides a vector, comprising a nucleic acid of the disclosure. In some embodiments, the nucleic acid encodes a fusion protein of the disclosure. In some embodiments, the nucleic acid encodes a first fusion protein and a second fusion protein of the disclosure. In some embodiments, the vector comprises a first nucleic acid encoding a first fusion protein of the disclosure and a second nucleic acid encoding a second nucleic acid of the disclosure. In some embodiments, a first vector comprises a nucleic acid encoding a first fusion protein of the disclosure and a second vector comprises a second fusion protein of the disclosure.

In some embodiments of the vectors of the disclosure, the vector comprises an expression vector capable of driving expression of the nucleic acid in a mammalian cell. In some embodiments, the expression vector comprises a plasmid. In some embodiments, the expression vector is a cloning vector.

In some embodiments of the vectors of the disclosure, the vector comprises a delivery vector capable of introducing the nucleic acid to a mammalian cell. In some embodiments, the delivery vector comprises one or more of a viral vector, a non-viral vector, a liposome, a micelle, a polymersome, and a nanoparticle. In some embodiments, the viral vector comprises one or more sequences isolated or derived from a viral genome.

The disclosure provides a cell comprising a fusion protein of the disclosure, a nucleic acid of the disclosure or the vector of the disclosure. In some embodiments, the cell is a mammalian cell. In some embodiments, the cell is a human cell. In some embodiments, the cell is a somatic cell. In some embodiments, the cell is a stem cell. In some embodiments, the cell is not a human embryonic stem cell. In some embodiments, the cell is ex vivo or in vitro. In some embodiments, the cell is in vivo. In some embodiments, the cell is an immune cell. In some embodiments, the cell is a T-cell or a precursor thereof. In some embodiments, the precursor is a hematopoietic stem cell (HSC).

In some embodiments of cells of the disclosure, including those cells expressing a first fusion protein of the disclosure and a second fusion protein of the disclosure, the cell further comprises a small molecule inducer. In some embodiments, the small molecule inducer comprises danoprevir. In some embodiments, including those wherein the small molecule inducer comprises danoprevir, the dimerization domain of either the first fusion protein or the second fusion protein comprises a DNCR polypeptide. In some embodiments, the small molecule inducer comprises grazoprevir. In some embodiments, including those wherein the small molecule inducer comprises grazoprevir, the dimerization domain of either the first fusion protein or the second fusion protein comprises a GNCR polypeptide.

In some embodiments of cells of the disclosure, including those cells expressing a first fusion protein of the disclosure and a second fusion protein of the disclosure, the cell further comprises an inhibitor. In some embodiments, the inhibitor comprises an NS3a inhibitor. In some embodiments, the inhibitor comprises a small molecule. In some embodiments, (a) the first fusion protein comprises a dimerization domain comprising an NS3a polypeptide and the second fusion protein comprises a dimerization domain comprising an ANR polypeptide; or (b) the first fusion protein comprises a dimerization domain comprising an ANR polypeptide and the second fusion protein comprises a dimerization domain comprising an NS3a polypeptide; and the inhibitor comprises danoprevir or grazoprevir.

The disclosure provides a composition comprising a fusion protein of the disclosure, a nucleic acid of the disclosure, a vector of the disclosure, or a cell of the disclosure.

In some embodiments of compositions of the disclosure, including those compositions comprising a first fusion protein of the disclosure and a second fusion protein of the disclosure, the composition further comprises a small molecule inducer. In some embodiments, the small molecule inducer comprises danoprevir. In some embodiments, including those wherein the small molecule inducer comprises danoprevir, the dimerization domain of either the first fusion protein or the second fusion protein comprises a DNCR polypeptide. In some embodiments, the small molecule inducer comprises grazoprevir. In some embodiments, including those wherein the small molecule inducer comprises grazoprevir, the dimerization domain of either the first fusion protein or the second fusion protein comprises a GNCR polypeptide.

In some embodiments of compositions of the disclosure, including those compositions expressing a first fusion protein of the disclosure and a second fusion protein of the disclosure, the composition further comprises an inhibitor. In some embodiments, the inhibitor comprises an NS3a inhibitor. In some embodiments, the inhibitor comprises a small molecule. In some embodiments, (a) the first fusion protein comprises a dimerization domain comprising an NS3a polypeptide and the second fusion protein comprises a dimerization domain comprising an ANR polypeptide; or (b) the first fusion protein comprises a dimerization domain comprising an ANR polypeptide and the second fusion protein comprises a dimerization domain comprising an NS3a polypeptide; and the inhibitor comprises danoprevir or grazoprevir.

The disclosure provides a pharmaceutical composition comprising a composition of the disclosure and a pharmaceutically-acceptable carrier.

The disclosure provides a use of a fusion protein of the disclosure, a nucleic acid of the disclosure, a vector of the disclosure, a cell of the disclosure, a composition of the disclosure, or the pharmaceutical composition of the disclosure in the manufacture of a medicament for the treatment of a disease or disorder. In some embodiments, the disease or disorder comprises one or more of an autoimmune disease or disorder; an inflammatory disease or disorder; an immunodeficiency disease or disorder; and a proliferative disease or disorder. In some embodiments, the disease or disorder comprises a cancer. In some embodiments, the disease or disorder comprises an infection or a disease or disorder caused by the infectious disease. In some embodiments, the disease or disorder comprises a genetic disease or disorder.

The disclosure provides a use of a fusion protein of the disclosure, a nucleic acid of the disclosure, a vector of the disclosure, a cell of the disclosure, a composition of the disclosure, or the pharmaceutical composition of the disclosure for the treatment of a disease or disorder.

In some embodiments of the uses of the disclosure, the disease or disorder comprises one or more of an autoimmune disease or disorder; an inflammatory disease or disorder; an immunodeficiency disease or disorder; an ischemic disease or disorder; a blood disease or disorder; a bone disease or disorder; a neurological disease or disorder; a cardiac disease or disorder; a vascular disease or disorder; a metabolic disease or disorder; a dermatological disease or disorder; a digestive disease or disorder; a mitochondrial disease or disorder; a muscle disease or disorder; a liver disease or disorder; a kidney disease or disorder; a hearing disease or disorder; an ophthalmic disease or disorder; and a proliferative disease or disorder.

In some embodiments of the uses of the disclosure, the disease or disorder comprises a cancer. In some embodiments, the cancer comprises one or more of Acute Lymphocytic Leukemia (ALL) in Adults, Acute Myeloid Leukemia (AML) in Adults, Adrenal Cancer, Anal Cancer, Basal and Squamous Cell Skin Cancer, Bile Duct Cancer, Bladder Cancer, Bone Cancer, Brain and Spinal Cord Tumors in Adults, Brain and Spinal Cord Tumors in Children, Breast Cancer, Breast Cancer in Men, Cancer in Adolescents. Cancer in Children, Cancer in Young Adults, Cancer of Unknown Primary, Cervical Cancer, Chronic Lymphocytic Leukemia (CLL), Chronic Myeloid Leukemia (CML), Chronic Myelomonocytic Leukemia (CMML), Colorectal Cancer, Endometrial Cancer, Esophagus Cancer, Ewing Family of Tumors, Eye Cancer (Ocular Melanoma), Gallbladder Cancer, Gastrointestinal Neuroendocrine (Carcinoid) Tumors, Gastrointestinal Stromal Tumor (GIST), Head and Neck Cancers, Hodgkin Lymphoma, Kaposi Sarcoma, Kidney Cancer, Laryngeal and Hypopharyngeal Cancer, Leukemia, Leukemia in Children, Liver Cancer, Lung Cancer, Lung Carcinoid Tumor, Lymphoma, Lymphoma of the Skin, Malignant Mesothelioma, Melanoma Skin Cancer, Merkel Cell Skin Cancer, Multiple Myeloma, Myelodysplastic Syndromes, Nasal Cavity and Paranasal Sinuses Cancer, Nasopharyngeal Cancer, Neuroblastoma, Non-Hodgkin Lymphoma, Non-Hodgkin Lymphoma in Children, Oral Cavity (Mouth) and Oropharyngeal (Throat) Cancer, Osteosarcoma, Ovarian Cancer, Pancreatic Cancer, Pancreatic Neuroendocrine Tumor (NET), Penile Cancer, Pituitary Tumors, Prostate Cancer, Retinoblastoma, Rhabdomyosarcoma, Salivary Gland Cancer, Skin Cancer, Small Intestine Cancer, Soft Tissue Sarcoma, Stomach Cancer, Testicular Cancer, Thymus Cancer, Thyroid Cancer, Uterine Sarcoma, Vaginal Cancer, Vulvar Cancer, Waldenstrom Macroglobulinemia and Wilms Tumor.

In some embodiments of the uses of the disclosure, the disease or disorder comprises an infection or a disease or disorder caused by the infectious disease.

In some embodiments of the uses of the disclosure, the disease or disorder comprises a genetic disease or disorder.

The disclosure provides a method of treating a disease or a disorder, comprising administering to a subject an effective amount of a fusion protein of the disclosure, a nucleic acid of the disclosure, a vector of the disclosure, a cell of the disclosure, a composition of the disclosure, or the pharmaceutical composition of the disclosure, wherein a severity of a sign or symptom of the disease or disorder is decreased, thereby treating the disease or disorder.

The disclosure provides a method of preventing a disease or a disorder, comprising administering to a subject an effective amount of a fusion protein of the disclosure, a nucleic acid of the disclosure, a vector of the disclosure, a cell of the disclosure, a composition of the disclosure, or the pharmaceutical composition of the disclosure, or the pharmaceutical composition of the disclosure, wherein an onset or a relapse of a sign or symptom of the disease or disorder is delayed or inhibited, thereby preventing the disease or disorder.

In some embodiments of the methods of the disclosure, the disease or disorder comprises one or more of an autoimmune disease or disorder; an inflammatory disease or disorder; an immunodeficiency disease or disorder; an ischemic disease or disorder; a blood disease or disorder; a bone disease or disorder; a neurological disease or disorder; a cardiac disease or disorder; a vascular disease or disorder; a metabolic disease or disorder; a dermatological disease or disorder; a digestive disease or disorder; a mitochondrial disease or disorder; a muscle disease or disorder; a liver disease or disorder; a kidney disease or disorder; a hearing disease or disorder; an ophthalmic disease or disorder; and a proliferative disease or disorder.

In some embodiments of the methods of the disclosure, the disease or disorder comprises a cancer. In some embodiments, the cancer comprises one or more of Acute Lymphocytic Leukemia (ALL) in Adults, Acute Myeloid Leukemia (AML) in Adults, Adrenal Cancer, Anal Cancer, Basal and Squamous Cell Skin Cancer, Bile Duct Cancer, Bladder Cancer, Bone Cancer, Brain and Spinal Cord Tumors in Adults, Brain and Spinal Cord Tumors in Children, Breast Cancer, Breast Cancer in Men, Cancer in Adolescents. Cancer in Children, Cancer in Young Adults, Cancer of Unknown Primary, Cervical Cancer, Chronic Lymphocytic Leukemia (CLL), Chronic Myeloid Leukemia (CML), Chronic Myelomonocytic Leukemia (CMML), Colorectal Cancer, Endometrial Cancer, Esophagus Cancer, Ewing Family of Tumors, Eye Cancer (Ocular Melanoma), Gallbladder Cancer, Gastrointestinal Neuroendocrine (Carcinoid) Tumors, Gastrointestinal Stromal Tumor (GIST), Head and Neck Cancers, Hodgkin Lymphoma, Kaposi Sarcoma, Kidney Cancer, Laryngeal and Hypopharyngeal Cancer, Leukemia, Leukemia in Children, Liver Cancer, Lung Cancer, Lung Carcinoid Tumor, Lymphoma, Lymphoma of the Skin, Malignant Mesothelioma, Melanoma Skin Cancer, Merkel Cell Skin Cancer, Multiple Myeloma, Myelodysplastic Syndromes, Nasal Cavity and Paranasal Sinuses Cancer, Nasopharyngeal Cancer, Neuroblastoma, Non-Hodgkin Lymphoma, Non-Hodgkin Lymphoma in Children, Oral Cavity (Mouth) and Oropharyngeal (Throat) Cancer, Osteosarcoma, Ovarian Cancer, Pancreatic Cancer, Pancreatic Neuroendocrine Tumor (NET), Penile Cancer, Pituitary Tumors, Prostate Cancer, Retinoblastoma, Rhabdomyosarcoma, Salivary Gland Cancer, Skin Cancer, Small Intestine Cancer, Soft Tissue Sarcoma, Stomach Cancer, Testicular Cancer, Thymus Cancer, Thyroid Cancer, Uterine Sarcoma, Vaginal Cancer, Vulvar Cancer, Waldenstrom Macroglobulinemia and Wilms Tumor.

In some embodiments of the methods of the disclosure, the disease or disorder comprises an infection or a disease or disorder caused by the infectious disease.

In some embodiments of the methods of the disclosure, the disease or disorder comprises a genetic disease or disorder.

The disclosure provides a method of preventing a disease or a disorder, comprising administering to a subject an effective amount of a fusion protein of the disclosure, a nucleic acid of the disclosure, a vector of the disclosure, a cell of the disclosure, a composition of the disclosure, or the pharmaceutical composition of the disclosure, or the pharmaceutical composition of the disclosure, wherein an onset or a relapse of a sign or symptom of the disease or disorder is delayed or inhibited, thereby preventing the disease or disorder.

The disclosure provides a polynucleotide set comprising one or more vectors comprising: (a) a first polynucleotide encoding a first fusion protein comprising a first signaling domain linked to a first dimerization polypeptide; and (b) a second polynucleotide encoding a second fusion protein comprising a second signaling domain or an oligomerization domain linked to a second dimerization polypeptide; and wherein the first and second dimerization polypeptides are selected so that interaction of the first and second dimerization polypeptides is mediated by the presence of a small molecule.

In some embodiments of the polynucleotide sets of the disclosure, a signaling function in a cell is inducible relative to administration of the small molecule to the cell.

In some embodiments of the polynucleotide sets of the disclosure, the small molecule mediates binding of the first and second dimerization polypeptides.

In some embodiments of the polynucleotide sets of the disclosure, the small molecule disrupts binding of the first and second dimerization polypeptides.

In some embodiments of the polynucleotide sets of the disclosure, the small molecule is selected from the group consisting of: danoprevir and grazoprevir.

In some embodiments of the polynucleotide sets of the disclosure, (a) a first small molecule mediates binding of the first and second dimerization polypeptides; (b) a second small molecule disrupts binding of the first and second dimerization polypeptides; (c) the second small molecule can mediate binding of a third dimerization polypeptide to the first dimerization polypeptide, and (d) the third dimerization polypeptide is linked to a signaling domain.

In some embodiments of the polynucleotide sets of the disclosure, the second small molecule disrupts binding of the first and second dimerization polypeptides by out-competing the first small molecule.

In some embodiments of the polynucleotide sets of the disclosure, (a) the first small molecule is grazoprevir or an analog or derivative thereof; and (b) the second small molecule is danoprevir or an analog or derivative thereof.

In some embodiments of the polynucleotide sets of the disclosure, one or both of the first and second fusion proteins is a cytoplasmic protein.

In some embodiments of the polynucleotide sets of the disclosure, one or both of the first and second fusion proteins is a transmembrane protein.

In some embodiments of the polynucleotide sets of the disclosure, one or both of the first and second fusion proteins comprises a transmembrane domain.

In some embodiments of the polynucleotide sets of the disclosure, (a) one of the first and second fusion proteins is a transmembrane protein; and (b) one of the first and second fusion proteins is a cytoplasmic protein.

In some embodiments of the polynucleotide sets of the disclosure, the polynucleotide set comprises multiple signaling domains linked to the first or second dimerization polypeptide.

In some embodiments of the polynucleotide sets of the disclosure, the polynucleotide set comprises a transmembrane receptor with one or multiple protein domains linked to the first or second dimerization polypeptide.

In some embodiments of the polynucleotide sets of the disclosure, the signaling domain linked to the first dimerization polypeptide is identical or substantially identical to the signaling domain linked to the second dimerization polypeptide.

In some embodiments of the polynucleotide sets of the disclosure, the signaling domain linked to the first dimerization polypeptide is different or substantially different from the signaling domain linked to the second dimerization polypeptide.

In some embodiments of the polynucleotide sets of the disclosure, the polynucleotide set comprises at least two vectors comprising: (a) a first vector comprising the polynucleotide encoding the first fusion protein; and (b) a second vector comprising polynucleotide encoding the second fusion protein.

In some embodiments of the polynucleotide sets of the disclosure, a vector backbone comprises the polynucleotide set. In some embodiments, the vector backbone is selected from the group consisting of backbones of adenoviral vectors, lentiviral vectors, baculoviral vectors, Epstein Barr viral vectors, papovaviral vectors, vaccinia viral vectors, herpes simplex viral vectors, retroviral vectors, adeno associated virus (AAV) vectors, and transposon vectors. In some embodiments, the vector backbone comprises a homology directed repair vector.

In some embodiments of the polynucleotide sets of the disclosure, a chromosome comprises the polynucleotide set.

In some embodiments of the polynucleotide sets of the disclosure, the polynucleotide encoding a first fusion protein and the polynucleotide encoding the second fusion protein are separated by a separation element comprising a polynucleotide sequence that prevents fusion of the first fusion protein and the second fusion protein. In some embodiments, the separation element comprises a polynucleotide sequence comprising a ribosomal skipping sequence. In some embodiments, the separation element comprises a polynucleotide sequence comprising at least two ribosomal skipping sequences. In some embodiments, the separation element comprises a polynucleotide sequence comprising P2a and/or T2a. In some embodiments, the separation element comprises a polynucleotide sequence selected from the group consisting of: P2a, T2a, T2a-RFP-P2a, P2a-T2a, T2a-P2a, and IRES. In some embodiments, the separation element comprises a polynucleotide sequence comprising a constitutive promoter.

In some embodiments of the polynucleotide sets of the disclosure, (a) the signaling domains linked to the dimerization polypeptides are part of a chimeric antigen receptor (CAR); and (b) administration of the small molecule modulates T cell signaling function.

In some embodiments of the polynucleotide sets of the disclosure, (a) the first fusion protein comprises NS3a linked to a cytoplasmic end of a transmembrane CAR lacking a CD3ζ domain, (b) the second fusion protein comprises ANR peptide linked to a CD3ζ domain; and wherein the small molecule down-regulates CAR T cell signaling.

In some embodiments of the polynucleotide sets of the disclosure, (a) the first fusion protein comprises NS3a linked to a cytoplasmic end of a transmembrane CAR lacking a CD3ζ domain; (b) the second fusion protein comprises DNCR2 (or designed variants thereof, including but not limited to DNCR2_1 through DNCR2_34, and DNCR2-3rep) or GNCR1 (or designed variants thereof, including but not limited to GNCR1-3rep, G33, and G38) linked to a CD3ζ domain, and wherein administration of the small molecule activates CAR T cell signaling. In some embodiments, the fusion protein further comprises a transmembrane domain and/or a costimulatory domain.

In some embodiments of the polynucleotide sets of the disclosure, (a) first and second signaling domains comprise killing domains; and (b) administration of the small molecule induces cell death.

In some embodiments of the polynucleotide sets of the disclosure, either or both of the first and second signaling domains are selected from the group consisting of: Caspase-9, Caspase-1, Caspase-4, RIPK1, or RIPK3, mutated variants thereof, and combinations of any of the foregoing. In some embodiments, either or both of the first and second signaling domains comprises a catalytic domain of caspase-9 or a variant thereof. In some embodiments, (a) the first signaling domain comprises a catalytic domain of caspase-9 or a variant thereof, and (b) the second signaling domain comprises a catalytic domain of caspase-9 or a variant thereof. In some embodiments, (a) the first signaling domain comprises a catalytic domain of caspase-9 or a variant thereof, and (b) the second dimerization polypeptide is linked to a homo-oligomerization domain. In some embodiments, (a) the first dimerization polypeptide is selected from the group of: DNCR2 (or designed variants thereof, including but not limited to DNCR2_1 through DNCR2_34, and DNCR2-3rep) or GNCR1 (or variants thereof, including but not limited to GNCR1-3rep, G33, and G38) linked to a killing domain, and (b) the second dimerization polypeptide is NS3a and is linked to a homo-oligomerization domain selected from the group of: dimer-NS3aH1, trimer-NS3aH1, pentamer-NS3aH1, hexamer-NS3a, or variants thereof. In some embodiments, (a) the first dimerization polypeptide is selected from the group consisting of: DNCR2 (or designed variants thereof, including but not limited to DNCR2_1 through DNCR2_34, and DNCR2-3rep) or GNCR1 (or variants thereof, including but not limited to GNCR1-3rep, G33, and G38); and (b) the second dimerization polypeptide is NS3a linked to a CAR or other homo-oligomeric receptor.

The disclosure provides a cell comprising the polynucleotide set of the disclosure. In some embodiments, the cell is a prokaryotic cell. In some embodiments, the cell is a yeast cell. In some embodiments, the cell is a mammalian cell. In some embodiments, the cell is a human cell. In some embodiments, the cell is a human cell in vivo. In some embodiments, the cell is a human cell ex vivo. In some embodiments, the cell is a stem cell. In some embodiments, the cell is a pluripotent stem cell. In some embodiments, the cell is an induced pluripotent stem cell. In some embodiments, the cell is a hematopoietic stem cell.

The disclosure provides a method of effecting stem cell differentiation comprising modifying a stem cell using a polypeptide set of the polynucleotide set of the disclosure. In some embodiments, the cell is a cancer cell. In some embodiments, the cell is a non-cancer cell from a human subject diagnosed with cancer. In some embodiments, the cell is an immune cell. In some embodiments, the cell is is selected from the group consisting of: leukocyte, lymphocyte, T cell, regulatory T cell, effector T cell, CD4+ effector T cell, CD8+ effector T cell, memory T cell, autoreactive T cell, exhausted T cell, natural killer T cell, B cell, dendritic cell, and macrophage. In some embodiments, the cell is selected from the group consisting of: cardiac cell, lung cell, muscle cell, epithelial cell, pancreatic cell, skin cell, CNS cell, neuron, myocyte, skeletal muscle cell, smooth muscle cell, liver cell, kidney cell and glial cell.

The disclosure provides a cell genetically modified to express a CAR, comprising a polynucleotide of the disclosure. In some embodiments, the cell is a T cell, a natural killer (NK) cell, a natural killer T (NKT) cell, or an ILC cell.

The disclosure provides a producer cell line wherein cells of the cell line comprise a polynucleotide set of the disclosure.

The disclosure provides a method of producing a polypeptide product of interest from a gene of interest, the method comprising: (a) modifying a cell line using the polynucleotide set of any one of claims 1 to 38 to yield a producer cell line; and (b) culturing the producer cell line to produce the product of interest. In some embodiments, the polypeptide product of interest comprises a therapeutic protein or peptide. In some embodiments, the polypeptide product of interest comprises a virus. In some embodiments, the virus is selected from the group consisting of adenovirus, lentivirus, baculovirus, Epstein Barr virus, papovavirus, vaccinia virus, herpes virus, herpes simplex virus, retrovirus, and adeno-associated virus (AAV).

The disclosure provides a producer cell line wherein cells of the cell line produce a polynucleotide set of the disclosure.

The disclosure provides a producer cell line wherein cells of the cell line produce a polynucleotide set of the disclosure packaged in a viral capsid. In some embodiments, the viral capsid is selected from the group consisting of capsids of an adenovirus, lentivirus, baculovirus, Epstein Barr virus, papovavirus, vaccinia virus, herpes virus, herpes simplex virus, retrovirus, and adeno-associated virus (AAV).

The disclosure provides a viral capsid comprising the polynucleotide set of a polynucleotide set of the disclosure. In some embodiments, the viral capsid is selected from the group consisting of capsids of an adenovirus, lentivirus, baculovirus, Epstein Barr virus, papovavirus, vaccinia virus, herpes virus, herpes simplex virus, retrovirus, and adeno-associated virus (AAV).

The disclosure provides a cell producing a viral capsid of the disclosure. In some embodiments, the viral capsid is selected from the group consisting of capsids of an adenovirus, lentivirus, baculovirus, Epstein Barr virus, papovavirus, vaccinia virus, herpes virus, herpes simplex virus, retrovirus, and adeno-associated virus (AAV).

The disclosure provides a composition comprising a polynucleotide set of the disclosure.

The disclosure provides the use of a composition of the disclosure for treating a subject in need of a CAR therapy.

The disclosure provides a kit comprising the polynucleotide set of the disclosure.

The disclosure provides a method of making an engineered cell comprising transfecting into a cell the polynucleotide of any the polynucleotide set of the disclosure. In some embodiments, the chimeric polypeptide is expressed in the cell. In some embodiments, the method further comprises administering the cell in a subject in need thereof. In some embodiments, the method further comprises administering the small molecule to the subject.

The disclosure provides a method of controlling a T cell-mediated immune response in a subject in need thereof comprising administering to the subject a cell of the disclosure of an effective amount of cells of the disclosure. In some embodiments, the cell is a T cell. In some embodiments, the cell is an autologous T cell. In some embodiments, the method further comprises administering to the subject a small molecule of the disclosure.

The disclosure provides a method of stimulating a T cell-mediated immune response to a target cell population or tissue in a subject, comprising administering to the subject a cell of the disclosure of an effective amount of cells of the disclosure. In some embodiments, the cell is a T cell. In some embodiments, the cell is an autologous T cell. In some embodiments, the method further comprises administering to the subject a small molecule of the disclosure.

The disclosure provides a method of providing an anti-tumor immunity in a subject in need thereof, the method comprising administering to the subject a cell of the disclosure of an effective amount of cells of the disclosure. In some embodiments, the cell is a T cell. In some embodiments, the cell is an autologous T cell. In some embodiments, the method further comprises administering to the subject a small molecule of the disclosure.

The disclosure provides a method of treating cancer in a subject in need thereof comprising administering to the subject a cell of the disclosure of an effective amount of cells of the disclosure. In some embodiments, the cell is a T cell. In some embodiments, the cell is an autologous T cell. In some embodiments, the method further comprises administering to the subject a small molecule of the disclosure.

### BRIEF DESCRIPTION OF DRAWINGS

FIG. 1A is a schematic diagram of a generalized architecture of a split chimeric antigen receptor (CAR) that can be regulated by small molecule drugs to disrupt or induce a cellular signaling activity.

FIG. 1B is a schematic diagram of an off-switch ANR CAR, an on-switch DNCR CAR, and an on-switch GNCR CAR.

FIG. 2 is a schematic diagram of designs to induce homo-oligomerization of killing domains (e.g., caspase-9) in response to small molecule drugs.

FIG. 3 is a plot showing small molecule drug-regulated induction of CD69 expression in CAR expressing Jurkat cells co-cultured with Jeko1 cells or Jeko1-ROR1-KO cells.

FIG. 4A is a panel of plots showing target cell killing by the regulatable CARs in the presence or absence of their cognate inducer or disruptor small molecule drug.

FIG. 4B is a panel of plots showing production of the cytokines IFNγ and IL-2 from the T cells expressing Bcl-xL/Bad, NS3a/ANR, NS3a/GNCR1, or NS3a/DNCR2, respectively, of FIG. 4A.

FIG. 5A is a schematic diagram of a dual-function on-switch/safety-switch regulatable T cell CAR.

FIG. 5B is a pair of plots showing CAR signaling and percent killing, respectively, in the Jurkat CD3ζ-signaling reporter line in the presence and absence of grazoprevir or danoprevir.

FIG. 6A is a pair of plots showing CAR signaling and drug-induced cell death, respectively, in the Jurkat CD3ζ-signaling reporter line in the presence and absence of grazoprevir or danoprevir.

FIG. 6B is a pair of plots showing CAR signaling and drug-induced cell death, respectively, in Jurkat CD3ζ-signaling reporter cells switched from grazoprevir treatment to danoprevir treatment.

FIG. 7A is a schematic diagram of a two-chain direct dimerization design NCDC with caspase-9 killing domains.

FIG. 7B is a plot showing percent killing as a function of drug concentration for the two-chain direct dimerization design NCDC.

FIG. 8A is a schematic diagram of CAR-NS3a-casp9 and homo-oligomeric helical bundle-NS3a caspase-9 kill switches.

FIG. 8B is a plot showing percent killing activity of the templated dimerization designs of FIG. 8A.

FIG. 9A is a schematic diagram of models for minimized DNCR2 versions D1 and D9.

FIG. 9B is a plot showing percent killing activity of the miniaturized designs of FIG. 9A.

FIG. 10 is a pair of plots showing percent killing in Jurkat and primary T cells, respectively, expressing the FKBP(F36V)-fused killing domains at a range of transduction levels.

### DETAILED DESCRIPTION

### Nucleic Acids and Related Terminology

In some embodiments, the terms “Nucleic acid,” “nucleic acid molecule,” “nucleotide,” “nucleotide sequence,” “polynucleotide,” and grammatical variants thereof may be used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible.

In some embodiments, “Nucleic acid,” and in particular a DNA or RNA molecule, may refer only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences are provided according to the normal convention of writing the sequence left to right in the 5′ to 3′ direction along the non-transcribed strand of DNA (i.e., the strand having a sequence homologous to the messenger RNA or mRNA). Unless otherwise indicated, all nucleic acid and nucleotide sequences are written left to right in 5′ to 3′ orientation.

Nucleotides are referred to by their commonly known one-letter symbols recommended by the IUPAC-IUB Biochemical Nomenclature Commission. Accordingly, ‘A’ represents adenine, ‘C’ represents cytosine, ‘G’ represents guanine, ‘T’ represents thymine, and ‘U’ represents uracil.

In some embodiments, the term “Polynucleotide” refers to polymers of nucleotides of any length or type, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”) and ribonucleic acid (“RNA”). It also includes modified, for example by alkylation and/or by capping, and unmodified forms of the polynucleotide. More particularly, “polynucleotide” includes polydeoxyribonucleotides (containing 2-deoxy-D-ribose) and polyribonucleotides (containing D-ribose), including mRNA, whether spliced or unspliced, any other type of polynucleotide which is an N- or C-glycoside of a purine or pyrimidine base, and other polymers containing nucleotide backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some embodiments, a vector of the disclosure comprises a nucleic acid sequence of the disclosure and backbone sequence(s).

In some embodiments, a polynucleotide includes a DNA, e.g., a DNA inserted in a vector. In other aspects, a polynucleotide includes an mRNA. In some aspects, the mRNA is a synthetic mRNA. In some embodiments, the synthetic mRNA includes at least one unnatural nucleobase. In some embodiments, all nucleobases of a certain class have been replaced with unnatural nucleobases (e.g., all uridines in a polynucleotide can be replaced with an unnatural nucleobase, e.g., 5-methoxy uridine).

In some embodiments, the term “Expression” refers to the transcription and/or translation of a particular nucleotide sequence driven by a promoter.

In some embodiments, the term “Expression vector” refers to a plasmid, virus, or other nucleic acid designed for polypeptide expression in a cell. The vector or construct is used to introduce a gene into a host cell whereby the vector will interact with polymerases in the cell to express the protein encoded in the vector/construct. The expression vector may exist in the cell extrachromosomally or may be integrated into the chromosome. Expression vectors may include additional sequences which render the vector suitable for replication and integration in prokaryotes, eukaryotes, or preferably both (e.g., shuttle vectors). The polynucleotides of the disclosure may be provided as components of expression vectors.

In some embodiments, the term “Cloning vector” refers to a plasmid, virus, or other nucleic acid designed for producing copies of a polynucleotide. Cloning vectors may contain transcription and translation initiation sequences, transcription and translation termination sequences and a polyadenylation signal. Such constructs will typically include a 5′ LTR, a tRNA binding site, a packaging signal, an origin of second-strand DNA synthesis, and a 3′ LTR or a portion thereof. The polynucleotides of the disclosure may be provided as components of cloning vectors, which may be used to produce the polynucleotides of the disclosure.

In some embodiments, “Promoter” refers to a nucleotide sequence which indicates where transcription of a gene is initiated and in which direction transcription will continue.

In some embodiments, “Encoding” refers to an ability of specific sequences of nucleotides in a polynucleotide (e.g. a gene, cDNA, or mRNA) to serve as templates for synthesis of other polymers and macromolecules in biological processes having either a defined sequence of nucleotides (e.g., rRNA, tRNA and mRNA) or a defined sequence of amino acids. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a nucleotide sequence “encoding an amino acid sequence,” (including a polynucleotide “encoding” a chimeric polypeptide of the disclosure), includes all nucleotide sequences that are degenerate versions of each other and that encode the same amino acid sequence.

### Polypeptides and Related Terminology

Amino acids are referred to by either their commonly known three letter symbols or by the one-letter symbols recommended by the IUPAC-IUB Biochemical Nomenclature Commission. The amino acid residues are abbreviated as follows, where the abbreviations are shown in parentheses: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Amino acid sequences are written left to right in amino to carboxy orientation.

In some embodiments, the term “Polypeptide” refers to a sequence of amino acid subunits. In some embodiments, a “peptide” may comprise at most 50 amino acids. In some embodiments, a “peptide” may comprise about 5, 10, 15, 20, 25, 30, 35, 40, 45, or 50 amino acids. The term “Polypeptide,” may refer to proteins, polypeptides, and peptides of any length, size, structure, or function. The terms “Polypeptide,” “peptide,” and “protein” may be used interchangeably.

In some embodiments, polypeptides of the disclosure comprise naturally or synthetically created or modified amino acids, for example, disulfide bond formation, glycosylation, lipidation, acetylation, phosphorylation, or any other manipulation or modification, such as conjugation with a labeling component. In some embodiments, polypeptides of the disclosure comprise one or more amino acid residues are artificial chemical analogs of a corresponding naturally occurring amino acid (including, for example, synthetic amino acids such as homocysteine, ornithine, p-acetylphenylalanine, D-amino acids, and creatine), as well as other modifications known in the art. In some embodiments, polypeptides of the disclosure comprise gene products, homologs, orthologs, paralogs, fragments and other equivalents, variants, and analogs of the foregoing. In some embodiments, polypeptides of the disclosure comprise a single polypeptide or can be a multi-molecular complex such as a dimer, trimer or tetramer. In some embodiments, polypeptides of the disclosure comprise single-chain or multi-chain polypeptides. In some embodiments, polypeptides of the disclosure comprise disulfide linkages, which may be found in multi-chain polypeptides.

In some embodiments, the polypeptides of the disclosure include L-amino acids+glycine, D-amino acids+glycine (which are resistant to L-amino acid-specific proteases in vivo), or a combination of D- and L-amino acids+glycine. Polypeptides of the disclosure may be chemically synthesized or recombinantly expressed.

Polypeptides of the disclosure may include additional residues at the N-terminus, C-terminus, internal to the polypeptide, or a combination thereof, these additional residues are not included in determining the percent identity of the polypeptides of the disclosure relative to the reference polypeptide. Such residues may be any residues suitable for an intended use, including but not limited to tags.

In some embodiments, the term “Tags” refers to a detectable moieties, including but not limited to a fluorescent protein, an antibody epitope tag, a purification tag, a histidine tag, or a linker. In some embodiments, tagged therapeutic agents of the disclosure or tagged ligands of the disclosure comprise a detectable moiety suitable for purposes of purification, to drive localization of the polypeptide, and to add functionality to the polypeptides.

In some embodiments, “Chimeric polypeptide” refers to any polypeptide comprising a first amino acid sequence derived from a first source, which is operably-linked, covalently or non-covalently, to a second amino acid sequence derived from a second source, wherein the first and second source are not the same (two distinct sources). A first source and a second source that are not the same can include two different biological entities, or two different proteins from the same biological entity, or a biological entity and a non-biological entity. A chimeric protein of the disclosure may include a protein derived from at least 2 different biological sources. A biological source may include any non-synthetically produced nucleic acid or amino acid sequence (e.g. a genomic or cDNA sequence, a plasmid or viral vector, a native virion or a mutant or analog of any of the above). A synthetic source may include a protein or nucleic acid sequence produced chemically and not by a biological system (e.g. solid phase synthesis of amino acid sequences). A chimeric protein of the disclosure may include a protein derived from at least 2 different synthetic sources or a protein derived from at least one biological source and at least one synthetic source. A chimeric protein of the disclosure may include a first amino acid sequence derived from a first source, covalently or non-covalently linked to a nucleic acid, derived from any source or a small organic or inorganic molecule derived from any source. The chimeric protein can include a linker molecule between the first and second amino acid sequence or between the first amino acid sequence and the nucleic acid, or between the first amino acid sequence and the small organic or inorganic molecule.

In some embodiments, a “Fragment” of a polypeptide, or a “truncated polypeptide” may refers to an amino acid sequence of a polypeptide that is shorter than a reference sequence. In some embodiments, the reference sequence comprises or consists of a naturally-occurring sequence. In comparison to a reference sequence, the fragment may comprise an N-terminal or a C-terminal deletion (optionally referred to as a truncation). Alternatively, or in addition, in comparison to a reference sequence, the fragment may comprise an internal deletion at any one or more amino acid positions of the polypeptide. Polypeptides of the disclosure may be provided as a fragment or a truncated version of a reference polypeptide. Moreover, all possible fragments and truncated variants of the polypeptides of the disclosure are contemplated in the embodiments provided in this disclosure.

In some embodiments, the term “Functional fragment” refers to a polypeptide fragment that retains a function of the polypeptide. Accordingly, in some embodiments, a functional fragment of a bioactive peptide, such as an enzyme, retains the ability to catalyze a biological action, because the functional fragment comprises a catalytic domain of the enzyme. Polypeptides of the disclosure may be provided as a fragment or a truncated version of a reference polypeptide. Moreover, all possible fragments and truncated variants of the polypeptides of the disclosure are contemplated in the embodiments provided in this disclosure. In some embodiments, a functional fragment of the disclosure retains a function of the polypeptide even if the activity of the functional fragment or the efficacy of the functional fragment is modified when compared to the full-length polypeptide. In some embodiments, a functional fragment of the disclosure retains a function of the polypeptide even if the activity of the functional fragment or the efficacy of the functional fragment is decreased when compared to the full-length polypeptide.

In some embodiments, the term “Functional variant” refers to a modified form of a polypeptide, fragment, or a member of a class of polypeptides, which maintains the function of the polypeptide. In some embodiments, a functional variant of the disclosure retains a function of the polypeptide even if the activity of the functional variant or the efficacy of the functional variant is modified when compared to the unmodified polypeptide. In some embodiments, a functional variant of the disclosure retains a function of the polypeptide even if the activity of the functional variant or the efficacy of the functional variant is decreased when compared to the unmodified polypeptide.

In some embodiments, the term “Amino acid substitution” refers to replacing an amino acid residue present in a parent or reference sequence (e.g., a wild type sequence) with another amino acid residue. An amino acid may be substituted, for example, via chemical peptide synthesis or through recombinant methods known in the art. For example, substituting an amino acid residue with an alternative amino acid residue is conducted by substituting the codon encoding the first amino acid with a codon encoding the second amino acid. The various polypeptide components of the disclosure may be provided with amino acid substitutions.

In some embodiments, “Conservative amino acid substitution” is one in which one amino acid residue is replaced with an amino acid residue having a chemically similar side chain. Families of amino acid residues having similar side chains have been defined in the art, including acidic side chains (e.g., aspartic acid, glutamic acid), basic side chains (e.g., lysine, arginine, histidine), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a chemically similar string that differs in order and/or composition of side chain family members. The various polypeptide components of the disclosure may be provided with conservative amino acid substitutions.

In some embodiments, the term “Non-conservative amino acid substitutions” may refer to those substitutions in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly). The various polypeptide components of the disclosure may be provided with non-conservative amino acid substitutions. The likelihood that one of the foregoing non-conservative substitutions can alter functional properties of the protein is also correlated to the position of the substitution with respect to functionally important regions of the protein: some non-conservative substitutions can accordingly have little or no effect on biological properties. The various polypeptide components of the disclosure may be provided with non-conservative amino acid substitutions that do not significantly alter the functionality of the altered components.

In some embodiments, the terms “Transmembrane element” or “transmembrane domain” may refer to a polypeptide element between the extracellular element and the intracellular element. A portion of the transmembrane element exists within the cell membrane. Chimeric antigen receptors (CARs) of the disclosure include transmembrane elements.

In some embodiments, the terms “Intracellular element” or “intracellular domain” may refer to a polypeptide element that resides on the cytoplasmic side of a cell's cytoplasmic membrane, and transmits a signal into the eukaryotic cell. CARs of the disclosure include intracellular elements. In some embodiments, the cell is a eukaryotic cell.

In some embodiments, the terms “Intracellular signaling element” or “intracellular signaling domain” refers to a portion of the intracellular element that transduces the effector function signal and, which, subsequently directs a cell to perform a specialized function. In some embodiments, the cell is a eukaryotic cell.

In some embodiments, the terms “Extracellular element” or “extracellular element” may refer to the polypeptide element that resides outside of a cell's cytoplasmic membrane. In a CAR-expressing cell, the extracellular element may comprise an antigen binding element of the CAR. In some embodiments, the cell is a eukaryotic cell.

### Sequence Identity and Related Terminology

In some embodiments, two or more sequences are said to be “identical” if they are 100% identical to one another.

In some embodiments, “Identity” refers to the overall monomer conservation between polymeric molecules, e.g., between polypeptide molecules or polynucleotide molecules. “Identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

In some embodiments, when a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

In certain aspects, the percentage identity (% ID) of a first amino acid (or nucleic acid) sequence to a second amino acid (or nucleic acid) sequence is calculated as % ID=100 (Y/Z), where Y is the number of amino acid (or nucleobase) residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

In some embodiments, calculation of the percent identity of two polypeptide sequences, for example, can be performed by aligning the two sequences for optimal comparison purposes. For example, gaps can be introduced in one or both of a first and a second polypeptide sequences for optimal alignment and non-identical sequences can be disregarded for comparison purposes. In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

In some embodiments, the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the European Bioinformatics Institute (EBI) at website ebi.ac.uk/Tools/psa. It will also be appreciated that the final alignment used to calculate percent sequence identity can be curated either automatically or manually.

Suitable software programs are available from various sources, and for alignment of both protein and nucleotide sequences. One suitable program to determine percent sequence identity is b12seq, part of the BLAST suite of program available from the U.S. government's National Center for Biotechnology Information BLAST website (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the EBI. Sequence alignments can be conducted using methods known in the art such as MAFFT, Clustal (ClustalW, Clustal X or Clustal Omega), MUSCLE, etc. Different regions within a single polynucleotide or polypeptide target sequence that aligns with a polynucleotide or polypeptide reference sequence can each have their own percent sequence identity. It is noted that the percent sequence identity value is rounded to the nearest tenth. For example, values from 80.11 to 80.14 are rounded down to 80.1, while values from 80.15 to 80.19 are rounded up to 80.2. It also is noted that the length value will always be an integer.

In some embodiments, “Similarity” refers to the overall relatedness between polymeric molecules, e.g. between polynucleotide molecules and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, for example whether the amino acids are compared, e.g., according to their evolutionary proximity, charge, volume, flexibility, polarity, hydrophobicity, aromaticity, isoelectric point, antigenicity, or combinations thereof.

In some embodiments, the term “linked” may refer to a fusion of a first moiety to a second moiety at the C-terminus or the N-terminus. In some embodiments, the term “linked” may refer to an insertion of the whole first moiety (or the second moiety) into any two points, e.g., amino acids, in the second moiety (or the first moiety, respectively). In some aspects, the first moiety is linked to a second moiety by a peptide bond or a linker. The first moiety can be linked to a second moiety by a phosphodiester bond or a linker. The linker can be a peptide, a polypeptide, a nucleotide, a nucleotide chain or any chemical moiety.

In some embodiments, the term “operably-linked” may refer to two or more elements that are functionally linked within a sequence. In some embodiments, two elements may be functionally linked in a folded protein that are not contiguous with respect to the linear sequence. In some embodiments, two elements may be functionally linked, following expression from a multicistronic sequence. In some embodiments, two elements may be functionally linked because one element can induce or inhibit the expression or function of another, either directly or indirectly.

In some embodiments, the term “non-naturally occurring” may refer to a polypeptide or a polynucleotide sequence that does not exist in nature. In some embodiments, the non-naturally occurring sequence does not exist in nature because the sequence is altered relative to a naturally occurring sequence. In some embodiments, the non-naturally occurring sequence does not exist in nature because it is a combination of two, naturally-occurring, sequences that do not occur together in nature (e.g., chimeric polypeptide). In some embodiments, a non-naturally occurring polypeptide is a chimeric polypeptide. In some embodiments, a polypeptide or a polynucleotide is not naturally occurring because the sequence contains a portion (e.g., a fragment) that cannot be found in nature, i.e., a novel sequence. Any of the polynucleotides described herein may be provided as non-naturally occurring sequences, e.g., having sequences which are altered relative to native sequences or provided as polynucleotides which are linked to other polynucleotides in a manner that does not exist in nature. Any of the polypeptides described herein may be provided as non-naturally occurring sequences, e.g., having sequences which are altered relative to native sequences or provided as polypeptides which are linked to other polypeptides in a manner that does not exist in nature.

### Antibodies and Related Terminology

In some embodiments, the term “Antibody” may refer to various antibody structures, including but not limited to monoclonal antibodies, polyclonal antibodies, and antibody fragments so long as they exhibit the desired antigen-binding activity.

In some embodiments, the term “Antibody fragment” may refer to a molecule other than an intact antibody that includes a portion of an intact antibody that binds the antigen to which the intact antibody binds. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific antibodies formed from antibody fragments. Genes of interest of the disclosure, may for example, include antibody fragments.

In some embodiments, the term “Single chain antibody” (scFv) may refer to an antibody fragment that includes variable regions of heavy (VH) and light (VL) chains, which are linked by a flexible peptide linker.

In some embodiments, the term “Antigen binding molecule” may refer to a molecule that specifically binds an antigenic determinant. Genes of interest of the disclosure, may for example, include antigen binding molecules.

In some embodiments, the term “Antigen” may refer to a molecule that provokes an immune response.

In some embodiments, the term “Chimeric Antigen Receptor” or “CAR” may refer to a fusion protein including antigen recognition moieties and cell-activation elements. Polynucleotides of the disclosure may include genes of interest that produce CARs.

In some embodiments, the term “CAR T cell” or “CAR T lymphocyte” may refer to a T cell containing the capability of producing a CAR polypeptide. For example, a cell that is capable of expressing a CAR is a T cell containing nucleic acid sequences for the expression of the CAR in the cell. Cells of the disclosure may be CAR T-cells.

In some embodiments, the terms “costimulatory element” or “costimulatory signaling domain” or “costimulatory polypeptide” may refer to an intracellular portion of a costimulatory polypeptide. In some embodiments, CARs comprising costimulatory domains demonstrate increased or enhanced T cell expansion, function, persistence and antitumor activity when expressed in a T-cell as compared to a CAR lacking a costimulatory domain. Costimulatory domains may be provided in CARs of the disclosure by incorporating intracellular signaling domains from one or more T cell costimulatory molecules, such as CD28 or 4-1BB.

In some embodiments, a costimulatory polypeptide may comprise a sequence isolated or derived from one or more of the following: a TNF receptor protein, an Immunoglobulin-like protein, a cytokine receptor, an integrin, a signaling lymphocytic activation molecule (SLAM proteins), and an activating natural killer cell receptor. In some embodiments, a costimulatory polypeptide may comprise a sequence isolated or derived from one or more of CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and MyD88.

### Therapeutically Effective and Related Technology

In some embodiments, the term “Therapeutically effective” may refer to the provision of a beneficial effect on the recipient, e.g., providing some alleviation, mitigation, or decrease in a clinical symptom in the subject or an improvement in a clinical state of a subject. Therapeutic effects need not be complete or curative, as long as some benefit is provided to the subject.

In some embodiments, the term “Therapeutically effective amount” may refer to a dose sufficient to impart a therapeutically effective benefit on the recipient. For example, fusion proteins, nucleic acids, vectors, cells, compositions, or pharmaceutical compositions of the disclosure may be administered in a therapeutically effective amount. A subject who has been administered fusion proteins, nucleic acids, vectors, cells, compositions, or pharmaceutical compositions of the disclosure may subsequently be administered a therapeutically effective amount of a small molecule of the disclosure to induce or disrupt dimer formation of the fusion proteins of the disclosure to effect the desired cellular outcome.

### Cell Terminology

In some embodiments, the term “Stem cell” may refer to an undifferentiated or partially differentiated cell that can differentiate into various types of cells and proliferate indefinitely to produce more of the same stem cell.

In some embodiments, the term “Pluripotent stem cell” (PSC) may refer to a cell that can maintain an undifferentiated state indefinitely and can differentiate into most, if not all cells of the body.

In some embodiments, the term “Induced pluripotent stem cell” (iPS or iPSC) may refer to a pluripotent stem cell that can be generated directly from a somatic cell. This includes, but is not limited to, specialized cells such as skin or blood cells derived from an adult.

In some embodiments, the term “Multipotent” may refer to a cell that can develop into more than one cell type but is more limited than a pluripotent cell. For example, adult stem cells and cord blood stem cells may be considered as multipotent.

In some embodiments, the term “Hematopoietic cell” may refer to a cell that arises from a hematopoietic stem cell. This includes, but is not limited to, myeloid progenitor cells, lymphoid progenitor cells, megakaryocytes, erythrocytes, mast cells, myeloblasts, basophils, neutrophils, eosinophils, macrophages, thrombocytes, monocytes, natural killer cells, T lymphocytes, B lymphocytes and plasma cells.

In some embodiments, the term “T-lymphocyte” or T-cell” may refer to a hematopoietic cell that normally develops in the thymus. T-lymphocytes or T-cells include, but are not limited to, natural killer T cells, regulatory T cells, helper T cells, cytotoxic T cells, memory T cells, gamma delta T cells, and mucosal invariant T cells.

In some embodiments, the term “Mesenchyme” may refer to a type of animal tissue included of loose cells embedded in a mesh off proteins and fluid, i.e., the extracellular matrix. Mesenchyme directly gives rise to most of the body's connective tissues including bones, cartilage, lymphatic system, and circulatory system.

In some embodiments, the term “Mesenchymal cell” may refer to a cell that is derived from a mesenchymal tissue. In some cases, cells of the disclosure may be mesenchymal cells.

In some embodiments, the term “Mesenchymal stromal cell” (MSC) may refer to a spindle shaped plastic-adherent cell isolated from bone marrow, adipose, and other tissue sources, with multipotent differentiation capacity in vitro. For example, a mesenchymal stromal cell can differentiate into osteoblasts (bone cells), chondrocytes (cartilage cells), myocytes (muscle cells), and adipocytes (fat cells which give rise to marrow adipose tissue). The term mesenchymal stromal cell is suggested in the scientific literature to replace the term “mesenchymal stem cell”. In some cases, cells of the disclosure may be mesenchymal stromal cells.

In some embodiments of the disclosure, an “autologous cell” is a cell obtained from the same individual to whom it may be administered as a therapy (the cell is autologous to the subject). Autologous cells of the disclosure include, but are not limited to, hematopoietic cells and stem cells, such as hematopoietic stem cells.

In some embodiments of the disclosure, an allogeneic cell is a cell obtained from an individual who is not the intended recipient of the cell as a therapy (the cell is allogeneic to the subject). Allogeneic cells of the disclosure may be selected from immunologically compatible donors with respect to the subject of the methods of the disclosure. Allogeneic cells of the disclosure may be modified to produce “universal” allogeneic cells, suitable for administration to any subject without unintended immunogenicity. Allogeneic cells of the disclosure include, but are not limited to, hematopoietic cells and stem cells, such as hematopoietic stem cells.

In some embodiments, the terms “Transfect” or “transform” or “transduce” may refer to a process by which exogenous nucleic acid is transferred or introduced into a cell or a host cell. A “transfected” or “transformed” or “transduced” cell is one which has been transfected, transformed or transduced with exogenous nucleic acid or progeny of the cell.

In some embodiments, the term “Cell therapy” may refer to the delivery of a cell or cells into a recipient for therapeutic purposes.

### Small Molecule Terminology

In some embodiments, the term “Analog” may refer to a chemically modified form of a compound, or member of a class of compounds, described herein which maintains the binding properties of the compound or class. For example, an analog of danoprevir would include chemically modified forms of danoprevir that retains the ability to bind DNCR2 and NS3a as described herein.

In some embodiments, the term “prodrug”, may include any covalently bonded carriers which release a small molecule of the disclosure in vivo when such prodrug is administered to a patient. Prodrugs of the disclosure may be prepared by modifying functional groups present in the compound in such a way that the modifications are cleaved, either in routine manipulation or in vivo, to the parent compound. The transformation in vivo may be, for example, as the result of some metabolic process, such as chemical or enzymatic hydrolysis of a carboxylic, phosphoric or sulphate ester, or reduction or oxidation of a susceptible functionality. Prodrugs within the scope of the disclosure include compounds wherein a hydroxy, amino, or sulfhydryl group is bonded to any group that, when the prodrug of the disclosure is administered to a mammalian subject, it cleaves to form a free hydroxyl, free amino, or free sulfhydryl group, respectively. Functional groups that may be rapidly transformed, by metabolic cleavage, in vivo form a class of groups reactive with the carboxyl group of the compounds of this disclosure. They include, but are not limited to, such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkysilyl (such as trimethyl- and triethysilyl), monoesters formed with dicarboxylic acids (such as succinyl), and the like. The small molecules of the disclosure may be administered as prodrugs. The small molecules of the disclosure may be administered to a subject as a prodrugs. A therapeutically effective amount of such a prodrug of the disclosure may be administered. The prodrug may be administered contemporaneously with the administration of the polynucleotides, gene therapy vectors or cells of the disclosure or following the administration of the polynucleotides, gene therapy vectors or cells of the disclosure.

### Formulations and Related Technology

In some embodiments, the term “Pharmaceutically acceptable” may refer to compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio. For example, the small molecules, polynucleotides, polypeptides, gene therapy vectors or cells of the disclosure may be administered as part of a composition together with other pharmaceutically acceptable components, including pharmaceutically acceptable carriers.

In some embodiments, the term “Pharmaceutically acceptable salts” may refer to derivatives of the small molecules of the disclosure wherein the specified compound is converted to an acid or base salt thereof. Such pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluensulfonic, methanesulfonic, ethane dislfonic, oxalic, isethionic, and the like. For example, the small molecules of the disclosure may be provided as pharmaceutically acceptable salts.

In some embodiments, the term “Controlled release” may refer to part or all of a dosage form that can release one or more active pharmaceutical agents over a prolonged period of time (i.e., over a period of more than 1 hour). The characteristic of controlled release (CR) may also be referred to as sustained release (SR), prolonged release (PR), or extended release (ER). When used in association with the dissolution profiles discussed herein, the term “controlled release” refers to that portion of a dosage form according to the disclosure that delivers active agent over a period of time greater than 1 hour. For example, the small molecules of the disclosure may be administered in a controlled release composition.

In some embodiments, the term “Immediate release” may refer to part or all of a dosage form that releases active agent substantially immediately upon contact with gastric juices and that results in substantially complete dissolution within about 1 hour. The characteristic of immediate release (IR) may also be referred to as instant release (IR). When used in association with the dissolution profiles discussed herein, the term “immediate release” refers to that portion of a dosage form according to the disclosure that delivers active agent over a period of time less than 1 hour. The small molecules of the disclosure may be administered in an immediate release composition.

In some embodiments, the term “Excipients” may refer to pharmacologically inert ingredients that are not active in the body. See, for example, Hancock, B. C., Moss, G. P., & amp; Goldfarb, D. J. (2020). Handbook of pharmaceutical excipients. London. Pharmaceutical Press, the entire disclosure of which is incorporated herein by reference. The small molecules of the disclosure may be mixed with pharmaceutically acceptable carriers, diluents, adjuvants, excipients, or vehicles, such as preserving agents, fillers, polymers, disintegrating agents, glidants, wetting agents, emulsifying agents, suspending agents, sweetening agents, flavoring agents, perfuming agents, lubricating agents, acidifying agents, and dispensing agents, depending on the nature of the mode of administration and dosage forms. Such ingredients, including pharmaceutically acceptable carriers and excipients that may be used to formulate oral dosage forms. Pharmaceutically acceptable carriers include water, ethanol, polyols, vegetable oils, fats, waxes polymers, including gel forming and non-gel forming polymers, and suitable mixtures thereof. Examples of excipients include starch, pregelatinized starch, Avicel, lactose, milk sugar, sodium citrate, calcium carbonate, dicalcium phosphate, and lake blend. Examples of disintegrating agents include starch, alginic acids, and certain complex silicates. Examples of lubricants include magnesium stearate, sodium lauryl sulphate, talc, as well as high molecular weight polyethylene glycols. For example, the small molecules, polynucleotides, polypeptides, gene therapy vectors or cells of the disclosure may be provided and administered in compositions that include pharmaceutically acceptable excipients.

### General Terminology

As used throughout the disclosure, the term “Subject” includes any mammal, including without limitation, humans. In some embodiments, the subject is a human. In some embodiments, the subject is a neonate, an infant, a toddler, a child, a teenager, an adult, a senior, a centenarian. In some embodiments, the subject has at least 1, 2, or 3 X chromosomes. In some embodiments, the subject has at least 1 or 2 Y chromosomes. In some embodiments, the subject is diagnosed with a disease or disorder of the disclosure, or otherwise, in need of treatment of the disclosure. In some embodiments, the subject is at risk of developing a disease or disorder of the disclosure, or otherwise, in need of preventing a disease or disorder of the disclosure.

In some embodiments, the subject is a non-human primate. In some embodiments, the subject is a mammal, including, but not limited to, livestock, a horse, a dog, a cat, a pig, a rabbit, a guinea pig, a rodent, a rat, a gerbil, and a mouse. In some embodiments, the subject is a non-primate mammal and the subject is genetically-modified.

“A”, “an” and “the” include their plural forms unless the context clearly dictates otherwise.

“And” is used interchangeably with “or” unless expressly stated otherwise.

“And/or” is to be taken as specific disclosure of each of the two specified features or components with or without the other. Thus, “and/or” as used in a phrase such as “A and/or B,” includes “A and B,” “A or B,” “A” (alone), and “B” (alone). Likewise, “and/or,” as used in a phrase such as “A, B, and/or C,” is intended to encompass each of the following aspects: A, B, and C; A, B, or C; A or C; A or B; B or C; A and C; A and B; B and C; A (alone); B (alone); and C (alone).

In some embodiments, the term “About” means approximately, roughly, around, or in the regions of. When “about” is used with a numerical range, it may modify that range by extending the boundaries above and below the numerical values set forth.

In some embodiments, numeric ranges are inclusive of the numbers defining the range. Where a range of values is stated, each intervening integer value, and each fraction thereof, between the recited upper and lower limits of that range is also specifically disclosed, as is each subrange between such values. The upper and lower limits of any range can independently be included in or excluded from the range, and each range where either, neither or both limits are included is also encompassed within the disclosure. Thus, ranges are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly stated, it is to be understood that values which are about the same quantity or amount as the stated value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Unless the context requires otherwise, throughout the description and the claims, the words ‘include’, ‘including’, and the like are to be construed in an inclusive sense as opposed to an exclusive or exhaustive sense; that is to say, in the sense of “including, but not limited to”.

Singular or plural words also include the plural and singular number, respectively. Thus, for example, where the specification describes a gene of interest, the disclosure includes polynucleotides with a single gene of interest or multiple genes of interest.

“Above,” and “below” and words of similar import refer to this application as a whole and not to any particular portions of the application.

“Set” includes sets of one or more elements or objects.

Units, prefixes, and symbols are denoted in their Systeme International de Unites (SI) accepted form.

Headings are included herein for reference and to aid in locating the various sections. These headings are not intended to limit the scope of the concepts described with respect to the headings. Such concepts may have applicability throughout the present specification.

Although the disclosure is described in some detail by way of illustration and example for purposes of clarity and understanding, it will be apparent to those skilled in the art that certain changes and modifications may be practiced. Reference to “the disclosure” or the like is intended as a reference to any of a wide variety of embodiments of, or aspects of, the disclosure, and not as limiting the disclosure to a single embodiment or aspect. As used throughout the disclosure, the terms “aspect” and “embodiment” are interchangeable. Features discussed in the context of “certain”, “some”, or “other” aspects or embodiments of the disclosure may be found in any embodiment of the disclosure, however, in these instances, the feature may be considered a preferred feature in these highlighted embodiments.

The description and examples should not be construed as limiting the scope of the disclosure to the embodiments and examples described herein, but rather as encompassing all modifications and alternatives falling within the true scope and spirit of the disclosure.

### Small Molecule-Regulated Cell Signaling Expression System

The disclosure provides a small molecule-regulated cell signaling expression system for controlling receptor-mediated cell activation and signaling. The system generally includes a polynucleotide set that includes a first polynucleotide encoding a first fusion protein that includes a first signaling domain and first dimerization polypeptide, and a second polynucleotide encoding a second fusion protein that includes a second signaling domain and a second dimerization polypeptide, wherein the interaction of the first and second dimerization polypeptides is mediated by the presence of a small molecule.

In some embodiments, the small molecule mediates binding of the first and second dimerization polypeptides.

In some embodiments, the small molecule disrupts binding of the first and second dimerization polypeptides.

In some embodiments, a first small molecule mediates binding of the first and second dimerization polypeptides, and a second small molecule disrupts binding of the first and second dimerization polypeptides, wherein the second small molecule can mediate binding of a third dimerization polypeptide to the first dimerization polypeptide, and the third dimerization polypeptide is linked to a third signaling domain.

In some embodiments, the system includes a polynucleotide set wherein the signaling domains linked to the dimerization polypeptides are part of a chimeric antigen receptor (CAR), and wherein administration of the small molecule modulates a T cell signaling function.

In some embodiments, the system includes a polynucleotide set encoding a first fusion protein that includes a first dimerization polypeptide linked to a cytoplasmic end of a transmembrane CAR lacking a CD3ζ domain, and the second fusion protein includes a second dimerization polypeptide linked to a CD3ζ domain, and wherein administration of the small molecule mediates CAR T cell signaling.

In some embodiments, the system includes a polynucleotide set encoding a first fusion protein that includes NS3a linked to a cytoplasmic end of a transmembrane CAR lacking a CD3ζ domain, and the second fusion protein includes ANR linked to a CD3ζ domain, and wherein administration of the small molecule down-regulates CAR T cell signaling.

In some embodiments, the system includes a polynucleotide set encoding a first fusion protein that includes NS3a linked to a cytoplasmic end of a transmembrane CAR lacking a CD3ζ domain, and the second fusion protein includes DNCR2 (or designed variants thereof) or GNCR1 (or designed variants thereof) linked to a CD3ζ domain, and wherein administration of the small molecule activates CAR T cell signaling.

In some embodiments, the system includes a polynucleotide set wherein the first and second signaling domains are killing domains, and wherein administration of the small molecule induces cell death.

The small molecule-regulated cell signaling system of the disclosure is useful for regulating the activity of a range of target molecules (e.g., signaling proteins) in various synthetic biology applications, such as therapeutic applications.

In one embodiment, the small molecule-regulated cell signaling system of the disclosure may be used in a cell therapy application to program a population of cells for performing and/or modulating a therapeutic function.

In one aspect, the small molecule-regulated cell signaling system of the disclosure may be used to regulate T cell receptor (TCR) and/or chimeric antigen receptor (CAR) cells used in cancer therapy. More specifically, the small molecule-regulated cell signaling system of the disclosure may be used to down-regulate CARs and/or TCRs and inhibit excessive CAR/TCR signaling that may result in undesirable exhaustion phenotypes observed in many existing T-cell therapies.

In one embodiment, the small molecule-regulated cell signaling system of the disclosure may be used in gene therapy applications.

### Small Molecule Regulated Dimers

The disclosure makes use of small molecule-regulated polypeptide dimers to colocalize signaling domains and thereby regulate a signaling function. For example, the dimers may colocalize an extracellular signaling domain and a cytoplasmic (intracellular) signaling domain and thereby regulate the activity of a signaling molecule of interest. Interaction of the dimerization polypeptides to form a dimerization complex can be regulated by the presence of the small molecule.

The dimers may be used to colocalize signaling domains of a transmembrane receptor. For example, the dimers may be used to colocalize signaling domains of a transmembrane CAR that includes:

(i) a first fusion protein that includes a first dimerization polypeptide linked to an extracellular signaling domain lacking a cytoplasmic signaling domain; and
(ii) a second fusion protein that includes a second dimerization polypeptide linked to the corresponding cytoplasmic signaling domain, wherein interaction of the first and second dimerization polypeptides is mediated by the presence of a small molecule.

The first and second dimerization polypeptides may be selected so that the interaction of the first and second dimerization polypeptides is mediated by the presence of the small molecule. In some cases, the small molecule may mediate assembly of the dimer. In other cases, the small molecule may mediate disassembly of the dimer. In yet other cases, a first small molecule may mediate assembly of the dimer while a second small molecule may displace the first small molecule and thereby mediate disassembly of the dimer. In still other cases, a first small molecule may mediate assembly of the dimer while a second small molecule may out-compete the first small molecule and recruit a third dimerization polypeptide for dimerization.

A small molecule regulated polypeptide dimer may include the hepatitis C virus protease NS3a/4a protein (hereafter referred to as NS3a) or a modification thereof as a first dimerization polypeptide and a “reader” protein as a second dimerization polypeptide. The reader protein may, for example, be selected to recognize a specific drug-bound state of the NS3a protein. NS3a proteins and NS3a reader proteins have been described in Baker et al., International Patent Publication WO2020117778, entitled “Reagents and Methods for Controlling Protein Function and Interaction,” published on Jun. 11, 2020, which is incorporated herein by reference in its entirety.

NS3a can integrate multiple drug inputs and translate the drug inputs into diverse outputs using different engineered reader proteins as dimerization partners. NS3a proteins and pleiotropic response outputs from danoprevir/NS3a complex readers, grazoprevir/NS3a complex readers, and ANR/NS3a complex readers have been described in Foight, G. W., et al., Nature Biotechnology (2019) 37:1209-1216; Cunningham-Bryant, D. et al., Journal of the American Chemical Society (2019) 141: 3352-3355; and Kigler, J., et al., Journal of Biological Chemistry (2012) 287:39224-39232, which are incorporated herein by reference in their entireties.

In one example, the dimers may be used to colocalize signaling domains of a transmembrane CAR that includes:

(i) a first fusion protein that includes an NS3a polypeptide and an extracellular signaling domain lacking a cytoplasmic signaling domain; and
(ii) a second fusion protein that includes a reader polypeptide and the corresponding cytoplasmic signaling domain, wherein interaction between the NS3a and reader binding partners may be controlled by the presence of a small molecule drug.

In some embodiments, the reader selected for the dimer is a danoprevir/NS3 complex reader (DNCR) polypeptide (or minimized/modified variants thereof) designed to recognize and bind NS3a in the presence of the small molecule drug danoprevir, thereby providing a drug-inducible (“on-switch”) system. In one example the DNCR polypeptide is DNCR2. See Foight, G. W., et al., Nature Biotechnology (2019) 37:1209-1216.

In some embodiments, the reader selected for the dimer is a grazoprevir/NS3 complex reader (GNCR) polypeptide (or minimized/modified variants thereof) designed to recognize and bind NS3a in the presence of the small molecule drug grazoprevir, thereby providing a drug-inducible (“on-switch”) system. In one example, the GNCR protein is GNCR1. See Foight, G. W., et al., Nature Biotechnology (2019) 37:1209-1216.

In some embodiments, the reader selected for the dimer is an apoNS3a complex reader (ANR) peptide (or minimized/modified variants thereof). ANR forms a basal complex with NS3a, which is disrupted by NS3a-targeting drugs, thereby providing a drug-disreputable system. See Cunningham-Bryant, D., et al., Journal of the American Chemical Society (2019) 141:3352-3355, Kügler, J., et al., Journal of Biological Chemistry (2012) 287:39224-39232, and Foight, G. W., et al., Nature Biotechnology (2019) 37:1209-1216.

### Chimeric Antigen Receptors

A signaling molecule of interest may include chimeric antigen receptors (CARs). CARs can be fusion proteins including an extracellular element that is an antigen binding element, a transmembrane element that anchors the receptor to the cell membrane and at least one intracellular element. These CAR elements are known in the art, for example as described in patent application US20140242701, entitled “Chimeric Antigen Receptors”, published on Aug. 28, 2014, which is incorporated by reference in its entirety. The CAR can be a recombinant polypeptide expressed from a polynucleotide including at least an extracellular antigen binding element, a transmembrane element and an intracellular (cytoplasmic) signaling element including a functional signaling element derived from a stimulatory molecule.

The stimulatory molecule may, for example, be the zeta chain associated with the T cell receptor complex.

The cytoplasmic signaling element may, for example, include one or more functional signaling elements derived from at least one costimulatory molecule.

The costimulatory molecule may, for example, be selected from 4-1BB (i.e., CD137), CD27 and/or CD28.

The CAR may be a chimeric fusion protein including an extracellular antigen binding element, a transmembrane element and an intracellular signaling element including a functional signaling element derived from a stimulatory molecule.

The CAR may include a chimeric fusion protein including an extracellular antigen binding element, a transmembrane element and an intracellular signaling element including a functional signaling element derived from a costimulatory molecule, and a functional signaling element derived from a stimulatory molecule.

The CAR may be a chimeric fusion protein including an extracellular antigen binding element, a transmembrane element, and an intracellular signaling element including two functional signaling elements derived from one or more costimulatory molecule(s) and a functional signaling element derived from a stimulatory molecule.

The CAR may include a chimeric fusion protein including an extracellular antigen binding element, a transmembrane element, and an intracellular signaling element including at least two functional signaling elements derived from one or more costimulatory molecule(s) and a functional signaling element derived from a stimulatory molecule.

The CAR may include an optional leader sequence at the amino-terminus (N-term) of the CAR fusion protein. The CAR may further include a leader sequence at the N-terminus of the extracellular antigen binding element, wherein the leader sequence is optionally cleaved from the antigen recognition element (e.g., a scFv) during cellular processing and localization of the CAR to the cellular membrane.

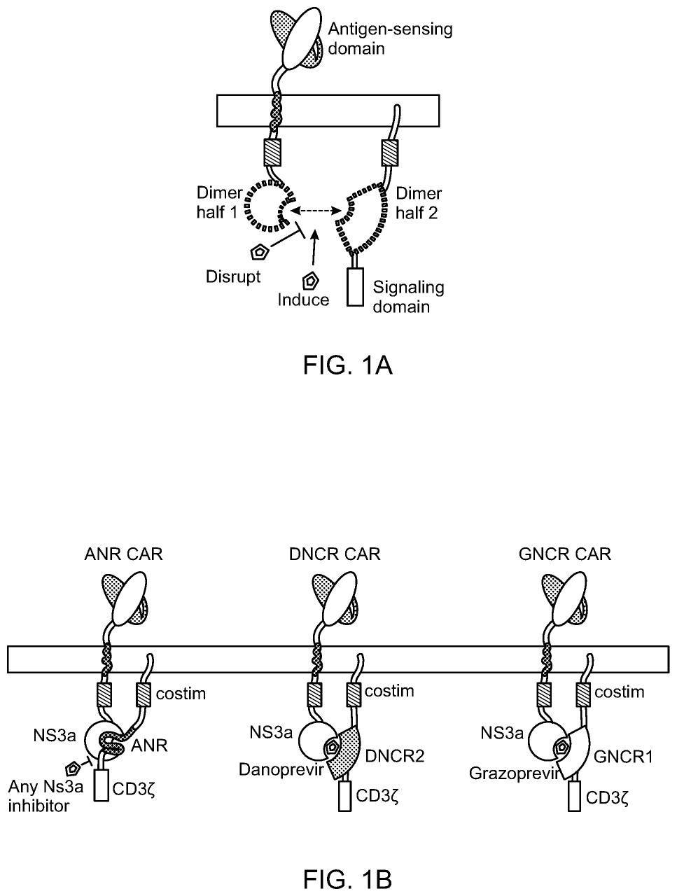

FIG. 1A is a schematic diagram of a generalized architecture of a split chimeric antigen receptor (CAR) that can be regulated by small molecule drugs to disrupt or induce a cellular signaling activity. The figure illustrates expressed components 110 of the system, wherein a first fusion protein includes an extracellular element that is an antigen binding or “antigen-sensing” domain fused to a first dimerization polypeptide (“dimer half 1”) and a second fusion protein includes a cytoplasmic signaling domain fused to a second dimerization polypeptide (“dimer half 2”). The interaction of the first dimerization polypeptide and the second dimerization polypeptide can be regulated by small molecule drugs that disrupt (i.e., turn OFF) or induce (i.e., turn ON) signaling activity.

In some embodiments, a small molecule-regulated CAR expression system may be an “off-switch” CAR. A chemically disruptable dimer formed by the peptide apo-NS3a reader (ANR) and NS3a has been described. See Cunningham-Bryant, D., et al., Journal of the American Chemical Society (2019) 141: 3352-3355, which is incorporated herein by reference in its entirety.

In some embodiments, a small molecule-regulated CAR expression system may be an “on-switch CAR. Two “on-switch” CARs using the designed chemically induced dimers danoprevir-NS3a complex reader (DNCR)/danoprevir/NS3a and grazoprevir-NS3a complex reader GNCR1/grazoprevir/NS3a have been described. See Foight, G. W., et al., Nature Biotechnology (2019) 37: 1209-1212, which is incorporated herein by reference in its entirety.

FIG. 1B is a schematic diagram 120 of an off-switch ANR CAR, an on-switch DNCR CAR, and an on-switch GNCR CAR. The figure illustrates expressed components of an ANR CAR 125, a DNCR CAR 130, and a GNCR CAR 135. ANR CAR 125 is ON by default and can be turned OFF by any NS3a inhibitor, including danoprevir or grazoprevir. DNCR CAR 130 and GNCR CAR 135 are OFF by default and can be turned on by the addition of danoprevir or grazoprevir, respectively.

### Killing Domains

A signaling molecule of interest may include a death or killing domain that can initiate signaling cascades leading to cell death. The utility of cell and gene therapies can be enhanced by safety mechanisms that enable specific induction of cell death in the therapeutic cells in the event of adverse reactions in patients. This concept of using cell death as a safety mechanism has been previously demonstrated by induction of apoptosis with caspase-9 fused to the small molecule inducible homodimer FKBP-F36V (iCasp9), which is dimerized by AP1903 (rimiducid) (di Stais, et al., New England Journal of Medicine (2011) 365: 1673-1683, which is incorporated herein by reference in its entirety). Chemically inducible heterodimers can also be used to initiate oligomerization of caspase-9, as has been demonstrated with the FKBP/rapamycin/FRB dimer (rapaCasp9) (Stavrou, M., et al., Molecular Therapy (2018) 26: 1266-1276, which is incorporated herein by reference in its entirety).

The disclosure makes use of small molecule-regulated dimer systems DNCR/danoprevir/NS3a and GNCR/grazoprevir/NS3a to activate cell death through drug-induced homo-oligomerization of killing domains. The small molecule-regulated dimer systems DNCR/danoprevir/NS3a and GNCR/grazoprevir/NS3a systems can be used to regulate any killing domain that is activated by homo-oligomerization (e.g., caspase-9, caspase-1, caspase-4, RIPK1, RIPK3).

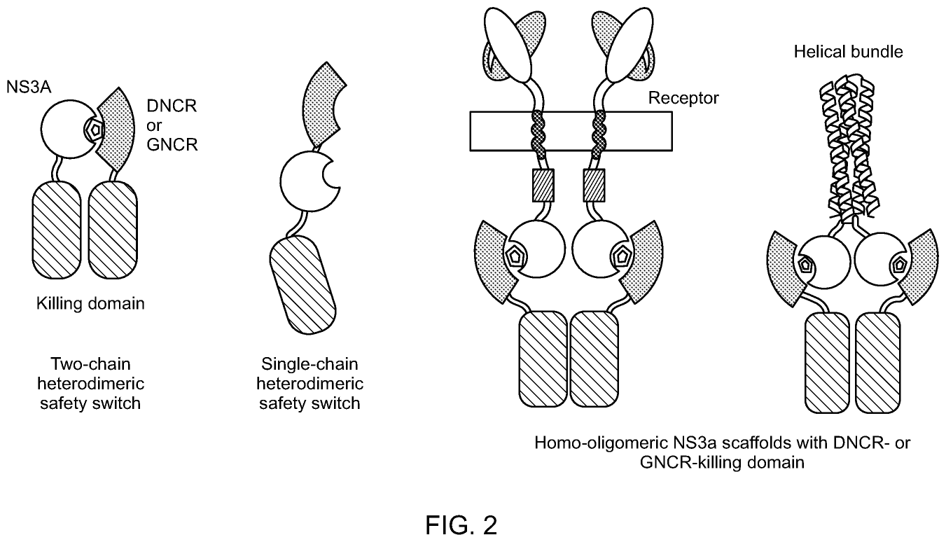

FIG. 2 is a schematic diagram 200 of designs to induce homo-oligomerization of killing domains (e.g., caspase-9) in response to small molecule drugs. The figure illustrates expressed components of a two-chain heterodimeric safety switch 210, a single-chain heterodimeric safety switch 215, and homo-oligomeric NS3a scaffold safety switches 220a and 220b. Two-chain heterodimeric safety switch 210 includes each half of the small molecule induced dimer, NS3a and DNCR or GNCR, fused to the killing domain. In one example NS3a-killing domain binds to DNCR-killing domain only in the presence of the small molecule drug danoprevir. Single-chain heterodimeric safety switch 215 includes both halves of the small molecule induced dimer linked in a single construct with the killing domain such that addition of the small molecule drug induces multimerization, i.e., templated homo-oligomerization. Homo-oligomeric NS3a scaffold safety switches 220a and 220b include the NS3a dimerization polypeptide linked to a homo-dimeric receptor such as a CAR (safety switch 220a) or a homo-oligomeric helical bundle (safety switch 220b) and the other dimerization polypeptide fused the killing domain such that addition of the small molecule drug mediates oligomerization of the killing domain. Design of protein homo-oligomeric helical bundles has been described in Boyken, D. E et al., Science (2016) 352: 680-687, which is incorporated herein by reference in its entirety.

### 1.1.1. Polynucleotides

The system of the disclosure generally includes a polynucleotide set that includes a first polynucleotide encoding a first fusion protein and a second polynucleotide encoding a second fusion protein.

In some embodiments, the polynucleotide set includes a polynucleotide component that may include:

(i) a first polynucleotide encoding a first fusion protein that includes a first signaling domain and first dimerization polypeptide,
(ii) a second polynucleotide encoding a second fusion protein that includes a second signaling domain and a second dimerization polypeptide,
(iii) one or more promoter sequences operatively linked to the first and/or second polynucleotides,
(iv) an optional separation element that includes a polynucleotide sequence that prevents fusion of the first fusion protein and the second fusion protein, and
(v) one or more optional regulatory sequences, wherein the first and second polynucleotides, promoter sequence(s), and optional separation element and regulatory elements are configured for expression and regulated control of a signaling function of interest.

The polynucleotides encoding the first and second fusion proteins, promoter sequence(s), and optional separation element and regulatory sequences may be configured in a vector backbone for expression and regulated control of a signaling function of interest.

In various embodiments, the polynucleotide component encoding the fusion proteins may include a polynucleotide sequence encoding a separation element separating the fusion proteins.

In some embodiments, the separation element may include a ribosomal skipping sequence selected from the group consisting of P2a and T2a.

In some embodiments, the separation element may include a polynucleotide sequence that includes at least two ribosomal skipping sequences selected from the group consisting of T2a-RFP-P2a, P2a-T2a, and T2a-P2a.

In some embodiments, the separation element may include an internal ribosome entry site (IRES).

In some embodiments, the separation element may include a constitutive promoter sequence.

In some embodiments, the polynucleotide component that includes the first and second polynucleotides encodes one or more constitutive promoter sequences operatively linked to the first and/or second polynucleotides.

In various embodiments, the constitutive promoter sequence may include a constitutive promoter sequence selected from the group consisting of MND, hPGK, CMV, CAG, SFFV, EF1alpha, UBC, and CD43.

In some embodiments, the first and/or second polynucleotides may encode one or more optional regulatory sequences selected from the group consisting of polyA.

In some embodiments, the polynucleotides encoding the first and/or second fusion proteins further include a flexible linker sequence separating the signaling domain and dimerization polypeptides.

In some embodiments, the first or second polynucleotide encodes an NS3a dimerization polypeptide (or designed variant thereof) and the other of the first or second polynucleotide encodes a corresponding reader dimerization polypeptide selected from the group consisting of DNCR2 (or designed variants thereof, including but not limited to DNCR2_1 through DNCR2_34, and DNCR2-3rep) or GNCR1 (or designed variants thereof, including but not limited to GNCR1-3rep, G33, and G38).

In some embodiments, the polynucleotide set encodes a first fusion protein that includes NS3a dimerization polypeptide linked to the cytoplasmic end of a transmembrane CAR lacking a CD3ζ domain and a second fusion protein that includes an ANR dimerization polypeptide linked to a CD3ζ domain, wherein the small molecule down-regulates CAR T cell signaling.

In some embodiments, the polynucleotide set encodes a first fusion protein that includes NS3a dimerization polypeptide linked to the cytoplasmic end of a transmembrane CAR lacking a CD3ζ domain and a second fusion protein that includes DNCR2 (or designed variants thereof) or GNCR1 (or designed variants thereof) linked to a CD3ζ domain, wherein the small molecule activates CAR T cell signaling.

In some embodiments, the polynucleotide encoding the first and/or second fusion protein further encodes a transmembrane domain and/or a costimulatory domain.

In some embodiments, the first and second polynucleotides encode signaling domains that include killing domains, wherein the administration of the small molecule induces cell death.

In some embodiments, the polynucleotide encoding the first and/or second fusion protein includes a killing domain selected from the group consisting of: caspase-9, caspase-1, caspase-4, RIPK1, or RIPK3, or variants thereof.

In some embodiments, the polynucleotide set encodes a first signaling domain that includes a catalytic domain of caspase-9 (or a variant thereof) and a second signaling domain that includes a catalytic domain of capsase-9 (or a variant thereof).

In some embodiments, the polynucleotide set encodes a first dimerization polypeptide linked to a signaling domain that includes a catalytic domain of caspase-9 (or a variant thereof) and a second dimerization polypeptide is linked to a homo-oligomerization domain.

In some embodiments, the polynucleotide set encodes a first dimerization polypeptide selected from the group consisting of DNCR2 (or designed variants thereof, including but not limited to DNCR2_1 through DNCR2_34, and DNCR2-3rep) or GNCR1 (or variants thereof, including but not limited to GNCR1-3rep, G33, and G38) linked to a killing domain and the second dimerization polypeptide is NS3a and is linked to a homo-oligomerization domain selected from the group of: dimer-NS3aH1 (SEQ ID NO: 42), trimer-NS3aH1 (SEQ ID NO: 45), pentamer-NS3aH1 (SEQ ID NO: 44), hexamer-NS3a (SEQ ID NO; 43), or variants thereof.

In some embodiments, the polynucleotide set encodes a first dimerization polypeptide selected from the group consisting of DNCR2 (or designed variants thereof, including but not limited to DNCR2_1 through DNCR2_34, and DNCR2-3rep) or GNCR1 (or variants thereof, including but not limited to GNCR1-3rep, G33, and G38) linked to a killing domain and the second dimerization polypeptide is NS3a and is linked to a CAR or other homo-oligomeric receptor.

### Vectors and Vector Configurations

The polynucleotides of the disclosure may be provided as part of a vector. Examples of suitable vectors include expression vectors, viral vectors, and plasmid vectors. Expression vectors can include plasmids, phagemids, viruses, and derivatives thereof.

In some aspects, the viral vectors may include polynucleotides encoding gene editing polypeptides, such as polypeptides useful for implementation of gene editing techniques. Examples of such gene editing techniques include RNA/DNA guided endonucleases (e.g., CRISPR (clustered regularly interspaced short palindromic repeats)), TALEN (transcription activator-like effector nucleases), ZFN (zinc finger nucleases), recombinase, meganucleases, or viral integration.

In some aspects, the polynucleotides of the disclosure may be provided as part of a homology directed repair (HDR) vector. A homology directed repair mechanism may be used to integrate a polynucleotide set into a chromosome. Examples of mechanisms that may be used to integrate a polynucleotide set into a chromosome include sequence-specific nucleases such as transposase, CRISPR/Cas9, ZF nucleases, TALE nucleases, recombinases, and other homologous recombination targeting vectors known in the art.

Vector components generally include, but are not limited to, one or more of the following: a signal sequence, an origin of replication, one or more marker genes, an enhancer element, a promoter, and a transcription termination sequence. A vector for use in a eukaryotic host cell may also encode a signal sequence or other polypeptide having a specific cleavage site at the N-terminus of the mature protein or polypeptide of interest. The signal sequence selected is preferably one that is recognized and processed (i.e., cleaved by a signal peptidase) by the host cell. In mammalian cell expression, mammalian signal sequences as well as viral secretory leaders may be used. Expression vectors used in eukaryotic host cells will typically also contain sequences necessary for the termination of transcription and for stabilizing the mRNA. Such sequences are commonly available from the 5′ and, occasionally 3′, untranslated regions of eukaryotic or viral DNAs or cDNAs. One useful transcription termination component is the bovine growth hormone polyadenylation region.

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

The polynucleotides of the disclosure may in some cases be provided as part of a single vector. The polynucleotides of the disclosure may be provided as part of a set of at least two vectors; a first vector including the first polynucleotide and a second vector including the second polynucleotide. In some cases, the polynucleotides of the disclosure may be provided as a set of three vectors: a first vector including the first polynucleotide, a second vector including the second polynucleotide, and a third vector including a third polynucleotide.

Examples of vectors suitable for use with the polynucleotides of the disclosure include adenoviral vectors, retroviral vectors such as lentiviral vectors, baculoviral vectors, Epstein Barr viral vectors, papovaviral vectors, vaccinia viral vectors, herpes simplex viral vectors, adeno associated virus (AAV) vectors, and transposon vectors. The polynucleotides of the disclosure may be provided as part of a homology directed repair vector.

Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., Gene Transfer and Expression, A Laboratory Manual, W.H. Freeman Co., New York (1990) and Murry, E. I, Methods in Molecular Biology, Vol. 7, Humana Press, Inc., Cliffton, N.J. (1991).

In some aspects, the virus is an adeno-associated virus, a double-stranded DNA virus. The adeno-associated virus can be engineered to be replication-deficient and is capable of infecting a wide range of cell types and species. It further has advantages such as heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well-known to those of skill in the art. See, e.g., Sambrook et al, Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Some commonly used transposon systems include piggyBAC™, Tol2, and Sleeping Beauty™ (See, e.g., Balasubramanian et al. Comparison of three transposons for the generation of highly productive recombinant CHO cell pools and cell lines. Biotechnology and Bioengineering (2015) 113, p 1234-1243.). Other plasmids are well-known to those of ordinary skill in the art. Additionally, plasmids may be custom designed using standard molecular biology techniques to remove and/or add specific fragments of DNA.

The disclosure provides a polynucleotide set that includes the following as part of one or more vectors:

(i) a first polynucleotide encoding a first fusion protein that includes a first dimerization polypeptide, and
(ii) a second polynucleotide encoding a second fusion protein that includes a second corresponding dimerization polypeptide, wherein:
(iii) the first or second fusion protein further includes a first signaling domain; and (iv) the other of the first or second fusion protein further includes a second signaling domain; and
(v) interaction of the first and second dimerization polypeptides in response to a small molecule regulator effectively regulates a signaling function.

In some embodiments, the disclosure provides a polynucleotide set that includes the following as part of one or more vectors:

(i) a first polynucleotide encoding a first fusion protein that includes a first dimerization polypeptide, and
(ii) a second polynucleotide encoding a second fusion protein that includes a corresponding second dimerization polypeptide, wherein:
(iii) the first or second fusion protein further includes an extracellular signaling domain; and (iv) the other of the first or second fusion protein further includes a cytoplasmic signaling domain; and
(v) interaction of the first and second dimerization polypeptides in response to a small molecule regulator effectively regulates a signaling function.

In some embodiments, the disclosure provides a polynucleotide set that includes the following as part of one or more vectors:

(i) a first polynucleotide encoding a first fusion protein that includes an NS3a dimerization polypeptide and a first signaling domain; and
(ii) a second polynucleotide encoding a second fusion protein that includes a reader dimerization polypeptide and a second signaling domain, wherein interaction of the NS3a and reader dimerization polypeptides in response to a small molecule regulator effectively regulates a signaling function.

In some embodiments, the disclosure provides a polynucleotide set that includes the following as part of one or more vectors:

(i) a first polynucleotide encoding a first fusion protein that includes a first dimerization polypeptide and a first signaling domain; and
(ii) a second polynucleotide encoding a second fusion protein that includes a second dimerization polypeptide, wherein interaction of the first and second dimerization polypeptides is mediated by a first small molecule regulator; and
(iii) a third polynucleotide encoding a third fusion protein that includes a third dimerization domain and a third signaling domain, wherein a second small molecule regulator disrupts binding of the first and second dimerization polypeptides and mediates binding of the third dimerization polypeptide to the first dimerization polypeptide thereby effectively regulates a second signaling function.

### Compositions Including Vectors

A polynucleotide set of the disclosure may be provided as part of a vector. In some embodiments, the first and second polynucleotide components of the polynucleotide set may be provided as part of a single vector.

The disclosure provides a composition that includes a single vector including:

(i) a first polynucleotide component encoding a first dimerization polypeptide, and
(ii) a second polynucleotide component encoding a second dimerization polypeptide, wherein either the first or second polynucleotide component further encodes a first signaling domain and the other of the first or second polynucleotide component further encodes a second signaling domain and interaction of the first and second dimerization polypeptides regulates a signaling function.

In some embodiments, the disclosure provides a composition that includes a single vector including:

(i) a first polynucleotide component encoding a first dimerization polypeptide, and
(ii) a second polynucleotide component encoding a second dimerization polypeptide, wherein either the first or second polynucleotide component further encodes an extracellular signaling domain and the other of the first or second polynucleotide component further encodes a cytoplasmic (intracellular) signaling domain and interaction of the first and second dimerization polypeptides regulates a signaling function.

In some aspects, the composition may be used for treating a subject in need of a therapy. The disclosure provides a pharmaceutical composition that includes:

(i) a single vector including a first polynucleotide component encoding a first dimerization domain and a second polynucleotide component encoding a second dimerization domain, wherein either the first or second polynucleotide component further encodes a first signaling domain and the other of the first or second polynucleotide component further encodes a second signaling domain, and
(ii) a pharmaceutically acceptable carrier, excipient, and/or stabilizer.

In some aspects, the disclosure provides a pharmaceutical composition that includes:

(i) a single vector including a first polynucleotide component encoding a first dimerization domain and a second polynucleotide component encoding a second dimerization domain, wherein either the first or second polynucleotide component further encodes an extracellular signaling domain and the other of the first or second polynucleotide component further encodes a cytoplasmic signaling domain, and
(ii) a pharmaceutically acceptable carrier, excipient, and/or stabilizer.

In some embodiments, the first and second polynucleotide components of the polynucleotide set may be provided as part of a set of at least two vectors, wherein, for example, a first vector includes the first polynucleotide component, and the second vector includes the second polynucleotide component.

The disclosure provides a composition that includes:

(i) a first vector including a first polynucleotide component encoding a first dimerization polypeptide, and
(ii) a second vector including a second polynucleotide component encoding a second dimerization polypeptide,
wherein either the first or second polynucleotide component further encodes a first signaling domain and the other of the first or second polynucleotide component further encodes a second signaling domain, and interaction of the first and second dimerization polypeptides regulates a signaling function.

In some aspects, the composition may be used for treating a subject in need of a therapy. The disclosure provides a pharmaceutical composition that includes:

(i) a first vector including a first polynucleotide component encoding a first dimerization polypeptide, and
(ii) a second vector including a second polynucleotide component encoding a second dimerization polypeptide, wherein either the first or second polynucleotide component further encodes a first signaling domain and the other of the first or second polynucleotide component further encodes a second signaling domain, and
(iii) a pharmaceutically acceptable carrier, excipient, and/or stabilizer.

In some aspects, the disclosure provides a pharmaceutical composition that includes:

(i) a first vector including a first polynucleotide component encoding a first dimerization domain, and
(ii) a second vector including a second polynucleotide component encoding a second dimerization domain, wherein either the first or second polynucleotide component further encodes an extracellular signaling domain and the other of the first or second polynucleotide component further encodes a cytoplasmic signaling domain, and
(iii) a pharmaceutically acceptable carrier, excipient, and/or stabilizer.

In some aspects, the disclosure provides a composition that includes:

(i) a first vector encoding a first fusion protein that includes a first dimerization polypeptide and a first signaling domain; and
(ii) a second vector encoding a second fusion protein that includes a second dimerization polypeptide, wherein interaction of the first and second dimerization polypeptides is mediated by a first small molecule regulator; and
(iii) a third vector encoding a third fusion protein that includes a third dimerization domain and a third signaling domain, wherein a second small molecule regulator disrupts binding of the first and second dimerization polypeptides and mediates binding of the third dimerization polypeptide to the first dimerization polypeptide.

In some aspects, the composition may be used for treating a subject in need of a therapy. The disclosure provides a pharmaceutical composition that includes:

(i) a first vector encoding a first fusion protein that includes a first dimerization polypeptide and a first signaling domain; and
(ii) a second vector encoding a second fusion protein that includes a second dimerization polypeptide, wherein interaction of the first and second dimerization polypeptides is mediated by a first small molecule regulator; and
(iii) a third vector encoding a third fusion protein that includes a third dimerization domain and a third signaling domain, wherein a second small molecule regulator disrupts binding of the first and second dimerization polypeptides and mediates binding of the third dimerization polypeptide to the first dimerization polypeptide, and
(iv) a pharmaceutically acceptable carrier, excipient, and/or stabilizer.

### Host Cells

The expression systems may be employed in cells, e.g., an in vitro, in vivo, or ex vivo cell comprising the nucleic acid disclosed herein or the expression systems disclosed herein.

In some aspects, the cell comprises a prokaryotic cell. In some aspects, the cell comprises a yeast cell. In some aspects, the cell comprises a mammalian cell. In some aspects, the mammalian cell comprises HEK293T, Jurkat, Jeko1, CHO, Cos, HeLa, HKB11, or BHK cells.

In some aspects, the cell (e.g., in vitro, in vivo, or ex vivo cells or any host cells) is a human cell. In some aspects, the cell is present in a patient or derived from a patient. In some aspects, the patient-derived cell is a tumor cell, cancer cell, immune cell, leukocyte, lymphocyte, T cell, regulatory T cell, effector T cell, CD4+ effector T cell, CD8+ effector T cell, memory T cell, autoreactive T cell, exhausted T cell, natural killer T cell (NKT cells), B cell, dendritic cell, macrophage, NK cell, innate lymphoid cell (ILC), cardiac cell, lung cell, muscle cell, epithelial cell, pancreatic cell, skin cell, CNS cell, neuron, myocyte, skeletal muscle cell, smooth muscle cell, liver cell, kidney cell, stem cell, induced pluripotent stem cell (iPSC), embryonic stem cell (ESC), and/or hematopoietic stem cell (HSC). In some aspects, the cell comprises an immune cell. In some aspects, the cell comprises a T cell. In some aspects, the cell comprises a regulatory T cell. In some aspects, the cell comprises a natural killer T cell. In some aspects, the cell comprises an NK cell. In some aspects, the cell comprises an effector T cell, e.g., a CD4+ effector T cell, and/or a CD8+ effector T cell.

In some aspects, the human cell is derived from an allogeneic donor. In some aspects, the allogeneic cell is a tumor cell, cancer cell, immune cell, leukocyte, lymphocyte, T cell, regulatory T cell, effector T cell, CD4+ effector T cell, CD8+ effector T cell, memory T cell, autoreactive T cell, exhausted T cell, natural killer T cell (NKT cells), B cell, dendritic cell, macrophage, NK cell, cardiac cell, lung cell, muscle cell, epithelial cell, pancreatic cell, skin cell, CNS cell, neuron, myocyte, skeletal muscle cell, smooth muscle cell, liver cell, kidney cell, induced pluripotent stem cell (iPSC), embryonic stem cell (ESC), and/or hematopoietic stem cell (HSC).

In some aspects, the human cell is derived from an autologous donor. In some aspects, the autologous cell is a tumor cell, cancer cell, immune cell, leukocyte, lymphocyte, T cell, regulatory T cell, effector T cell, CD4+ effector T cell, CD8+ effector T cell, memory T cell, autoreactive T cell, exhausted T cell, natural killer T cell (NKT cells), B cell, dendritic cell, macrophage, NK cell, cardiac cell, lung cell, muscle cell, epithelial cell, pancreatic cell, skin cell, CNS cell, neuron, myocyte, skeletal muscle cell, smooth muscle cell, liver cell, kidney cell, induced pluripotent stem cell (iPSC), embryonic stem cell (ESC), and/or hematopoietic stem cell (HSC).

In some aspects, the cells are engineered to comprise one or more nucleic acids encoding the polynucleotides described herein or to express the polypeptides described herein. In some aspects, the disclosure provides a host cell comprising the nucleic acid disclosed herein or the expression vector disclosed herein. In some aspects, the nucleic acid or the expression vector is integrated into a host cell chromosome. In some aspects, the nucleic acid or the expression vector is episomal.

### Compositions Including Host Cells

The polynucleotide set of the disclosure may be provided in a host cell. The cells can be transiently or stably engineered to incorporate the polynucleotide set of the disclosure. The disclosure provides a cell comprising a polynucleotide set that includes a first polynucleotide encoding a first fusion protein that includes a first signaling domain linked to a first dimerization polypeptide, and a second polynucleotide encoding a second fusion protein that includes a second signaling domain linked to a second dimerization polypeptide, wherein interaction of the first and second dimerization polypeptides controls a signaling function.

The disclosure provides a composition comprising a cell modified to express a polynucleotide set. In some aspects, the cell composition may be used for producing a polypeptide product of interest. The expressed polypeptide can be recovered from the cell free extract or recovered from the culture medium.

In some aspects, the composition may be used for treating a subject in need of a therapy. The disclosure provides a pharmaceutical composition that includes: (i) a cell which has been modified to express a polynucleotide set, and (ii) a pharmaceutically acceptable carrier, excipient, or stabilizer.

The cells may include polynucleotides of the disclosure expressing a gene of interest that provides a therapeutic benefit. Expression of the gene of interest may confer the cells with ability to attack tumor cells. The gene of interest may be a chimeric antigen receptor (CAR), e.g., a chimeric antigen receptor that targets tumor cells. The gene of interest may express a single-chain antibody fragment linked to a hinge linked to a transmembrane region. The transmembrane region may be linked to an intracellular signaling domain. The transmembrane region may be linked to a costimulatory domain.

The cells of the composition may, for example, be T cells. The cells of the composition may, for example, be CAR-T cells.

In some embodiments, the disclosure provides a cell composition comprising a means for reducing, ameliorating, or inhibiting exhaustion and/or dysfunction in a population of immune cells, e.g., immune cells expressing a CAR. In some aspects, the means comprise expressing the CAR as a gene of interest in a polynucleotide set.

### Methods of Making Small Molecules

The small molecules of the disclosure may be synthesized using known techniques. Danoprevir ((2R,6S,12Z,13aS,14aR,16aS)-14a-[(Cyclopropylsulfonyl)carbamoyl]-6-({[(2-methyl-2-propanyl)oxy]carbonyl}amino)-5,16-dioxo-1,2,3,5,6,7,8,9,10,11,13a,14,14a,15,16,16a-hexadecahydrocyclopropa[e]pyrrolo[1,2-a][1,4]diazacyclopentadecin-2-yl 4-fluoro-1,3-dihydro-2H-isoindole-2-carboxylate) may be synthesized using known techniques. See for example, Carreira, Erick Moran, Hisashi Yamamoto, and N. K. Yee. “Industrial Applications of Asymmetric Synthesis.” In Comprehensive Chirality 9, Amsterdam: Elsevier, 2012. Section 9.19.6, Danoprevir, the disclosure of which is incorporated herein by reference.

### Making Polynucleotides

The disclosure provides methods of producing the polynucleotides of the disclosure, such as DNA vectors of the disclosure and their subcomponents, as well as packaging vectors and plasmids of the disclosure. Standard molecular biology techniques may be used to assemble the polynucleotides of the disclosure. Polynucleotides can be chemically synthesized.

### Making Packaged Viral Capsids

The disclosure includes methods of making viral capsids containing polynucleotides of the disclosure. In general, viral capsids of the disclosure may be produced by supplying cells with packaging polynucleotides of the disclosure. The packaging polynucleotides may be supplied to packaging cells as plasmids. The packaging cells may be cultured to produce the viral capsids containing polynucleotides of the disclosure. Preferably the packaged viral capsids are replication incompetent.

A variety of commercially available kits are suitable for producing packaged viral capsids of the disclosure. Examples include: MISSION® Lentiviral Packaging Mix (available from Millipore Sigma); LV-Max Lentiviral Packaging Mix (available from ThermoFisher Scientific).

Viral capsid produced by packaging cells may be purified for use in downstream methods, such as delivery to cells for use in production of polypeptides, delivery to cells for use in cell-based therapies, or delivery to subjects for gene therapy methods. Purification may include processing to eliminate contaminants from host cells or culture media. Purification steps may include steps based on physical and/or chemical characteristics of the plasmids. Chemical characteristics may include, for example, hydrophilicity-hydrophobicity. Physical characteristics may include, for example, size. Examples of purification strategies based on particle size include density-gradient ultracentrifugation, ultrafiltration, precipitation, two-phase extraction systems and size exclusion chromatography. In some cases, precipitation may be employed together with centrifugation, e.g., using polyethylene glycol, ammonium sulfate or calcium phosphate. In some cases, aqueous two-phase separation systems with PEG, dextran or polyvinyl alcohol may be used. In some cases, membrane-based tangential flow filtration techniques are used; examples include ultrafiltration, diafiltration and microfiltration. In other embodiments, chromatographic means may be used for purifying viral capsids. In still other embodiments, immunoaffinity methods may be used to capture capsids using monoclonal antibodies having specificity to the relevant capsids. See Morenweiser, R., “Downstream processing of viral vectors and vaccines,” Gene Therapy (2005) 12, S103-S110 (2005), the entire disclosure of which is incorporated herein by reference.

Examples of suitable viral capsids include, but are not limited to, adenovirus, retrovirus, Lentivirus, Sendai virus vector, a baculovirus, Epstein Barr virus, a papovavirus, a vaccinia virus, a herpes simplex virus, and an adeno-associated virus (AAV).

### 1.1.2. Making Cells

The disclosure provides methods of making a modified cell to express a polypeptide product of interest.

In certain embodiments, the disclosure provides a method of making a therapeutic cell that expresses a polynucleotide set for use in treating a subject in need of a cell therapy. In one aspect, the disclosure provides a method of generating or preparing a therapeutic cell that expresses a polypeptide product of interest from a polynucleotide set integrated into a single vector. In one aspect, the disclosure provides a method of generating or preparing a therapeutic cell that expresses a polypeptide product of interest from a polynucleotide set integrated into two (or more) vectors.

In certain embodiments, the polynucleotides of the disclosure are maintained as extrachromosomal polynucleotides in the host cell. In certain embodiments, the polynucleotides of the disclosure are present in a vector (e.g., expression vector) in the host cell. In certain embodiments, the polynucleotides of the disclosure or a subset or subcomponents thereof, are integrated into a chromosome of the host cell.

Various methods can be used to introduce the expression vector encoding polynucleotides of the disclosure into cells to produce cells of the disclosure. See for example, Green, et al., Molecular cloning: A laboratory manual. Cold Spring Harbor, NY: Cold Spring Harbor Laboratory Press (2014).

Methods of introducing nucleic acid alterations to a gene of interest are well known in the art. Examples include targeted homologous recombination (e.g. “Hit and run”, “double-replacement”), site specific recombinases (e.g. the Cre recombinase and the Flp recombinase), PB transposases (e.g. Sleeping Beauty, piggyBac, Tol2 or Frog Prince), genome editing by engineered nucleases (e.g. meganucleases, Zinc finger nucleases (ZFNs), transcription-activator like effector nucleases (TALENs) and CRISPR/Cas system) and genome editing using recombinant adeno-associated virus (rAAV) platform. Agents for introducing nucleic acid alterations to a gene of interest can be designed using publicly available sources or obtained commercially from Transposagen, Addgene and Sangamo Biosciences. Vectors of the disclosure may make use of these methods for integrating polynucleotides of the disclosure into a host genome. Polynucleotides and vectors of the disclosure may include polynucleotides encoding polypeptides required for implementation of these methods for integrating polynucleotides of the disclosure into a host genome.

Various approaches suitable for integrating a polynucleotide(s) into a host cell genome are known in the art, including random integration or site-specific integration (e.g., a“landing pad” approach); see, e.g., Zhao, M. el al. (2018) Appl. Microbiol. Biotechnol. 102:6105-6117; Lee, J. S. et al. (2015) Sci. Rep. 5:8572; and Gaidukov, L. et al. (2018) Nucleic Acids Res. 46:4072-4086. Vectors of the disclosure may make use of these methods for integrating polynucleotides of the disclosure into a host genome. Vectors of the disclosure may include polynucleotides encoding polypeptides required for implementation of these methods for integrating polynucleotides of the disclosure into a host genome.

Host cells may be cultured using methods and compositions known in the art. Examples of commercially available media suitable for culturing host cells of the disclosure include Ham's F10 (Sigma), Minimal Essential Medium ((MEM), (Sigma), RP MI-1640 (Sigma), and Dulbecco's Modified Eagle's Medium ((DMEM), Sigma).

Culture media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. Culture conditions, such as temperature, pH, and the like, will be apparent to the ordinarily skilled artisan.

### 1.1.3. Making Polypeptides and Cellular Products

The disclosure provides methods of producing polypeptides. The methods may make use of cells of the disclosure treated with the small molecules of the disclosure.

The disclosure provides methods of producing a vector comprising a polynucleotide set, delivering the vector into a cell (e.g., in vivo, in vitro, or ex vivo), and expressing the polynucleotide set to provide and/or control a cellular function. Expression may be modulated by a small molecule of the disclosure.

In one embodiment, the method comprises the steps of (a) modifying a cell using a polynucleotide set encoding a polypeptide product of interest to yield a producer cell line; (b) culturing the producer cell line under conditions conducive for expression of the polypeptide product, (c) modulating production of the polypeptide product by delivering to the cell line a small molecule of the disclosure; and (d) optionally, recovering the expressed polypeptide.

In one embodiment, the method comprises the steps of (a) modifying a cell using a polynucleotide set encoding a polypeptide product of interest to yield a producer cell line; (b) culturing the producer cell line under conditions conducive for expression of the polypeptide product, (c) measuring the polypeptide of interest; (d) modulating production of the polypeptide product by delivering to the cell line a small molecule of the disclosure; and (e) optionally, recovering the expressed polypeptide.

The expressed polypeptide may, for example, be recovered from a cell free extract or recovered from the culture medium.

In one example, the polypeptide product of interest is a therapeutic protein or peptide.

In another example, the polypeptide product of interest is a virus or viral capsid selected from the group consisting of adenovirus, lentivirus, baculovirus, Epstein Barr virus, papovavirus, vaccinia virus, herpes virus, herpes simplex virus, retrovirus, and adeno-associated virus (AAV).

Polypeptide products of interest may be produced intracellularly, or directly secreted into the medium. If the polypeptide is produced intracellularly, cells may be lysed. Particulate debris may be removed, for example, by centrifugation or ultrafiltration. Where the polypeptide is secreted into the medium, supernatants from such expression systems may optionally be concentrated, e.g., using a commercially available protein concentration filter, for example, an Ami con or Millipore Pellicon ultrafiltration unit. A protease inhibitor such as PMSF may be included in any of the foregoing steps to inhibit proteolysis and antibiotics may be included to prevent the growth of adventitious contaminants.

Polypeptides may be purified using, for example, hydroxylapatite chromatography, gel electrophoresis, dialysis, and affinity chromatography, fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), low pH hydrophobic interaction chromatography, chromatofocusing, SDS-PAGE, ammonium sulfate precipitation, fractionation on immunoaffmity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration.

Polypeptide products of interest may be purified to obtain preparations that are substantially homogeneous for further assays and uses. Polypeptide products of interest may be purified to obtain preparations that are sufficiently homogenous for pharmaceutical uses.

### Cell Therapy Methods

The disclosure provides methods of treating a subject in need of a cell therapy. The method comprises the steps of (a) administering to the subject an effective amount of a pharmaceutical composition comprising a therapeutic cell encoding a polypeptide product of interest; and (b) administering a therapeutically effective amount of a small molecule to the subject.

In certain embodiments, the method includes the steps of:

(i) administering to the subject an effective amount of a pharmaceutical composition comprising a therapeutic cell encoding a therapeutic polypeptide product of interest (e.g., a CAR) and a pharmaceutically accepted carrier;
(ii) administering a therapeutically effective amount of a small molecule regulator to the subject;
(iii) monitoring the level of the therapeutic polypeptide product; and
(iv) optionally, adjusting the dosage of the small molecule regulator to adjust the level of the therapeutic polypeptide product in the subject to a desired level.

In certain embodiments, the method includes the steps of:

(i) administering to the subject an effective amount of a pharmaceutical composition comprising a therapeutic cell encoding a therapeutic polypeptide product of interest (e.g., a CAR) and a pharmaceutically acceptable carrier;
(ii) administering a therapeutically effective amount of a small molecule regulator to the subject;
(iii) monitoring the level of the therapeutic polypeptide product; and
(iv) optionally, administering a therapeutically effective amount of a second small molecule regulator to terminate the cell therapy.

In one aspect, the disclosure provides a method for treating a cancer, e.g., a tumor, in a subject in need thereof. Examples of cancers that can be treated using a pharmaceutical composition disclosed herein include, but are not limited to, melanomas, lymphomas, sarcomas, and cancers of the colon, kidney, stomach, bladder, brain (e.g., gliomas, glioblastomas, astrocytomas, medulloblastomas), prostate, bladder, rectum, esophagus, pancreas, liver, lung, breast, uterus, cervix, ovary, blood (e.g., acute myeloid leukemia, acute lymphoid leukemia, chronic myeloid leukemia, chronic lymphocytic leukemia, Burkitt's lymphoma, EBV-induced B-cell lymphoma).

In some cases, cells used to treat subjects may be autologous cells. In some cases, cells used to treat subjects may be allogenic cells.

In one aspect, the disclosure provides a method of controlling a T cell-mediated immune response in a subject in need thereof.

In one aspect, the disclosure provides a method of stimulating a T cell-mediated immune response to a target cell population or tissue in a subject.

In one aspect, the disclosure provides a method of providing an anti-tumor immunity in a subject.

In one aspect, the disclosure provides a method of controlling a T cell-mediated signaling activity in a subject in need thereof.

In one aspect, the disclosure provides a method of pausing a T cell-mediated signaling activity in a subject in need thereof.

In one aspect, the disclosure provides a method of terminating a T cell-mediated therapy in a subject in need thereof.

### Gene Therapy Methods

The disclosure provides methods of treating a subject in need thereof by delivering a polynucleotide set of the disclosure to a subject. A polynucleotide set of the disclosure may be delivered into a cell of a subject. The method may include administering a pharmaceutically effective amount of the polynucleotide set to the subject. Administration may be via administration of viral particles including one or more polynucleotides of the disclosure. Administration may be via administration of a pharmaceutical composition including one or more polynucleotides of the disclosure.

In certain embodiments, the method includes the steps of:

(i) administering to the subject an effective amount of a pharmaceutical composition including a polynucleotide set encoding a therapeutic polypeptide product of interest (e.g., a CAR) and a pharmaceutically acceptable carrier;
(ii) administering a therapeutically effective amount of a small molecule regulator to the subject;
(iii) monitoring the level of the therapeutic polypeptide product; and
(iv) optionally, adjusting the dosage of the small molecule regulator to adjust the level of the therapeutic polypeptide product in the subject to a desired level.

The subject may be a mammalian subject. The subject may be a human subject.

The following are examples of disease for which the gene therapy aspects disclosed herein may be useful:

### Blood Cell Disorders

Sickle cell anemia (Hb SS) >1 in 5,000; among African-Americans 1 in 400
Sickle-cell disease (Hb S/C) >1 in 25,000
Hb S/Beta-Thalassemia (Hb S/Th) >1 in 50,000
Variant hemoglobinopathies (including Hb E)
Glucose-6-phosphate dehydrogenase deficiency (G6PD)

### Inborn Errors of Amino Acid Metabolism

Tyrosinemia I (TYR I)<1 in 100,000
Argininosuccinic aciduria (ASA)<1 in 100,000
Citrullinemia (CIT)<1 in 100,000
Phenylketonuria (PKU) >1 in 25,000
Maple syrup urine disease (MSUD)<1 in 100,000
Homocystinuria (HCY)<1 in 100,000
Tyrosinemia II
Argininemia
Benign hyperphenylalaninemia
Defects of biopterin cofactor biosynthesis
Defects of biopterin cofactor regeneration
Tyrosinemia III
Hypermethioninemia
Citrullinemia type II

### Inborn Errors of Organic Acid Metabolism

Glutaric acidemia type I (GA I) >1 in 75,000
Hydroxymethylglutaryl lyase deficiency (HMG)<1 in 100,000
Isovaleric acidemia (IVA)<1 in 100,000
3-Methylcrotonyl-CoA carboxylase deficiency (3MCC) >1 in 75,000
Methylmalonyl-CoA mutase deficiency (MUT) >1 in 75,000
Methylmalonic aciduria, cblA and cblB forms (MMA, Cbl A,B)<1 in 100,000
Beta-ketothiolase deficiency (BKT)<1 in 100,000
Propionic acidemia (PROP) >1 in 75,000
Multiple-CoA carboxylase deficiency (MCD)<1 in 100,000
Methylmalonic acidemia (Cbl C,D)
Malonic acidemia
2-Methyl 3-hydroxy butyric aciduria
Isobutyryl-CoA dehydrogenase deficiency
2-Methylbutyryl-CoA dehydrogenase deficiency
3-Methylglutaconyl-CoA hydratase deficiency
Glutaric acidemia type II
HHH syndrome (Hyperammonemia, hyperornithinemia, homocitrullinuria syndrome)
Beta-methyl crotonyl carboxylase deficiency
Adenosylcobalamin synthesis defects

### Inborn Errors of Fatty Acid Metabolism

Long-chain hydroxyacyl-CoA dehydrogenase deficiency (LCHAD) >1 in 75,000
Medium-chain acyl-CoA dehydrogenase deficiency (MCAD)>1 in 25,000
Very-long-chain acyl-CoA dehydrogenase deficiency (VLCAD) >1 in 75,000
Trifunctional protein deficiency (TFP)<1 in 100,000
Carnitine uptake defect (CUD)<1 in 100,000
Medium/short-chain L-3-hydroxy acyl-CoA dehydrogenase deficiency
Medium-chain ketoacyl-CoA thiolase deficiency
Dienoyl-CoA reductase deficiency
Glutaric acidemia type II
Carnitine palmityl transferase deficiency type 1
Carnitine palmityl transferase deficiency type 2
Short-chain acyl-CoA dehydrogenase deficiency (SCAD)
Carnitine/acylcarnitine Translocase Deficiency (Translocase)
Short-chain hydroxy Acyl-CoA dehydrogenase deficiency (SCHAD)
Long-chain acyl-CoA dehydrogenase deficiency (LCAD)
Multiple acyl-CoA dehydrogenase deficiency (MADD)

### Miscellaneous Multisystem Diseases

Cystic fibrosis (CF) >1 in 5,000
Congenital hypothyroidism (CH) >1 in 5,000
Biotinidase deficiency (BIOT) >1 in 75,000
Congenital adrenal hyperplasia (CAH) >1 in 25,000
Classical galactosemia (GALT) >1 in 50,000
Galactokinase deficiency
Galactose epimerase deficiency

### Others

Severe combined immune deficiency (SCID)—added in 2009
Critical congenital heart defects (Screened using pulse oximetry)—added in 2010
Pompe disease—added in 2013
Mucopolysaccharidosis type I—added in 2015
X-linked adrenoleukodystrophy—added in 2018

TABLE A
Sequences

>hIgKSP-(scFV-hinge)-CD28tm-4-1BB-FKBP-P2A-
Dap10-CD28tm-41BB-FRB(T2098L)-tagBFP
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELASGSASGSASMGVQVETISPGDGRTFPKRGQTCVVHYTGMLED
GKKFDSSRDRNKPFKFMLGKQEVIRGWEEGVAQMSVGQRAKLTISPDYAY
GATGHPGIIPPHATLVFDVELLKLEGSGATNFSLLKQAGDVEENPGPMIH
LGHILFLLLLPVAAAQTTPGERSSLPAFYPGTSGSCSGCGSLSLPMFWVL
VVVGGVLACYSLLVTVAFIIFWVKRGRKKLLYIFKQPFMRPVQTTQEEDG
CSCRFPEEEEGGCELGSASGSASGSASGSGSILWHEMWHEGLEEASRLYF
GERNVKGMFEVLEPLHAMMERGPQTLKETSFNQAYGRDLMEAQEWCRKYM
KSGNVKDLLQAWDLYYHVFRRISKGSASGSASAGVSKGEELIKENMHMKL
YMEGTVDNHHFKCTSEGEGKPYEGTQTMRIKVVEGGPLPFAFDILATSFL
YGSKTFINHTQGIPDFFKQSFPEGFTWERVTTYEDGGVLTATQDTSLQDG
CLIYNVKIRGVNFTSNGPVMQKKTLGWEAFTETLYPADGGLEGRNDMALK
LVGGSHLIANAKTTYRSKKPAKNLKMPGVYYVDYRLERIKEANNETYVEQ
HEVAVARYCDLPSKLGHKLN
SEQ ID NO: 1
>hgKSP-(scFV-hinge)-CD28tm-4-1BB-FKBP-P2A-
Dap10-CD28tm-41BB-FRB(T2098L)-CD3z-tagBFP
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELASGSASGSASMGVQVETISPGDGRTFPKRGQTCVVHYTGMLED
GKKFDSSRDRNKPFKFMLGKQEVIRGWEEGVAQMSVGQRAKLTISPDYAY
GATGHPGIIPPHATLVEDVELLKLEGSGATNFSLLKQAGDVEENPGPMIH
LGHILFLLLLPVAAAQTTPGERSSLPAFYPGTSGSCSGCGSLSLPMFWVL
VVVGGVLACYSLLVTVAFIIFWVKRGRKKLLYIFKQPFMRPVQTTQEEDG
CSCRFPEEEEGGCELGSASGSASGSASGSGSILWHEMWHEGLEEASRLYF
GERNVKGMFEVLEPLHAMMERGPQTLKETSFNQAYGRDLMEAQEWCRKYM
KSGNVKDLLQAWDLYYHVFRRISKGSASGSASAGRVKFSRSADAPAYQQG
QNQLYNELNLGRREEYDVLDKRRGRDPEMGGKPRRKNPQEGLYNELQKDK
MAEAYSEIGMKGERRRGKGHDGLYQGLSTATKDTYDALHMQALPPRVSKG
EELIKENMHMKLYMEGTVDNHHFKCTSEGEGKPYEGTQTMRIKVVEGGPL
PFAFDILATSFLYGSKTFINHTQGIPDFFKQSFPEGFTWERVTTYEDGGV
LTATQDTSLQDGCLIYNVKIRGVNFTSNGPVMQKKTLGWEAFTETLYPAD
GGLEGRNDMALKLVGGSHLIANAKTTYRSKKPAKNLKMPGVYYVDYRLER
IKEANNETYVEQHEVAVARYCDLPSKLGHKLN
SEQ ID NO: 2
>hIgKSP-(scFV-hinge)-CD28tm-4-1BB-NS3aH1-P2A-
Dap10-CD28tm-41BB-GNCR1-CD3z-tagBFP
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELASGSASGSASMKKKGSVVIVGRINLSGDTAYSQQTRGLEGCQE
TSQTGRDKNQVEGEVQVVSTATQSFLATSINGVLWTVYHGAGTRTIASPK
GPVTQMYTNVDKDLVGWQAPQGSRSLTPCTCGSSDLYLVTRHADVIPVRR
RGDSRGSLLSPRPISYLKGSAGGPLLCPAGHAVGIFRAAVSTRGVAKAVD
FIPVESLETTMRSPGSGATNFSLLKQAGDVEENPGPMIHLGHILFLLLLP
VAAAQTTPGERSSLPAFYPGTSGSCSGCGSLSLPMFWVLVVVGGVLACYS
LLVTVAFIIFWVKRGRKKLLYIFKQPFMRPVQTTQEEDGCSCRFPEEEEG
GCELGSASGSASGSASGSGSDIEKLCKKAEEEAKEAQEKADELRQRHPDS
QAAEDAEDLANLAVAAVLTACLLAQEHPNADIAKLCIKAASEAAEAASKA
AELAQRHPDSQAARDAIKLASQAARAVILAIMLAAENPNADIAKLCIKAA
SEAAEAASKAAELAQRHPDSQAARDAIKLASQAAEAVERAIWLAAENPNA
DIAKKCIKAASEAAEEASKAAEEAQRHPDSQKARDEIKEASQKAEEVKER
CKSGSASGSASAGRVKFSRSADAPAYQQGQNQLYNELNLGRREEYDVLDK
RRGRDPEMGGKPRRKNPQEGLYNELQKDKMAEAYSEIGMKGERRRGKGHD
GLYQGLSTATKDTYDALHMQALPPRVSKGEELIKENMHMKLYMEGTVDNH
HFKCTSEGEGKPYEGTQTMRIKVVEGGPLPFAFDILATSFLYGSKTFINH
TQGIPDFFKQSFPEGFTWERVTTYEDGGVLTATQDTSLQDGCLIYNVKIR
GVNFTSNGPVMQKKTLGWEAFTETLYPADGGLEGRNDMALKLVGGSHLIA
NAKTTYRSKKPAKNLKMPGVYYVDYRLERIKEANNETYVEQHEVAVARYC
DLPSKLGHKLN
SEQ ID NO: 3
>hIgKSP-(scFV-hinge)-CD28tm-4-1BB-NS3aH1-P2A-
Dap10-CD28tm-41BB-DNCR2-CD3z-tagBFP
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELASGSASGSASMKKKGSVVIVGRINLSGDTAYSQQTRGLEGCQE
TSQTGRDKNQVEGEVQVVSTATQSFLATSINGVLWTVYHGAGTRTIASPK
GPVTQMYTNVDKDLVGWQAPQGSRSLTPCTCGSSDLYLVTRHADVIPVRR
RGDSRGSLLSPRPISYLKGSAGGPLLCPAGHAVGIFRAAVSTRGVAKAVD
FIPVESLETTMRSPGSGATNFSLLKQAGDVEENPGPMIHLGHILFLLLLP
VAAAQTTPGERSSLPAFYPGTSGSCSGCGSLSLPMFWVLVVVGGVLACYS
LLVTVAFIIFWVKRGRKKLLYIFKQPFMRPVQTTQEEDGCSCRFPEEEEG
GCELGSASGSASGSASGSGSSSDEEEARELIERAKEAAERAQEAAERTGD
PRVRELARELKRLAQEAAEEVKRDPSSSDVNEALKLIVEAIEAAVDALEA
AERTGDPEVRELARELVRLAVEAAEEVQRNPSSSDVNEALHSIVYAIEAA
IFALEAAERTGDPEVRELARELVRLAVEAAEEVQRNPSSRNVEHALMRIV
LAIYLAEENLREAEESGDPEKREKARERVREAVERAEEVQRDPSGWLNHG
SASGSASAGRVKFSRSADAPAYQQGQNQLYNELNLGRREEYDVLDKRRGR
DPEMGGKPRRKNPQEGLYNELQKDKMAEAYSEIGMKGERRRGKGHDGLYQ
GLSTATKDTYDALHMQALPPRVSKGEELIKENMHMKLYMEGTVDNHHFKC
TSEGEGKPYEGTQTMRIKVVEGGPLPFAFDILATSFLYGSKTFINHTQGI
PDFFKQSFPEGFTWERVTTYEDGGVLTATQDTSLQDGCLIYNVKIRGVNF
TSNGPVMQKKTLGWEAFTETLYPADGGLEGRNDMALKLVGGSHLIANAKT
TYRSKKPAKNLKMPGVYYVDYRLERIKEANNETYVEQHEVAVARYCDLPS
KLGHKLN
SEQ ID NO: 4
>hlgKSP-(scFV-hinge)-CD28tm-4-1BB-NS3aH1-P2A-
Dap10-CD28tm-41BB-ANR-CD3z-tagBFP
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELASGSASGSASMKKKGSVVIVGRINLSGDTAYSQQTRGLEGCQE
TSQTGRDKNQVEGEVQVVSTATQSFLATSINGVLWTVYHGAGTRTIASPK
GPVTQMYTNVDKDLVGWQAPQGSRSLTPCTCGSSDLYLVTRHADVIPVRR
RGDSRGSLLSPRPISYLKGSAGGPLLCPAGHAVGIFRAAVSTRGVAKAVD
FIPVESLETTMRSPGSGATNFSLLKQAGDVEENPGPMIHLGHILFLLLLP
VAAAQTTPGERSSLPAFYPGTSGSCSGCGSLSLPMFWVLVVVGGVLACYS
LLVTVAFIIFWVKRGRKKLLYIFKQPFMRPVQTTQEEDGCSCRFPEEEEG
GCELGSASGSASGSASGSGSGELDELVYLLDGPGYDPIHSDVVTRGGSHL
FNFGSASGSASAGRVKFSRSADAPAYQQGQNQLYNELNLGRREEYDVLDK
RRGRDPEMGGKPRRKNPQEGLYNELQKDKMAEAYSEIGMKGERRRGKGHD
GLYQGLSTATKDTYDALHMQALPPRVSKGEELIKENMHMKLYMEGTVDNH
HFKCTSEGEGKPYEGTQTMRIKVVEGGPLPFAFDILATSFLYGSKTFINH
TQGIPDFFKQSFPEGFTWERVTTYEDGGVLTATQDTSLQDGCLIYNVKIR
GVNFTSNGPVMQKKTLGWEAFTETLYPADGGLEGRNDMALKLVGGSHLIA
NAKTTYRSKKPAKNLKMPGVYYVDYRLERIKEANNETYVEQHEVAVARYC
DLPSKLGHKLN
SEQ ID NO: 5
>hIgKSP-(scFV-hinge)-CD28tm-4-1BB-BclxL-P2A-
Dap10-CD28tm-41BB-BadBH3-CD3z-tagBFP
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELASGSASGSASMSQSNRELVVDFLSYKLSQKGYSWSQFSDVEEN
RTEAPEGTESEMETPSAINGNPSWHLADSPAVNGATGHSSSLDAREVIPM
AAVKQALREAGDEFELRYRRAFSDLTSQLHITPGTAYQSFEQVVNELFRD
GVNWGRIVAFFSFGGALCVESVDKEMQVLVSRIAAWMATYLNDHLEPWIQ
ENGGWDTFVELYGNNGSGATNFSLLKQAGDVEENPGPMIHLGHILFLLLL
PVAAAQTTPGERSSLPAFYPGTSGSCSGCGSLSLPMFWVLVVVGGVLACY
SLLVTVAFIIFWVKRGRKKLLYIFKQPFMRPVQTTQEEDGCSCRFPEEEE
GGCELGSASGSASGSASGSGSAPPNLWAAQRYGRELRRMSDEFEGSFKGS
ASGSASAGRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPRVSKGEELIKENMHMKLYMEGTVDNHHFKCT
SEGEGKPYEGTQTMRIKVVEGGPLPFAFDILATSFLYGSKTFINHTQGIP
DFFKQSFPEGFTWERVTTYEDGGVLTATQDTSLQDGCLIYNVKIRGVNFT
SNGPVMQKKTLGWEAFTETLYPADGGLEGRNDMALKLVGGSHLIANAKTT
YRSKKPAKNLKMPGVYYVDYRLERIKEANNETYVEQHEVAVARYCDLPSK
LGHKLN
SEQ ID NO: 6
>hgKSP-(scFV-hinge)-CD28tm-4-1BB-CD3z
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELRVKFSRSADAPAYQQGQNQLYNELNLGRREEYDVLDKRRGRDP
EMGGKPRRKNPQEGLYNELQKDKMAEAYSEIGMKGERRRGKGHDGLYQGL
STATKDTYDALHMQALPPR
SEQ ID NO: 7
>Dap10-CD28tm-41BB-BadBH3-CD3z-P2a-EGFRt
MIHLGHILFLLLLPVAAAQTTPGERSSLPAFYPGTSGSCSGCGSLSLPMF
WVLVVVGGVLACYSLLVTVAFIIFWVKRGRKKLLYIFKQPFMRPVQTTQE
EDGCSCRFPEEEEGGCELGSASGSASGSASGSGSAPPNLWAAQRYGRELR
RMSDEFEGSFKGSASGSASAGRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPRGSGATNFSLLKQAGDVE
ENPGPMLLLVTSLLLCELPHPAFLLIPRKVCNGIGIGEFKDSLSINATNI
KHFKNCTSISGDLHILPVAFRGDSFTHTPPLDPQELDILKTVKEITGFLL
IQAWPENRTDLHAFENLEIIRGRTKQHGQFSLAVVSLNITSLGLRSLKEI
SDGDVIISGNKNLCYANTINWKKLFGTSGQKTKIISNRGENSCKATGQVC
HALCSPEGCWGPEPRDCVSCRNVSRGRECVDKCNLLEGEPREFVENSECI
QCHPECLPQAMNITCTGRGPDNCIQCAHYIDGPHCVKTCPAGVMGENNTL
VWKYADAGHVCHLCHPNCTYGCTGPGLEGCPTNGPKIPSIATGMVGALLL
LLVVALGIGLFM
SEQ ID NO: 8
>Dap10-CD28tm-41BB-ANR-CD3z-P2a-EGFRt
MIHLGHILFLLLLPVAAAQTTPGERSSLPAFYPGTSGSCSGCGSLSLPMF
WVLVVVGGVLACYSLLVTVAFIIFWVKRGRKKLLYIFKQPFMRPVQTTQE
EDGCSCRFPEEEEGGCELGSASGSASGSASGSGSGELDELVYLLDGPGYD
PIHSDVVTRGGSHLFNFGSASGSASAGRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPRGSGATNFSLLK
QAGDVEENPGPMLLLVTSLLLCELPHPAFLLIPRKVCNGIGIGEFKDSLS
INATNIKHFKNCTSISGDLHILPVAFRGDSFTHTPPLDPQELDILKTVKE
ITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFM
SEQ ID NO: 9
>Dap10-CD28tm-41BB-DNCR2-CD3z-P2a-EGFRt
MIHLGHILFLLLLPVAAAQTTPGERSSLPAFYPGTSGSCSGCGSLSLPMF
WVLVVVGGVLACYSLLVTVAFIIFWVKRGRKKLLYIFKQPFMRPVQTTQE
EDGCSCRFPEEEEGGCELGSASGSASGSASGSGSSSDEEEARELIERAKE
AAERAQEAAERTGDPRVRELARELKRLAQEAAEEVKRDPSSSDVNEALKL
IVEAIEAAVDALEAAERTGDPEVRELARELVRLAVEAAEEVQRNPSSSDV
NEALHSIVYAIEAAIFALEAAERTGDPEVRELARELVRLAVEAAEEVQRN
PSSRNVEHALMRIVLAIYLAEENLREAEESGDPEKREKARERVREAVERA
EEVQRDPSGWLNHGSASGSASAGRVKFSRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPRGSGATNFSLLKQAGD
VEENPGPMLLLVTSLLLCELPHPAFLLIPRKVCNGIGIGEFKDSLSINAT
NIKHFKNCTSISGDLHILPVAFRGDSFTHTPPLDPQELDILKTVKEITGF
LLIQAWPENRTDLHAFENLEIIRGRTKQHGQFSLAVVSLNITSLGLRSLK
EISDGDVIISGNKNLCYANTINWKKLFGTSGQKTKIISNRGENSCKATGQ
VCHALCSPEGCWGPEPRDCVSCRNVSRGRECVDKCNLLEGEPREFVENSE
CIQCHPECLPQAMNITCTGRGPDNCIQCAHYIDGPHCVKTCPAGVMGENN
TLVWKYADAGHVCHLCHPNCTYGCTGPGLEGCPTNGPKIPSIATGMVGAL
LLLLVVALGIGLFM
SEQ ID NO: 10
>Dap10-CD28tm-41BB-GNCR1-CD3z-P2a-EGFRt
MIHLGHILFLLLLPVAAAQTTPGERSSLPAFYPGTSGSCSGCGSLSLPMF
WVLVVVGGVLACYSLLVTVAFIIFWVKRGRKKLLYIFKQPFMRPVQTTQE
EDGCSCRFPEEEEGGCELGSASGSASGSASGSGSDIEKLCKKAEEEAKEA
QEKADELRQRHPDSQAAEDAEDLANLAVAAVLTACLLAQEHPNADIAKLC
IKAASEAAEAASKAAELAQRHPDSQAARDAIKLASQAARAVILAIMLAAE
NPNADIAKLCIKAASEAAEAASKAAELAQRHPDSQAARDAIKLASQAAEA
VERAIWLAAENPNADIAKKCIKAASEAAEEASKAAEEAQRHPDSQKARDE
IKEASQKAEEVKERCKSGSASGSASAGRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPRGSGATNFSLLK
QAGDVEENPGPMLLLVTSLLLCELPHPAFLLIPRKVCNGIGIGEFKDSLS
INATNIKHFKNCTSISGDLHILPVAFRGDSFTHTPPLDPQELDILKTVKE
ITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFM
SEQ ID NO: 11
>hgKSP-(scFV-hinge)-CD28tm-4-1BB-CD3z-P2a-EGFRt
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELRVKFSRSADAPAYQQGQNQLYNELNLGRREEYDVLDKRRGRDP
EMGGKPRRKNPQEGLYNELQKDKMAEAYSEIGMKGERRRGKGHDGLYQGL
STATKDTYDALHMQALPPRSGATNFSLLKQAGDVEENPGPMLLLVTSLLL
CELPHPAFLLIPRKVCNGIGIGEFKDSLSINATNIKHFKNCTSISGDLHI
LPVAFRGDSFTHTPPLDPQELDILKTVKEITGFLLIQAWPENRTDLHAFE
NLEIIRGRTKQHGQFSLAVVSLNITSLGLRSLKEISDGDVIISGNKNLCY
ANTINWKKLFGTSGQKTKIISNRGENSCKATGQVCHALCSPEGCWGPEPR
DCVSCRNVSRGRECVDKCNLLEGEPREFVENSECIQCHPECLPQAMNITC
TGRGPDNCIQCAHYIDGPHCVKTCPAGVMGENNTLVWKYADAGHVCHLCH
PNCTYGCTGPGLEGCPTNGPKIPSIATGMVGALLLLLVVALGIGLFM
SEQ ID NO: 12
>hIgKSP-(scFV-hinge)-CD28tm-4-1BB-BclxL
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELASGSASGSASMSQSNRELVVDFLSYKLSQKGYSWSQFSDVEEN
RTEAPEGTESEMETPSAINGNPSWHLADSPAVNGATGHSSSLDAREVIPM
AAVKQALREAGDEFELRYRRAFSDLTSQLHITPGTAYQSFEQVVNELFRD
GVNWGRIVAFFSFGGALCVESVDKEMQVLVSRIAAWMATYLNDHLEPWIQ
ENGGWDTFVELYGNN
SEQ ID NO: 13
>hIgKSP-(scFV-hinge)-CD28tm-4-1BB-NS3aH1gs
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELASGSASGSASMKKKGSVVIVGRINLSGDTAYSQQTRGLEGCQE
TSQTGRDKNQVEGEVQVVSTATQSFLATSINGVLWTVYHGAGTRTIASPK
GPVTQMYTNVDKDLVGWQAPQGSRSLTPCTCGSSDLYLVTRHADVIPVRR
RGDSRGSLLSPRPISYLKGSAGGPLLCPAGHAVGIFRAAVSTRGVAKAVD
FIPVESLETTMRSP
SEQ ID NO: 14
>DNCR2-Casp9gsD330A-P2a-EGFP-NLS
MSSDEEEARELIERAKEAAERAQEAAERTGDPRVRELARELKRLAQEAAE
EVKRDPSSSDVNEALKLIVEAIEAAVDALEAAERTGDPEVRELARELVRL
AVEAAEEVQRNPSSSDVNEALHSIVYAIEAAIFALEAAERTGDPEVRELA
RELVRLAVEAAEEVQRNPSSRNVEHALMRIVLAIYLAEENLREAEESGDP
EKREKARERVREAVERAEEVQRDPSGWLNHSGSASGSGGFGDVGALESLR
GNADLAYILSMEPCGHCLIINNVNFCRESGLRTRTGSNIDCEKLRRRFSS
LHFMVEVKGDLTAKKMVLALLELAQQDHGALDCCVVVILSHGCQASHLQF
PGAVYGTDGCPVSVEKIVNIFNGTSCPSLGGKPKLFFIQACGGEQKDHGF
EVASTSPEDESPGSNPEPDATPFQEGLRTFDQLAAISSLPTPSDIFVSYS
TFPGFVSWRDPKSGSWYVETLDDIFEQWAHSEDLQSLLLRVANAVSVKGI
YKQMPGCFNFLRKKLFFKTSGSGATNFSLLKQAGDVEENPGPMVSKGEEL
FTGVVPILVELDGDVNGHKFSVSGEGEGDATYGKLTLKFICTTGKLPVPW
PTLVTTLTYGVQCFSRYPDHMKQHDFFKSAMPEGYVQERTIFFKDDGNYK
TRAEVKFEGDTLVNRIELKGIDFKEDGNILGHKLEYNYNSHNVYIMADKQ
KNGIKVNFKIRHNIEDGSVQLADHYQQNTPIGDGPVLLPDNHYLSTQSAL
SKDPNEKRDHMVLLEFVTAAGITLGMDELYK
SEQ ID NO: 15
>iCasp-9 (FKBP-F36V-casp-9)
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGVDGFGDVGALESLRGNADLAYILSMEPCGHCLII
NNVNFCRESGLRTRTGSNIDCEKLRRRFSSLHFMVEVKGDLTAKKMVLAL
LELAQQDHGALDCCVVVILSHGCQASHLQFPGAVYGTDGCPVSVEKIVNI
FNGTSCPSLGGKPKLFFIQACGGEQKDHGFEVASTSPEDESPGSNPEPDA
TPFQEGLRTFDQLDAISSLPTPSDIFVSYSTFPGFVSWRDPKSGSWYVET
LDDIFEQWAHSEDLQSLLLRVANAVSVKGIYKQMPGCFNFLRKKLFFKTS
SEQ ID NO: 16
>iCasp-9-D330A (FKBP-F36V-casp-9)
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGVDGFGDVGALESLRGNADLAYILSMEPCGHCLII
NNVNFCRESGLRTRTGSNIDCEKLRRRFSSLHFMVEVKGDLTAKKMVLAL
LELAQQDHGALDCCVVVILSHGCQASHLQFPGAVYGTDGCPVSVEKIVNI
FNGTSCPSLGGKPKLFFIQACGGEQKDHGFEVASTSPEDESPGSNPEPDA
TPFQEGLRTFDQLAAISSLPTPSDIFVSYSTFPGFVSWRDPKSGSWYVET
LDDIFEQWAHSEDLQSLLLRVANAVSVKGIYKQMPGCFNFLRKKLFFKT
SEQ ID NO: 17
>rapaCasp-9 (FRB-FKBP-casp-9)
MASRILWHEMWHEGLEEASRLYFGERNVKGMFEVLEPLHAMMERGPQTLK
ETSFNQAYGRDLMEAQEWCRKYMKSGNVKDLLQAWDLYYHVFRRISKLEY
SGGGSLEGVQVETISPGDGRTFPKRGQTCVVHYTGMLEDGKKFDSSRDRN
KPFKFMLGKQEVIRGWEEGVAQMSVGQRAKLTISPDYAYGATGHPGIIPP
HATLVFDVELLKLESGGGGSGGGGSGGGGSGVDGFGDVGALESLRGNADL
AYILSMEPCGHCLIINNVNFCRESGLRTRTGSNIDCEKLRRRFSSLHFMV
EVKGDLTAKKMVLALLELAQQDHGALDCCVVVILSHGCQASHLQFPGAVY
GTDGCPVSVEKIVNIFNGTSCPSLGGKPKLFFIQACGGEQKDHGFEVAST
SPEDESPGSNPEPDATPFQEGLRTFDQLDAISSLPTPSDIFVSYSTFPGF
VSWRDPKSGSWYVETLDDIFEQWAHSEDLQSLLLRVANAVSVKGIYKQMP
GCFNFLRKKLFFKTS
SEQ ID NO: 18
>NS3a-Casp9-T2a-DNCR2-Casp9
MKKKGSVVIVGRINLSGDTAYAQQTRGEEGCQETSQTGRDKNQVEGEVQI
VSTATQTFLATSINGVLWTVYHGAGTRTIASPKGPVTQMYTNVDKDLVGW
QAPQGSRSLTPCTCGSSDLYLVTRHADVIPVRRRGDSRGSLLSPRPISYL
KGSAGGPLLCPAGHAVGIFRAAVSTRGVAKAVDFIPVESLETTMRSPSGG
GSGVDGFGDVGALESLRGNADLAYILSMEPCGHCLIINNVNFCRESGLRT
RTGSNIDCEKLRRRFSSLHFMVEVKGDLTAKKMVLALLELAQQDHGALDC
CVVVILSHGCQASHLQFPGAVYGTDGCPVSVEKIVNIFNGTSCPSLGGKP
KLFFIQACGGEQKDHGFEVASTSPEDESPGSNPEPDATPFQEGLRTFDQL
DAISSLPTPSDIFVSYSTFPGFVSWRDPKSGSWYVETLDDIFEQWAHSED
LQSLLLRVANAVSVKGIYKQMPGCFNFLRKKLFFKTSGSGEGRGSLLTCG
DVEENPGPSSDEEEARELIERAKEAAERAQEAAERTGDPRVRELARELKR
LAQEAAEEVKRDPSSSDVNEALKLIVEAIEAAVDALEAAERTGDPEVREL
ARELVRLAVEAAEEVQRNPSSSDVNEALHSIVYAIEAAIFALEAAERTGD
PEVRELARELVRLAVEAAEEVQRNPSSRNVEHALMRIVLAIYLAEENLRE
AEESGDPEKREKARERVREAVERAEEVQRDPSGWLNHSGGGSGVDGFGDV
GALESLRGNADLAYILSMEPCGHCLIINNVNFCRESGLRTRTGSNIDCEK
LRRRFSSLHFMVEVKGDLTAKKMVLALLELAQQDHGALDCCVVVILSHGC
QASHLQFPGAVYGTDGCPVSVEKIVNIFNGTSCPSLGGKPKLFFIQACGG
EQKDHGFEVASTSPEDESPGSNPEPDATPFQEGLRTFDQLDAISSLPTPS
DIFVSYSTFPGFVSWRDPKSGSWYVETLDDIFEQWAHSEDLQSLLLRVAN
AVSVKGIYKQMPGCFNFLRKKLFFKTS
SEQ ID NO: 19
>NS3a-Casp9-T2a-GNCR1-Casp9
MKKKGSVVIVGRINLSGDTAYAQQTRGEEGCQETSQTGRDKNQVEGEVQI
VSTATQTFLATSINGVLWTVYHGAGTRTIASPKGPVTQMYTNVDKDLVGW
QAPQGSRSLTPCTCGSSDLYLVTRHADVIPVRRRGDSRGSLLSPRPISYL
KGSAGGPLLCPAGHAVGIFRAAVSTRGVAKAVDFIPVESLETTMRSPSGG
GSGVDGFGDVGALESLRGNADLAYILSMEPCGHCLIINNVNFCRESGLRT
RTGSNIDCEKLRRRFSSLHFMVEVKGDLTAKKMVLALLELAQQDHGALDC
CVVVILSHGCQASHLQFPGAVYGTDGCPVSVEKIVNIFNGTSCPSLGGKP
KLFFIQACGGEQKDHGFEVASTSPEDESPGSNPEPDATPFQEGLRTFDQL
DAISSLPTPSDIFVSYSTFPGFVSWRDPKSGSWYVETLDDIFEQWAHSED
LQSLLLRVANAVSVKGIYKQMPGCFNFLRKKLFFKTSGSGEGRGSLLTCG
DVEENPGPDIEKLCKKAEEEAKEAQEKADELRQRHPDSQAAEDAEDLANL
AVAAVLTACLLAQEHPNADIAKLCIKAASEAAEAASKAAELAQRHPDSQA
ARDAIKLASQAARAVILAIMLAAENPNADIAKLCIKAASEAAEAASKAAE
LAQRHPDSQAARDAIKLASQAAEAVERAIWLAAENPNADIAKKCIKAASE
AAEEASKAAEEAQRHPDSQKARDEIKEASQKAEEVKERCKSSGGGSGVDG
FGDVGALESLRGNADLAYILSMEPCGHCLIINNVNFCRESGLRTRTGSNI
DCEKLRRRFSSLHFMVEVKGDLTAKKMVLALLELAQQDHGALDCCVVVIL
SHGCQASHLQFPGAVYGTDGCPVSVEKIVNIFNGTSCPSLGGKPKLFFIQ
ACGGEQKDHGFEVASTSPEDESPGSNPEPDATPFQEGLRTFDQLDAISSL
PTPSDIFVSYSTFPGFVSWRDPKSGSWYVETLDDIFEQWAHSEDLQSLLL
RVANAVSVKGIYKQMPGCFNFLRKKLFFKTS
SEQ ID NO: 20
>NS3a-DNCR2-Casp9
MKKKGSVVIVGRINLSGDTAYAQQTRGEEGCQETSQTGRDKNQVEGEVQI
VSTATQTFLATSINGVLWTVYHGAGTRTIASPKGPVTQMYTNVDKDLVGW
QAPQGSRSLTPCTCGSSDLYLVTRHADVIPVRRRGDSRGSLLSPRPISYL
KGSAGGPLLCPAGHAVGIFRAAVSTRGVAKAVDFIPVESLETTMRSPSGG
GSSSDEEEARELIERAKEAAERAQEAAERTGDPRVRELARELKRLAQEAA
EEVKRDPSSSDVNEALKLIVEAIEAAVDALEAAERTGDPEVRELARELVR
LAVEAAEEVQRNPSSSDVNEALHSIVYAIEAAIFALEAAERTGDPEVREL
ARELVRLAVEAAEEVQRNPSSRNVEHALMRIVLAIYLAEENLREAEESGD
PEKREKARERVREAVERAEEVQRDPSGWLNHSGGGGSGGGGSGVDGFGDV
GALESLRGNADLAYILSMEPCGHCLIINNVNFCRESGLRTRTGSNIDCEK
LRRRFSSLHFMVEVKGDLTAKKMVLALLELAQQDHGALDCCVVVILSHGC
QASHLQFPGAVYGTDGCPVSVEKIVNIFNGTSCPSLGGKPKLFFIQACGG
EQKDHGFEVASTSPEDESPGSNPEPDATPFQEGLRTFDQLDAISSLPTPS
DIFVSYSTFPGFVSWRDPKSGSWYVETLDDIFEQWAHSEDLQSLLLRVAN
AVSVKGIYKQMPGCFNFLRKKLFFKTS
SEQ ID NO: 21
>NS3a-GNCR1-Casp9
MKKKGSVVIVGRINLSGDTAYAQQTRGEEGCQETSQTGRDKNQVEGEVQI
VSTATQTFLATSINGVLWTVYHGAGTRTIASPKGPVTQMYTNVDKDLVGW
QAPQGSRSLTPCTCGSSDLYLVTRHADVIPVRRRGDSRGSLLSPRPISYL
KGSAGGPLLCPAGHAVGIFRAAVSTRGVAKAVDFIPVESLETTMRSPSGG
GSDIEKLCKKAEEEAKEAQEKADELRQRHPDSQAAEDAEDLANLAVAAVL
TACLLAQEHPNADIAKLCIKAASEAAEAASKAAELAQRHPDSQAARDAIK
LASQAARAVILAIMLAAENPNADIAKLCIKAASEAAEAASKAAELAQRHP
DSQAARDAIKLASQAAEAVERAIWLAAENPNADIAKKCIKAASEAAEEAS
KAAEEAQRHPDSQKARDEIKEASQKAEEVKERCKSSGGGGSGGGGSGVDG
FGDVGALESLRGNADLAYILSMEPCGHCLIINNVNFCRESGLRTRTGSNI
DCEKLRRRFSSLHFMVEVKGDLTAKKMVLALLELAQQDHGALDCCVVVIL
SHGCQASHLQFPGAVYGTDGCPVSVEKIVNIFNGTSCPSLGGKPKLFFIQ
ACGGEQKDHGFEVASTSPEDESPGSNPEPDATPFQEGLRTFDQLDAISSL
PTPSDIFVSYSTFPGFVSWRDPKSGSWYVETLDDIFEQWAHSEDLQSLLL
RVANAVSVKGIYKQMPGCFNFLRKKLFFKTS
SEQ ID NO: 22
>DNCR2-8link-NS3a-Casp9
MSSDEEEARELIERAKEAAERAQEAAERTGDPRVRELARELKRLAQEAAE
EVKRDPSSSDVNEALKLIVEAIEAAVDALEAAERTGDPEVRELARELVRL
AVEAAEEVQRNPSSSDVNEALHSIVYAIEAAIFALEAAERTGDPEVRELA
RELVRLAVEAAEEVQRNPSSRNVEHALMRIVLAIYLAEENLREAEESGDP
EKREKARERVREAVERAEEVQRDPSGWLNHSGGSGSGSKKKGSVVIVGRI
NLSGDTAYAQQTRGEEGCQETSQTGRDKNQVEGEVQIVSTATQTFLATSI
NGVLWTVYHGAGTRTIASPKGPVTQMYTNVDKDLVGWQAPQGSRSLTPCT
CGSSDLYLVTRHADVIPVRRRGDSRGSLLSPRPISYLKGSAGGPLLCPAG
HAVGIFRAAVSTRGVAKAVDFIPVESLETTMRSPSGGGGSGGGGSGVDGF
GDVGALESLRGNADLAYILSMEPCGHCLIINNVNFCRESGLRTRTGSNID
CEKLRRRFSSLHFMVEVKGDLTAKKMVLALLELAQQDHGALDCCVVVILS
HGCQASHLQFPGAVYGTDGCPVSVEKIVNIFNGTSCPSLGGKPKLFFIQA
CGGEQKDHGFEVASTSPEDESPGSNPEPDATPFQEGLRTFDQLDAISSLP
TPSDIFVSYSTFPGFVSWRDPKSGSWYVETLDDIFEQWAHSEDLQSLLLR
VANAVSVKGIYKQMPGCFNFLRKKLFFKTS
SEQ ID NO: 23
>DNCR2-5link-NS3a-Casp9
MSSDEEEARELIERAKEAAERAQEAAERTGDPRVRELARELKRLAQEAAE
EVKRDPSSSDVNEALKLIVEAIEAAVDALEAAERTGDPEVRELARELVRL
AVEAAEEVQRNPSSSDVNEALHSIVYAIEAAIFALEAAERTGDPEVRELA
RELVRLAVEAAEEVQRNPSSRNVEHALMRIVLAIYLAEENLREAEESGDP
EKREKARERVREAVERAEEVQRDPSGWLNHSGGGSKKKGSVVIVGRINLS
GDTAYAQQTRGEEGCQETSQTGRDKNQVEGEVQIVSTATQTFLATSINGV
LWTVYHGAGTRTIASPKGPVTQMYTNVDKDLVGWQAPQGSRSLTPCTCGS
SDLYLVTRHADVIPVRRRGDSRGSLLSPRPISYLKGSAGGPLLCPAGHAV
GIFRAAVSTRGVAKAVDFIPVESLETTMRSPSGGGGSGGGGSGVDGFGDV
GALESLRGNADLAYILSMEPCGHCLIINNVNFCRESGLRTRTGSNIDCEK
LRRRFSSLHFMVEVKGDLTAKKMVLALLELAQQDHGALDCCVVVILSHGC
QASHLQFPGAVYGTDGCPVSVEKIVNIFNGTSCPSLGGKPKLFFIQACGG
EQKDHGFEVASTSPEDESPGSNPEPDATPFQEGLRTFDQLDAISSLPTPS
DIFVSYSTFPGFVSWRDPKSGSWYVETLDDIFEQWAHSEDLQSLLLRVAN
AVSVKGIYKQMPGCFNFLRKKLFFKTS
SEQ ID NO: 24
>DNCR2-3link-NS3a-Casp9
MSSDEEEARELIERAKEAAERAQEAAERTGDPRVRELARELKRLAQEAAE
EVKRDPSSSDVNEALKLIVEAIEAAVDALEAAERTGDPEVRELARELVRL
AVEAAEEVQRNPSSSDVNEALHSIVYAIEAAIFALEAAERTGDPEVRELA
RELVRLAVEAAEEVQRNPSSRNVEHALMRIVLAIYLAEENLREAEESGDP
EKREKARERVREAVERAEEVQRDPSGWLNHSGSKKKGSVVIVGRINLSGD
TAYAQQTRGEEGCQETSQTGRDKNQVEGEVQIVSTATQTFLATSINGVLW
TVYHGAGTRTIASPKGPVTQMYTNVDKDLVGWQAPQGSRSLTPCTCGSSD
LYLVTRHADVIPVRRRGDSRGSLLSPRPISYLKGSAGGPLLCPAGHAVGI
FRAAVSTRGVAKAVDFIPVESLETTMRSPSGGGGSGGGGSGVDGFGDVGA
LESLRGNADLAYILSMEPCGHCLIINNVNFCRESGLRTRTGSNIDCEKLR
RRFSSLHFMVEVKGDLTAKKMVLALLELAQQDHGALDCCVVVILSHGCQA
SHLQFPGAVYGTDGCPVSVEKIVNIFNGTSCPSLGGKPKLFFIQACGGEQ
KDHGFEVASTSPEDESPGSNPEPDATPFQEGLRTFDQLDAISSLPTPSDI
FVSYSTFPGFVSWRDPKSGSWYVETLDDIFEQWAHSEDLQSLLLRVANAV
SVKGIYKQMPGCFNFLRKKLFFKTS
SEQ ID NO: 25
>DNCR2-noLink-NS3a-Casp9
MSSDEEEARELIERAKEAAERAQEAAERTGDPRVRELARELKRLAQEAAE
EVKRDPSSSDVNEALKLIVEAIEAAVDALEAAERTGDPEVRELARELVRL
AVEAAEEVQRNPSSSDVNEALHSIVYAIEAAIFALEAAERTGDPEVRELA
RELVRLAVEAAEEVQRNPSSRNVEHALMRIVLAIYLAEENLREAEESGDP
EKREKARERVREAVERAEEVQRDPSGWLNHKKKGSVVIVGRINLSGDTAY
AQQTRGEEGCQETSQTGRDKNQVEGEVQIVSTATQTFLATSINGVLWTVY
HGAGTRTIASPKGPVTQMYTNVDKDLVGWQAPQGSRSLTPCTCGSSDLYL
VTRHADVIPVRRRGDSRGSLLSPRPISYLKGSAGGPLLCPAGHAVGIFRA
AVSTRGVAKAVDFIPVESLETTMRSPSGGGGSGGGGSGVDGFGDVGALES
LRGNADLAYILSMEPCGHCLIINNVNFCRESGLRTRTGSNIDCEKLRRRF
SSLHFMVEVKGDLTAKKMVLALLELAQQDHGALDCCVVVILSHGCQASHL
QFPGAVYGTDGCPVSVEKIVNIFNGTSCPSLGGKPKLFFIQACGGEQKDH
GFEVASTSPEDESPGSNPEPDATPFQEGLRTFDQLDAISSLPTPSDIFVS
YSTFPGFVSWRDPKSGSWYVETLDDIFEQWAHSEDLQSLLLRVANAVSVK
GIYKQMPGCFNFLRKKLFFKTS
SEQ ID NO: 26
>D1-noLink-NS3a-Casp9
MDDNEAEKLIREAEEAAKDAEEAAERTGDPEVRELARELVRLAKEAAEEV
QRNPSSSDVNEALHSIVYAIEAAIFALEAAERTGDPEVRELARELVRLAV
EAAEEVQRNPSSRNVEHALMRIVLAIYLAEENLREKKKGSVVIVGRINLS
GDTAYSQQTRGLEGCQETSQTGRDKNQVEGEVQVVSTATQSFLATSINGV
LWTVYHGAGTRTIASPKGPVTQMYTNVDKDLVGWQAPQGSRSLTPCTCGS
SDLYLVTRHADVIPVRRRGDSRGSLLSPRPISYLKGSAGGPLLCPAGHAV
GIFRAAVSTRGVAKAVDFIPVESLETTMRSPSGSGGSGSASGSGGFGDVG
ALESLRGNADLAYILSMEPCGHCLIINNVNFCRESGLRTRTGSNIDCEKL
RRRFSSLHFMVEVKGDLTAKKMVLALLELAQQDHGALDCCVVVILSHGCQ
ASHLQFPGAVYGTDGCPVSVEKIVNIFNGTSCPSLGGKPKLFFIQACGGE
QKDHGFEVASTSPEDESPGSNPEPDATPFQEGLRTFDQLAAISSLPTPSD
IFVSYSTFPGFVSWRDPKSGSWYVETLDDIFEQWAHSEDLQSLLLRVANA
VSVKGIYKQMPGCFNFLRKKLFFKTS
SEQ ID NO: 27
>D9-NS3a-Casp9
MELEEVLKDLEEALERLRNEELRRLIEEIAELLRRRDDHSIIYMIEALIF
VIEAVERTGDERIEKLLEELIKRIRNGDDDSAIMRIVLAVYLIEENLREK
KKGSVVIVGRINLSGDTAYSQQTRGLEGCQETSQTGRDKNQVEGEVQVVS
TATQSFLATSINGVLWTVYHGAGTRTIASPKGPVTQMYTNVDKDLVGWQA
PQGSRSLTPCTCGSSDLYLVTRHADVIPVRRRGDSRGSLLSPRPISYLKG
SAGGPLLCPAGHAVGIFRAAVSTRGVAKAVDFIPVESLETTMRSPSGSGG
SGSASGSGGFGDVGALESLRGNADLAYILSMEPCGHCLIINNVNFCRESG
LRTRTGSNIDCEKLRRRFSSLHFMVEVKGDLTAKKMVLALLELAQQDHGA
LDCCVVVILSHGCQASHLQFPGAVYGTDGCPVSVEKIVNIFNGTSCPSLG
GKPKLFFIQACGGEQKDHGFEVASTSPEDESPGSNPEPDATPFQEGLRTF
DQLAAISSLPTPSDIFVSYSTFPGFVSWRDPKSGSWYVETLDDIFEQWAH
SEDLQSLLLRVANAVSVKGIYKQMPGCFNFLRKKLFFKTS
SEQ ID NO: 28
>D20-NS3a-Casp9
MYAKEAAEFAREAAERTGDPEVRELARELERLARRGSSEHALMRIVLAIY
LAEENLREKKKGSVVIVGRINLSGDTAYSQQTRGLEGCQETSQTGRDKNQ
VEGEVQVVSTATQSFLATSINGVLWTVYHGAGTRTIASPKGPVTQMYTNV
DKDLVGWQAPQGSRSLTPCTCGSSDLYLVTRHADVIPVRRRGDSRGSLLS
PRPISYLKGSAGGPLLCPAGHAVGIFRAAVSTRGVAKAVDFIPVESLETT
MRSPSGSGGSGSASGSGGFGDVGALESLRGNADLAYILSMEPCGHCLIIN
NVNFCRESGLRTRTGSNIDCEKLRRRFSSLHFMVEVKGDLTAKKMVLALL
ELAQQDHGALDCCVVVILSHGCQASHLQFPGAVYGTDGCPVSVEKIVNIF
NGTSCPSLGGKPKLFFIQACGGEQKDHGFEVASTSPEDESPGSNPEPDAT
PFQEGLRTFDQLAAISSLPTPSDIFVSYSTFPGFVSWRDPKSGSWYVETL
DDIFEQWAHSEDLQSLLLRVANAVSVKGIYKQMPGCFNFLRKKLFFKTS
SEQ ID NO: 29
-GNCR1-8link-NS3a-Casp9
MDIEKLCKKAEEEAKEAQEKADELRQRHPDSQAAEDAEDLANLAVAAVLT
ACLLAQEHPNADIAKLCIKAASEAAEAASKAAELAQRHPDSQAARDAIKL
ASQAARAVILAIMLAAENPNADIAKLCIKAASEAAEAASKAAELAQRHPD
SQAARDAIKLASQAAEAVERAIWLAAENPNADIAKKCIKAASEAAEEASK
AAEEAQRHPDSQKARDEIKEASQKAEEVKERCKSSGGSGSGSKKKGSVVI
VGRINLSGDTAYAQQTRGEEGCQETSQTGRDKNQVEGEVQIVSTATQTFL
ATSINGVLWTVYHGAGTRTIASPKGPVTQMYTNVDKDLVGWQAPQGSRSL
TPCTCGSSDLYLVTRHADVIPVRRRGDSRGSLLSPRPISYLKGSAGGPLL
CPAGHAVGIFRAAVSTRGVAKAVDFIPVESLETTMRSPSGGGGSGGGGSG
VDGFGDVGALESLRGNADLAYILSMEPCGHCLIINNVNFCRESGLRTRTG
SNIDCEKLRRRFSSLHFMVEVKGDLTAKKMVLALLELAQQDHGALDCCVV
VILSHGCQASHLQFPGAVYGTDGCPVSVEKIVNIFNGTSCPSLGGKPKLF
FIQACGGEQKDHGFEVASTSPEDESPGSNPEPDATPFQEGLRTFDQLDAI
SSLPTPSDIFVSYSTFPGFVSWRDPKSGSWYVETLDDIFEQWAHSEDLQS
LLLRVANAVSVKGIYKQMPGCFNFLRKKLFFKTS
SEQ ID NO: 30
-GNCR1-5link-NS3a-Casp9
MDIEKLCKKAEEEAKEAQEKADELRQRHPDSQAAEDAEDLANLAVAAVLT
ACLLAQEHPNADIAKLCIKAASEAAEAASKAAELAQRHPDSQAARDAIKL
ASQAARAVILAIMLAAENPNADIAKLCIKAASEAAEAASKAAELAQRHPD
SQAARDAIKLASQAAEAVERAIWLAAENPNADIAKKCIKAASEAAEEASK
AAEEAQRHPDSQKARDEIKEASQKAEEVKERCKSSGGGSKKKGSVVIVGR
INLSGDTAYAQQTRGEEGCQETSQTGRDKNQVEGEVQIVSTATQTFLATS
INGVLWTVYHGAGTRTIASPKGPVTQMYTNVDKDLVGWQAPQGSRSLTPC
TCGSSDLYLVTRHADVIPVRRRGDSRGSLLSPRPISYLKGSAGGPLLCPA
GHAVGIFRAAVSTRGVAKAVDFIPVESLETTMRSPSGGGGSGGGGSGVDG
FGDVGALESLRGNADLAYILSMEPCGHCLIINNVNFCRESGLRTRTGSNI
DCEKLRRRFSSLHFMVEVKGDLTAKKMVLALLELAQQDHGALDCCVVVIL
SHGCQASHLQFPGAVYGTDGCPVSVEKIVNIFNGTSCPSLGGKPKLFFIQ
ACGGEQKDHGFEVASTSPEDESPGSNPEPDATPFQEGLRTFDQLDAISSL
PTPSDIFVSYSTFPGFVSWRDPKSGSWYVETLDDIFEQWAHSEDLQSLLL
RVANAVSVKGIYKQMPGCFNFLRKKLFFKTS
SEQ ID NO: 31
>GNCR1-3link-NS3a-Casp9
MDIEKLCKKAEEEAKEAQEKADELRQRHPDSQAAEDAEDLANLAVAAVLT
ACLLAQEHPNADIAKLCIKAASEAAEAASKAAELAQRHPDSQAARDAIKL
ASQAARAVILAIMLAAENPNADIAKLCIKAASEAAEAASKAAELAQRHPD
SQAARDAIKLASQAAEAVERAIWLAAENPNADIAKKCIKAASEAAEEASK
AAEEAQRHPDSQKARDEIKEASQKAEEVKERCKSSGSKKKGSVVIVGRIN
LSGDTAYAQQTRGEEGCQETSQTGRDKNQVEGEVQIVSTATQTFLATSIN
GVLWTVYHGAGTRTIASPKGPVTQMYTNVDKDLVGWQAPQGSRSLTPCTC
GSSDLYLVTRHADVIPVRRRGDSRGSLLSPRPISYLKGSAGGPLLCPAGH
AVGIFRAAVSTRGVAKAVDFIPVESLETTMRSPSGGGGSGGGGSGVDGFG
DVGALESLRGNADLAYILSMEPCGHCLIINNVNFCRESGLRTRTGSNIDC
EKLRRRFSSLHFMVEVKGDLTAKKMVLALLELAQQDHGALDCCVVVILSH
GCQASHLQFPGAVYGTDGCPVSVEKIVNIFNGTSCPSLGGKPKLFFIQAC
GGEQKDHGFEVASTSPEDESPGSNPEPDATPFQEGLRTFDQLDAISSLPT
PSDIFVSYSTFPGFVSWRDPKSGSWYVETLDDIFEQWAHSEDLQSLLLRV
ANAVSVKGIYKQMPGCFNFLRKKLFFKTS
SEQ ID NO: 32
>GNCR1-noLink-NS3a-Casp9
MDIEKLCKKAEEEAKEAQEKADELRQRHPDSQAAEDAEDLANLAVAAVLT
ACLLAQEHPNADIAKLCIKAASEAAEAASKAAELAQRHPDSQAARDAIKL
ASQAARAVILAIMLAAENPNADIAKLCIKAASEAAEAASKAAELAQRHPD
SQAARDAIKLASQAAEAVERAIWLAAENPNADIAKKCIKAASEAAEEASK
AAEEAQRHPDSQKARDEIKEASQKAEEVKERCKSKKKGSVVIVGRINLSG
DTAYAQQTRGEEGCQETSQTGRDKNQVEGEVQIVSTATQTFLATSINGVL
WTVYHGAGTRTIASPKGPVTQMYTNVDKDLVGWQAPQGSRSLTPCTCGSS
DLYLVTRHADVIPVRRRGDSRGSLLSPRPISYLKGSAGGPLLCPAGHAVG
IFRAAVSTRGVAKAVDFIPVESLETTMRSPSGGGGSGGGGSGVDGFGDVG
ALESLRGNADLAYILSMEPCGHCLIINNVNFCRESGLRTRTGSNIDCEKL
RRRFSSLHFMVEVKGDLTAKKMVLALLELAQQDHGALDCCVVVILSHGCQ
ASHLQFPGAVYGTDGCPVSVEKIVNIFNGTSCPSLGGKPKLFFIQACGGE
QKDHGFEVASTSPEDESPGSNPEPDATPFQEGLRTFDQLDAISSLPTPSD
IFVSYSTFPGFVSWRDPKSGSWYVETLDDIFEQWAHSEDLQSLLLRVANA
VSVKGIYKQMPGCFNFLRKKLFFKTS
SEQ ID NO: 33
>DNCR2-Casp9D330A
MSSDEEEARELIERAKEAAERAQEAAERTGDPRVRELARELKRLAQEAAE
EVKRDPSSSDVNEALKLIVEAIEAAVDALEAAERTGDPEVRELARELVRL
AVEAAEEVQRNPSSSDVNEALHSIVYAIEAAIFALEAAERTGDPEVRELA
RELVRLAVEAAEEVQRNPSSRNVEHALMRIVLAIYLAEENLREAEESGDP
EKREKARERVREAVERAEEVQRDPSGWLNHSGSASGSGGFGDVGALESLR
GNADLAYILSMEPCGHCLIINNVNFCRESGLRTRTGSNIDCEKLRRRFSS
LHFMVEVKGDLTAKKMVLALLELAQQDHGALDCCVVVILSHGCQASHLQF
PGAVYGTDGCPVSVEKIVNIFNGTSCPSLGGKPKLFFIQACGGEQKDHGF
EVASTSPEDESPGSNPEPDATPFQEGLRTFDQLAAISSLPTPSDIFVSYS
TFPGFVSWRDPKSGSWYVETLDDIFEQWAHSEDLQSLLLRVANAVSVKGI
YKQMPGCFNFLRKKLFFKTS
SEQ ID NO: 34
>DNCR2-Casp9
MSSDEEEARELIERAKEAAERAQEAAERTGDPRVRELARELKRLAQEAAE
EVKRDPSSSDVNEALKLIVEAIEAAVDALEAAERTGDPEVRELARELVRL
AVEAAEEVQRNPSSSDVNEALHSIVYAIEAAIFALEAAERTGDPEVRELA
RELVRLAVEAAEEVQRNPSSRNVEHALMRIVLAIYLAEENLREAEESGDP
EKREKARERVREAVERAEEVQRDPSGWLNHSGSASGSGGFGDVGALESLR
GNADLAYILSMEPCGHCLIINNVNFCRESGLRTRTGSNIDCEKLRRRFSS
LHFMVEVKGDLTAKKMVLALLELAQQDHGALDCCVVVILSHGCQASHLQF
PGAVYGTDGCPVSVEKIVNIFNGTSCPSLGGKPKLFFIQACGGEQKDHGF
EVASTSPEDESPGSNPEPDATPFQEGLRTFDQLDAISSLPTPSDIFVSYS
TFPGFVSWRDPKSGSWYVETLDDIFEQWAHSEDLQSLLLRVANAVSVKGI
YKQMPGCFNFLRKKLFFKTS
SEQ ID NO: 35
>D1-Casp9D330A
MDDNEAEKLIREAEEAAKDAEEAAERTGDPEVRELARELVRLAKEAAEEV
QRNPSSSDVNEALHSIVYAIEAAIFALEAAERTGDPEVRELARELVRLAV
EAAEEVQRNPSSRNVEHALMRIVLAIYLAEENLRESGSASGSGGFGDVGA
LESLRGNADLAYILSMEPCGHCLIINNVNFCRESGLRTRTGSNIDCEKLR
RRFSSLHFMVEVKGDLTAKKMVLALLELAQQDHGALDCCVVVILSHGCQA
SHLQFPGAVYGTDGCPVSVEKIVNIFNGTSCPSLGGKPKLFFIQACGGEQ
KDHGFEVASTSPEDESPGSNPEPDATPFQEGLRTFDQLAAISSLPTPSDI
FVSYSTFPGFVSWRDPKSGSWYVETLDDIFEQWAHSEDLQSLLLRVANAV
SVKGIYKQMPGCFNFLRKKLFFKTS
SEQ ID NO: 36
>D9-Casp9D330A
MELEEVLKDLEEALERLRNEELRRLIEEIAELLRRRDDHSIIYMIEALIF
VIEAVERTGDERIEKLLEELIKRIRNGDDDSAIMRIVLAVYLIEENLRES
GSASGSGGFGDVGALESLRGNADLAYILSMEPCGHCLIINNVNFCRESGL
RTRTGSNIDCEKLRRRFSSLHFMVEVKGDLTAKKMVLALLELAQQDHGAL
DCCVVVILSHGCQASHLQFPGAVYGTDGCPVSVEKIVNIFNGTSCPSLGG
KPKLFFIQACGGEQKDHGFEVASTSPEDESPGSNPEPDATPFQEGLRTFD
QLAAISSLPTPSDIFVSYSTFPGFVSWRDPKSGSWYVETLDDIFEQWAHS
EDLQSLLLRVANAVSVKGIYKQMPGCFNFLRKKLFFKTS
SEQ ID NO: 37
>D20-Casp9D330A
MYAKEAAEFAREAAERTGDPEVRELARELERLARRGSSEHALMRIVLAIY
LAEENLRESGSASGSGGFGDVGALESLRGNADLAYILSMEPCGHCLIINN
VNFCRESGLRTRTGSNIDCEKLRRRFSSLHFMVEVKGDLTAKKMVLALLE
LAQQDHGALDCCVVVILSHGCQASHLQFPGAVYGTDGCPVSVEKIVNIFN
GTSCPSLGGKPKLFFIQACGGEQKDHGFEVASTSPEDESPGSNPEPDATP
FQEGLRTFDQLAAISSLPTPSDIFVSYSTFPGFVSWRDPKSGSWYVETLD
DIFEQWAHSEDLQSLLLRVANAVSVKGIYKQMPGCFNFLRKKLFFKTS
SEQ ID NO: 38
>GNCR1-Casp9D330A
MDIEKLCKKAEEEAKEAQEKADELRQRHPDSQAAEDAEDLANLAVAAVLT
ACLLAQEHPNADIAKLCIKAASEAAEAASKAAELAQRHPDSQAARDAIKL
ASQAARAVILAIMLAAENPNADIAKLCIKAASEAAEAASKAAELAQRHPD
SQAARDAIKLASQAAEAVERAIWLAAENPNADIAKKCIKAASEAAEEASK
AAEEAQRHPDSQKARDEIKEASQKAEEVKERCKSGSASGSGGFGDVGALE
SLRGNADLAYILSMEPCGHCLIINNVNFCRESGLRTRTGSNIDCEKLRRR
FSSLHFMVEVKGDLTAKKMVLALLELAQQDHGALDCCVVVILSHGCQASH
LQFPGAVYGTDGCPVSVEKIVNIFNGTSCPSLGGKPKLFFIQACGGEQKD
HGFEVASTSPEDESPGSNPEPDATPFQEGLRTFDQLAAISSLPTPSDIFV
SYSTFPGFVSWRDPKSGSWYVETLDDIFEQWAHSEDLQSLLLRVANAVSV
KGIYKQMPGCFNFLRKKLFFKTS
SEQ ID NO: 39
>GNCR1-Casp9
MDIEKLCKKAEEEAKEAQEKADELRQRHPDSQAAEDAEDLANLAVAAVLT
ACLLAQEHPNADIAKLCIKAASEAAEAASKAAELAQRHPDSQAARDAIKL
ASQAARAVILAIMLAAENPNADIAKLCIKAASEAAEAASKAAELAQRHPD
SQAARDAIKLASQAAEAVERAIWLAAENPNADIAKKCIKAASEAAEEASK
AAEEAQRHPDSQKARDEIKEASQKAEEVKERCKSGSASGSGGFGDVGALE
SLRGNADLAYILSMEPCGHCLIINNVNFCRESGLRTRTGSNIDCEKLRRR
FSSLHFMVEVKGDLTAKKMVLALLELAQQDHGALDCCVVVILSHGCQASH
LQFPGAVYGTDGCPVSVEKIVNIFNGTSCPSLGGKPKLFFIQACGGEQKD
HGFEVASTSPEDESPGSNPEPDATPFQEGLRTFDQLDAISSLPTPSDIFV
SYSTFPGFVSWRDPKSGSWYVETLDDIFEQWAHSEDLQSLLLRVANAVSV
KGIYKQMPGCFNFLRKKLFFKTS
SEQ ID NO: 40
>hgKSP-(scFV-hinge)-CD28tm-4-1BB-NS3aH1
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELASGSASGSASMKKKGSVVIVGRINLSGDTAYSQQTRGLEGCQE
TSQTGRDKNQVEGEVQVVSTATQSFLATSINGVLWTVYHGAGTRTIASPK
GPVTQMYTNVDKDLVGWQAPQGSRSLTPCTCGSSDLYLVTRHADVIPVRR
RGDSRGSLLSPRPISYLKGSAGGPLLCPAGHAVGIFRAAVSTRGVAKAVD
FIPVESLETTMRSP
SEQ ID NO: 41
>dimer-NS3aH1
MTRTEIIRELERSLREQEELAKRLKELLRELERLQREGSSDEDVRELLRE
IKELVEEIEKLAREQKYLVEELKRQDGSASGSASMKKKGSVVIVGRINLS
GDTAYSQQTRGLEGCQETSQTGRDKNQVEGEVQVVSTATQSFLATSINGV
LWTVYHGAGTRTIASPKGPVTQMYTNVDKDLVGWQAPQGSRSLTPCTCGS
SDLYLVTRHADVIPVRRRGDSRGSLLSPRPISYLKGSAGGPLLCPAGHAV
GIFRAAVSTRGVAKAVDFIPVESLETTMRSP
SEQ ID NO: 42
>hexamer-NS3a
MTEDEIRKLRKLLEEAEKKLKKLEDKTRRSEEISKTDDDPKAQSLQLIAE
SLMLIAESLLIIAISLLLSSRNGSASGSASMKKKGSVVIVGRINLSGDTA
YSQQTRGLEGCQETSQTGRDKNQVEGEVQVVSTATQSFLATSINGVLWTV
YHGAGTRTIASPKGPVTQMYTNVDKDLVGWQAPQGSRSLTPCTCGSSDLY
LVTRHADVIPVRRRGDSRGSLLSPRPISYLKGSAGGPLLCPAGHAVGIFR
AAVSTRGVAKAVDFIPVESLETTMRSP
SEQ ID NO: 43
>pentamer-NS3aH1
MTRRKQEMKRLKKEMEKIREETEEVKKEIEESKKRPQSESAKNLILIMQL
LINQIRLLALQIRMLALQLQEGSASGSASMKKKGSVVIVGRINLSGDTAY
SQQTRGLEGCQETSQTGRDKNQVEGEVQVVSTATQSFLATSINGVLWTVY
HGAGTRTIASPKGPVTQMYTNVDKDLVGWQAPQGSRSLTPCTCGSSDLYL
VTRHADVIPVRRRGDSRGSLLSPRPISYLKGSAGGPLLCPAGHAVGIFRA
AVSTRGVAKAVDFIPVESLETTMRSP
SEQ ID NO: 44
>trimer-NS3aH1
MSEYEIRKALEELKASTAELKRATASLRASTEELKKNPSEDALVENNRLI
VEHNAIIVENNRIIAAVLELIVRAIKGSASGSASMKKKGSVVIVGRINLS
GDTAYSQQTRGLEGCQETSQTGRDKNQVEGEVQVVSTATQSFLATSINGV
LWTVYHGAGTRTIASPKGPVTQMYTNVDKDLVGWQAPQGSRSLTPCTCGS
SDLYLVTRHADVIPVRRRGDSRGSLLSPRPISYLKGSAGGPLLCPAGHAV
GIFRAAVSTRGVAKAVDFIPVESLETTMRSP
SEQ ID NO: 45
>hIgKSP-(scFV-hinge)-CD28tm-4-1BB-NS3aH1-NS3aH1
MVLQTQVFISLLLWISGAYG(scFV_hinge)CPPCPMFWVLVVVGGVLA
CYSLLVTVAFIIFWVKRGRKKLLYIFKQPFMRPVQTTQEEDGCSCRFPEE
EEGGCELASGSASGSASMKKKGSVVIVGRINLSGDTAYSQQTRGLEGCQE
TSQTGRDKNQVEGEVQVVSTATQSFLATSINGVLWTVYHGAGTRTIASPK
GPVTQMYTNVDKDLVGWQAPQGSRSLTPCTCGSSDLYLVTRHADVIPVRR
RGDSRGSLLSPRPISYLKGSAGGPLLCPAGHAVGIFRAAVSTRGVAKAVD
FIPVESLETTMRSPGSSGSSGSSGGKKKGSVVIVGRINLSGDTAYSQQTR
GLEGCQETSQTGRDKNQVEGEVQVVSTATQSFLATSINGVLWTVYHGAGT
RTIASPKGPVTQMYTNVDKDLVGWQAPQGSRSLTPCTCGSSDLYLVTRHA
DVIPVRRRGDSRGSLLSPRPISYLKGSAGGPLLCPAGHAVGIFRAAVSTR
GVAKAVDFIPVESLETTMRSP
SEQ ID NO: 46
>FKBP(F36V)-Casp-1
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGSGNPAMPTSSGSEGNVKLCSLEEAQRIWKQKSAE
IYPIMDKSSRTRLALIICNEEFDSIPRRTGAEVDITGMTMLLQNLGYSVD
VKKNLTASDMTTELEAFAHRPEHKTSDSTFLVFMSHGIREGICGKKHSEQ
VPDILQLNAIFNMLNTKNCPSLKDKPKVIIIQACRGDSPGVVWFKDSVGV
SGNLSLPTTEEFEDDAIKKAHIEKDFIAFCSSTPDNVSWRHPTMGSVFIG
RLIEHMQEYACSCDVEEIFRKVRFSFEQPDGRAQMPTTERVTLTRCFYLF
PGH
SEQ ID NO: 47
>FKBP(F36V)-Casp-4
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGSGEAGPPESGESTDALKLCPHEEFLRLCKERAEE
IYPIKERNNRTRLALIICNTEFDHLPPRNGADFDITGMKELLEGLDYSVD
VEENLTARDMESALRAFATRPEHKSSDSTFLVLMSHGILEGICGTVHDEK
KPDVLLYDTIFQIFNNRNCLSLKDKPKVIIVQACRGANRGELWVRDSPAS
LEVASSQSSENLEEDAVYKTHVEKDFIAFCSSTPHNVSWRDSTMGSIFIT
QLITCFQKYSWCCHLEEVFRKVQQSFETPRAKAQMPTIERLSMTRYFYLF
PGN
SEQ ID NO: 48
>FKBP(F36V)-Casp-3
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGMENTENSVDSKSIKNLEPKIIHGSESMDSGISLD
NSYKMDYPEMGLCIIINNKNFHKSTGMTSRSGTDVDAANLRETFRNLKYE
VRNKNDLTREEIVELMRDVSKEDHSKRSSFVCVLLSHGEEGIIFGTNGPV
DLKKITNFFRGDRCRSLTGKPKLFIIQACRGTELDCGIETDSGVDDDMAC
HKIPVEADFLYAYSTAPGYYSWRNSKDGSWFIQSLCAMLKQYADKLEFMH
ILTRVNRKVATEFESFSFDATFHAKKQIPCIVSMLTKELYFYH
SEQ ID NO: 49
>FKBP(F36V)-Casp-3-Casp-9interface
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGMENTENSVDSKSIKNLEPKIIHGSESMDSGISLD
NSYKMDYPEMGLCIIINNKNFHKSTGMTSRSGTDVDAANLRETFRNLKYE
VRNKNDLTREEIVELMRDVSKEDHSKRSSFVCVLLSHGEEGIIFGTNGPV
DLKKITNFFRGDRCRSLTGKPKLFIIQACRGTELDCGIETDSGVDDDMAC
HKIPVEADFLYAYSTAPGYYSWRNSKDGSWFIQSLCAMLKQYADKLEFMH
ILTRVNRKVATEFESFSFDATFHAKKQIPGCFNFLTKELYFYH
SEQ ID NO: 50
>RIPK3-FKBP(F36V)-FKBP-F36V)
MSCVKLWPSGAPAPLVSIEELENQELVGKGGFGTVFRAQHRKWGYDVAVK
IVNSKAISREVKAMASLDNEFVLRLEGVIEKVNWDQDPKPALVTKFMENG
SLSGLLQSQCPRPWPLLCRLLKEVVLGMFYLHDQNPVLLHRDLKPSNVLL
DPELHVKLADFGLSTFQGGSQSGTGSGEPGGTLGYLAPELFVNVNRKAST
ASDVYSFGILMWAVLAGREVELPTEPSLVYEAVCNRQNRPSLAELPQAGP
ETPGLEGLKELMQLCWSSEPKDRPSFQECLPKTDEVFQMVENNMNAAVST
VKDFLSQLRSSNRRFSIPESGQGGTEMDGFRRTIENQHSRNDVMVSEWLN
KLNLEEPPSSVPKKCPSLTKRSRAQEEQVPQAWTAGTSSDSMAQPPQTPE
TSTFRNQMPSPTSTGTPSPGPRGNQGAERQGMNWSCRTPEPNPVTGRPLV
NIYNMLEGVQVETISPGDGRTFPKRGQTCVVHYTGMLEDGKKVDSSRDRN
KPFKFMLGKQEVIRGWEEGVAQMSVGQRAKLTISPDYAYGATGHPGIIPP
HATLVFDVELLKLEGGSGRLEGVQVETISPGDGRTFPKRGQTCVVHYTGM
LEDGKKVDSSRDRNKPFKFMLGKQEVIRGWEEGVAQMSVGQRAKLTISPD
YAYGATGHPGIIPPHATLVFDVELLKLE
SEQ ID NO: 51
>MLKL-FKBP(F36V)-FKBP-F36V)
MENLKHIITLGQVIHKRCEEMKYCKKQCRRLGHRVLGLIKPLEMLQDQGK
RSVPSEKLTTAMNRFKAALEEANGEIEKFSNRSNICRFLTASQDKILFKD
VNRKLSDVWKELSLLLQVEQRMPVSPISQGASWAQEDQQDADEDRRAFQM
LRRDNEKIEASLRRLEINMKEIKETLRQYLPPKCMQEIPQEQIKEIKKEQ
LSGSPWILLRENEVSTLYKGEYHRAPVAIKVFKKLQAGSIAIVRQTFNKE
IKTMKKFESPNILRIFGICIDETVTPPQFSIVMEYCELGTLRELLDREKD
LTLGKRMVLVLGAARGLYRLHHSEAPELHGKIRSSNFLVTQGYQVKLAGF
ELRKTQTSMSLGTTREKTDRVKSTAYLSPQELEDVFYQYDVKSEIYSFGI
VLWEIATGDIPFQGCNSEKIRKLVAVKRQQEPLGEDCPSELREIIDECRA
HDPSVRPSVDEILKKLSTFSKGSMLEGVQVETISPEGGRTFPKRGQTCVV
HYTGMLEDGKKVDSSRDRNKPFKFMLGKQEVIRGWEEGVAQMSVGQRAKL
TISPDYAYGATGHPGIIPPHATLVFDVELLKLETGSGMLEGVQVETISPG
DGRTFPKRGQTCVVHYTGMLEDGKKVDSSRDRNKPFKFMLGKQEVIRGWE
EGVAQMSVGQRAKLTISPDYAYGATGHPGIIPPHATLVFDVELLKLE
SEQ ID NO: 52
>APAF1-FKBP(F36V)-FKBP-F36V)
MDAKARNCLLQHREALEKDIKTSYIMDHMISDGFLTISEEEKVRNEPTQQ
QRAAMLIKMILKKDNDSYVSFYNALLHEGYKDLAALLHDGIPVVSSSSGK
DSVSGITSGSMLEGVQVETISPEGGRTFPKRGQTCVVHYTGMLEDGKKVD
SSRDRNKPFKFMLGKQEVIRGWEEGVAQMSVGQRAKLTISPDYAYGATGH
PGIIPPHATLVFDVELLKLETGSGMLEGVQVETISPGDGRTFPKRGQTCV
VHYTGMLEDGKKVDSSRDRNKPFKFMLGKQEVIRGWEEGVAQMSVGQRAK
LTISPDYAYGATGHPGIIPPHATLVFDVELLKLE
SEQ ID NO: 53
>DNCR2-Casp-1
MSSDEEEARELIERAKEAAERAQEAAERTGDPRVRELARELKRLAQEAAE
EVKRDPSSSDVNEALKLIVEAIEAAVDALEAAERTGDPEVRELARELVRL
AVEAAEEVQRNPSSSDVNEALHSIVYAIEAAIFALEAAERTGDPEVRELA
RELVRLAVEAAEEVQRNPSSRNVEHALMRIVLAIYLAEENLREAEESGDP
EKREKARERVREAVERAEEVQRDPSGWLNHTGSGGGSGNPAMPTSSGSEG
NVKLCSLEEAQRIWKQKSAEIYPIMDKSSRTRLALIICNEEFDSIPRRTG
AEVDITGMTMLLQNLGYSVDVKKNLTASDMTTELEAFAHRPEHKTSDSTF
LVFMSHGIREGICGKKHSEQVPDILQLNAIFNMLNTKNCPSLKDKPKVII
IQACRGDSPGVVWFKDSVGVSGNLSLPTTEEFEDDAIKKAHIEKDFIAFC
SSTPDNVSWRHPTMGSVFIGRLIEHMQEYACSCDVEEIFRKVRFSFEQPD
GRAQMPTTERVTLTRCFYLFPGH
SEQ ID NO: 54
>DNCR2-Casp-4
MSSDEEEARELIERAKEAAERAQEAAERTGDPRVRELARELKRLAQEAAE
EVKRDPSSSDVNEALKLIVEAIEAAVDALEAAERTGDPEVRELARELVRL
AVEAAEEVQRNPSSSDVNEALHSIVYAIEAAIFALEAAERTGDPEVRELA
RELVRLAVEAAEEVQRNPSSRNVEHALMRIVLAIYLAEENLREAEESGDP
EKREKARERVREAVERAEEVQRDPSGWLNHTGSGGGSGEAGPPESGESTD
ALKLCPHEEFLRLCKERAEEIYPIKERNNRTRLALIICNTEFDHLPPRNG
ADFDITGMKELLEGLDYSVDVEENLTARDMESALRAFATRPEHKSSDSTF
LVLMSHGILEGICGTVHDEKKPDVLLYDTIFQIFNNRNCLSLKDKPKVII
VQACRGANRGELWVRDSPASLEVASSQSSENLEEDAVYKTHVEKDFIAFC
SSTPHNVSWRDSTMGSIFITQLITCFQKYSWCCHLEEVFRKVQQSFETPR
AKAQMPTIERLSMTRYFYLFPGN
SEQ ID NO: 55
>DNCR2-Casp-3
MSSDEEEARELIERAKEAAERAQEAAERTGDPRVRELARELKRLAQEAAE
EVKRDPSSSDVNEALKLIVEAIEAAVDALEAAERTGDPEVRELARELVRL
AVEAAEEVQRNPSSSDVNEALHSIVYAIEAAIFALEAAERTGDPEVRELA
RELVRLAVEAAEEVQRNPSSRNVEHALMRIVLAIYLAEENLREAEESGDP
EKREKARERVREAVERAEEVQRDPSGWLNHTGSGGGSGMENTENSVDSKS
IKNLEPKIIHGSESMDSGISLDNSYKMDYPEMGLCIIINNKNFHKSTGMT
SRSGTDVDAANLRETFRNLKYEVRNKNDLTREEIVELMRDVSKEDHSKRS
SFVCVLLSHGEEGIIFGTNGPVDLKKITNFFRGDRCRSLTGKPKLFIIQA
CRGTELDCGIETDSGVDDDMACHKIPVEADFLYAYSTAPGYYSWRNSKDG
SWFIQSLCAMLKQYADKLEFMHILTRVNRKVATEFESFSFDATFHAKKQI
PCIVSMLTKELYFYH
SEQ ID NO: 56
>DNCR2-Casp-3-Casp9interface
MSSDEEEARELIERAKEAAERAQEAAERTGDPRVRELARELKRLAQEAAE
EVKRDPSSSDVNEALKLIVEAIEAAVDALEAAERTGDPEVRELARELVRL
AVEAAEEVQRNPSSSDVNEALHSIVYAIEAAIFALEAAERTGDPEVRELA
RELVRLAVEAAEEVQRNPSSRNVEHALMRIVLAIYLAEENLREAEESGDP
EKREKARERVREAVERAEEVQRDPSGWLNHTGSGGGSGMENTENSVDSKS
IKNLEPKIIHGSESMDSGISLDNSYKMDYPEMGLCIIINNKNFHKSTGMT
SRSGTDVDAANLRETFRNLKYEVRNKNDLTREEIVELMRDVSKEDHSKRS
SFVCVLLSHGEEGIIFGTNGPVDLKKITNFFRGDRCRSLTGKPKLFIIQA
CRGTELDCGIETDSGVDDDMACHKIPVEADFLYAYSTAPGYYSWRNSKDG
SWFIQSLCAMLKQYADKLEFMHILTRVNRKVATEFESFSFDATFHAKKQI
PGCFNFLTKELYFYH
SEQ ID NO: 57
>RIPK3-DNCR2
MSCVKLWPSGAPAPLVSIEELENQELVGKGGFGTVFRAQHRKWGYDVAVK
IVNSKAISREVKAMASLDNEFVLRLEGVIEKVNWDQDPKPALVTKFMENG
SLSGLLQSQCPRPWPLLCRLLKEVVLGMFYLHDQNPVLLHRDLKPSNVLL
DPELHVKLADFGLSTFQGGSQSGTGSGEPGGTLGYLAPELFVNVNRKAST
ASDVYSFGILMWAVLAGREVELPTEPSLVYEAVCNRQNRPSLAELPQAGP
ETPGLEGLKELMQLCWSSEPKDRPSFQECLPKTDEVFQMVENNMNAAVST
VKDFLSQLRSSNRRFSIPESGQGGTEMDGFRRTIENQHSRNDVMVSEWLN
KLNLEEPPSSVPKKCPSLTKRSRAQEEQVPQAWTAGTSSDSMAQPPQTPE
TSTFRNQMPSPTSTGTPSPGPRGNQGAERQGMNWSCRTPEPNPVTGRPLV
NIYNSSDEEEARELIERAKEAAERAQEAAERTGDPRVRELARELKRLAQE
AAEEVKRDPSSSDVNEALKLIVEAIEAAVDALEAAERTGDPEVRELAREL
VRLAVEAAEEVQRNPSSSDVNEALHSIVYAIEAAIFALEAAERTGDPEVR
ELARELVRLAVEAAEEVQRNPSSRNVEHALMRIVLAIYLAEENLREAEES
GDPEKREKARERVREAVERAEEVQRDPSGWLNH
SEQ ID NO: 58
>MLKL-DNCR2
MENLKHIITLGQVIHKRCEEMKYCKKQCRRLGHRVLGLIKPLEMLQDQGK
RSVPSEKLTTAMNRFKAALEEANGEIEKFSNRSNICRFLTASQDKILFKD
VNRKLSDVWKELSLLLQVEQRMPVSPISQGASWAQEDQQDADEDRRAFQM
LRRDNEKIEASLRRLEINMKEIKETLRQYLPPKCMQEIPQEQIKEIKKEQ
LSGSPWILLRENEVSTLYKGEYHRAPVAIKVFKKLQAGSIAIVRQTFNKE
IKTMKKFESPNILRIFGICIDETVTPPQFSIVMEYCELGTLRELLDREKD
LTLGKRMVLVLGAARGLYRLHHSEAPELHGKIRSSNFLVTQGYQVKLAGF
ELRKTQTSMSLGTTREKTDRVKSTAYLSPQELEDVFYQYDVKSEIYSFGI
VLWEIATGDIPFQGCNSEKIRKLVAVKRQQEPLGEDCPSELREIIDECRA
HDPSVRPSVDEILKKLSTFSKGSSSDEEEARELIERAKEAAERAQEAAER
TGDPRVRELARELKRLAQEAAEEVKRDPSSSDVNEALKLIVEAIEAAVDA
LEAAERTGDPEVRELARELVRLAVEAAEEVQRNPSSSDVNEALHSIVYAI
EAAIFALEAAERTGDPEVRELARELVRLAVEAAEEVQRNPSSRNVEHALM
RIVLAIYLAEENLREAEESGDPEKREKARERVREAVERAEEVQRDPSGWL
NH
SEQ ID NO: 59
>APAF1-DNCR2
MDAKARNCLLQHREALEKDIKTSYIMDHMISDGFLTISEEEKVRNEPTQQ
QRAAMLIKMILKKDNDSYVSFYNALLHEGYKDLAALLHDGIPVVSSSSGK
DSVSGITSGSSSDEEEARELIERAKEAAERAQEAAERTGDPRVRELAREL
KRLAQEAAEEVKRDPSSSDVNEALKLIVEAIEAAVDALEAAERTGDPEVR
ELARELVRLAVEAAEEVQRNPSSSDVNEALHSIVYAIEAAIFALEAAERT
GDPEVRELARELVRLAVEAAEEVQRNPSSRNVEHALMRIVLAIYLAEENL
REAEESGDPEKREKARERVREAVERAEEVQRDPSGWLNH
SEQ ID NO: 60
>APAF1-NS3a-DNCR2
MDAKARNCLLQHREALEKDIKTSYIMDHMISDGFLTISEEEKVRNEPTQQ
QRAAMLIKMILKKDNDSYVSFYNALLHEGYKDLAALLHDGIPVVSSSSGK
DSVSGITSGSMKKKGSVVIVGRINLSGDTAYAQQTRGEEGCQEMSQTGRD
KNQVEGEVQIVSTATQTFLATSINGVLWTVYHGAGTRTIASPKGPVTQMY
TNVDKDLVGWQAPQGSRSLTPCTCGSSDLYLVTRHADVIPVRRRGDSRGS
LLSPRPISYLKGSAGGPLLCPAGHAVGIFRAAVSTRGVAKAVDFIPVESL
ETTMRSPTGSGGGSSSDEEEARELIERAKEAAERAQEAAERTGDPRVREL
ARELKRLAQEAAEEVKRDPSSSDVNEALKLIVEAIEAAVDALEAAERTGD
PEVRELARELVRLAVEAAEEVQRNPSSSDVNEALHSIVYAIEAAIFALEA
AERTGDPEVRELARELVRLAVEAAEEVQRNPSSRNVEHALMRIVLAIYLA
EENLREAEESGDPEKREKARERVREAVERAEEVQRDPSGWLNH
SEQ ID NO: 61
>RIPK3-NS3a-P2a-RIPK3-DNCR2
MSCVKLWPSGAPAPLVSIEELENQELVGKGGFGTVFRAQHRKWGYDVAVK
IVNSKAISREVKAMASLDNEFVLRLEGVIEKVNWDQDPKPALVTKFMENG
SLSGLLQSQCPRPWPLLCRLLKEVVLGMFYLHDQNPVLLHRDLKPSNVLL
DPELHVKLADFGLSTFQGGSQSGTGSGEPGGTLGYLAPELFVNVNRKAST
ASDVYSFGILMWAVLAGREVELPTEPSLVYEAVCNRQNRPSLAELPQAGP
ETPGLEGLKELMQLCWSSEPKDRPSFQECLPKTDEVFQMVENNMNAAVST
VKDFLSQLRSSNRRFSIPESGQGGTEMDGFRRTIENQHSRNDVMVSEWLN
KLNLEEPPSSVPKKCPSLTKRSRAQEEQVPQAWTAGTSSDSMAQPPQTPE
TSTFRNQMPSPTSTGTPSPGPRGNQGAERQGMNWSCRTPEPNPVTGRPLV
NIYNMKKKGSVVIVGRINLSGDTAYAQQTRGEEGCQETSQTGRDKNQVEG
EVQIVSTATQTFLATSINGVLWTVYHGAGTRTIASPKGPVTQMYTNVDKD
LVGWQAPQGSRSLTPCTCGSSDLYLVTRHADVIPVRRRGDSRGSLLSPRP
ISYLKGSAGGPLLCPAGHAVGIFRAAVSTRGVAKAVDFIPVESLETTMRS
PGSGEGRGSLLTCGDVEENPGPMSCVKLWPSGAPAPLVSIEELENQELVG
KGGFGTVFRAQHRKWGYDVAVKIVNSKAISREVKAMASLDNEFVLRLEGV
IEKVNWDQDPKPALVTKFMENGSLSGLLQSQCPRPWPLLCRLLKEVVLGM
FYLHDQNPVLLHRDLKPSNVLLDPELHVKLADFGLSTFQGGSQSGTGSGE
PGGTLGYLAPELFVNVNRKASTASDVYSFGILMWAVLAGREVELPTEPSL
VYEAVCNRQNRPSLAELPQAGPETPGLEGLKELMQLCWSSEPKDRPSFQE
CLPKTDEVFQMVENNMNAAVSTVKDFLSQLRSSNRRFSIPESGQGGTEMD
GFRRTIENQHSRNDVMVSEWLNKLNLEEPPSSVPKKCPSLTKRSRAQEEQ
VPQAWTAGTSSDSMAQPPQTPETSTFRNQMPSPTSTGTPSPGPRGNQGAE
RQGMNWSCRTPEPNPVTGRPLVNIYNSSDEEEARELIERAKEAAERAQEA
AERTGDPRVRELARELKRLAQEAAEEVKRDPSSSDVNEALKLIVEAIEAA
VDALEAAERTGDPEVRELARELVRLAVEAAEEVQRNPSSSDVNEALHSIV
YAIEAAIFALEAAERTGDPEVRELARELVRLAVEAAEEVQRNPSSRNVEH
ALMRIVLAIYLAEENLREAEESGDPEKREKARERVREAVERAEEVQRDPS
GWLNH
SEQ ID NO: 62

TABLE B
Sequences

| | SEQ ID | |
|---|---|---|
| Name | NO: | Sequence |
| CD28 | | MLRLLLALNL FPSIQVTGNK ILVKQSPMLV AYDNAVNLSC |
| | | KYSYNLFSRE FRASLHKGLD |
| | | SAVEVCVVYG NYSQQLQVYS KTGFNCDGKL GNESVTFYLQ |
| | | NLYVNQTDIY FCKIEVMYPP |
| | | PYLDNEKSNG TIIHVKGKHL CPSPLFPGPS KPFWVLVVVG |
| | | GVLACYSLLV TVAFIIFWVR |
| | | SKRSRLLHSD YMNMTPRRPG PTRKHYQPYA PPRDFAAYRS |
| | | (UniProtKB Accession No. P10747-1) |
| CD28 | | MLRLLLALNL FPSIQVTGKH LCPSPLFPGP SKPFWVLVVV |
| | | GGVLACYSLL VTVAFIIFWV |
| | | RSKRSRLLHS DYMNMTPRRP GPTRKHYQPY APPRDFAAYR S |
| | | (UniProtKB Accession No. P10747-2) |
| CD28 | | MLRLLLALNL FPSIQVTGNK ILVKQSPMLV AYDNAVNLSY |
| | | NEKSNGTIIH VKGKHLCPSP |
| | | LFPGPSKPFW VLVVVGGVLA CYSLLVTVAF IIFWVRSKRS |
| | | RLLHSDYMNM TPRRPGPTRK |
| | | HYQPYAPPRD FAAYRS |
| | | (UniProtKB Accession No. P10747-3) |
| CD28 | | MLRLLLALNL FPSIQVTGNK ILVKQSPMLV AYDNAVNLSW |
| | | KHLCPSPLFP GPSKPFWVLV |
| | | VVGGVLACYS LLVTVAFIIF WVRSKRSRLL HSDYMNMTPR |
| | | RPGPTRKHYQ PYAPPRDFAA |
| | | YRS |
| | | (UniProtKB Accession No. P10747-4) |
| CD28 | | MLRLLLALNL FPSIQVTGNK ILVKQSPMLV AYDNAVNLSY |
| | | NEKSNGTIIH VKGEE |
| | | (UniProtKB Accession No. P10747-5) |
| CD28 | | MLRLLLALNL FPSIQVTGNK ILVKQSPMLV AYDNAVNLSY |
| | | NEKSNGTIIH VKGKHLCPSP |
| | | LFPGPSKPYA PPRDFAAYRS |
| | | (UniProtKB Accession No. P10747-6) |
| CD28 | | MPCGLSALIM CPKGMVAVVV AVDDGDSQAL AGNKILVKQS |
| | | PMLVAYDNAV NLSCKYSYNL |
| | | FSREFRASLH KGLDSAVEVC VVYGNYSQQL QVYSKTGFNC |
| | | DGKLGNESVT FYLQNLYVNQ |
| | | TDIYFCKIEV MYPPPYLDNE KSNGTIIHVK GKHLCPSPLG |
| | | PGPSKPFWVL VVVGGVLACY |
| | | SLLVTVAFII FWVRSKRSRL LHSDYMNMTP RRPGPTRKHY |
| | | QPYAPPRDFA AYRS |
| | | (UniProtKB Accession No. P10747-7) |
| 4-1BB | | MGNSCYNIVA TLLLVLNFER TRSLQDPCSN CPAGTFCDNN |
| | | RNQICSPCPP NSFSSAGGQR |
| | | TCDICRQCKG VFRTRKECSS TSNAECDCTP GFHCLGAGCS |
| | | MCEQDCKQGQ ELTKKGCKDC |
| | | CFGTFNDQKR GICRPWTNCS LDGKSVLVNG TKERDVVCGP |
| | | SPADLSPGAS SVTPPAPARE |
| | | PGHSPQIISF FLALTSTALL FLLFFLTLRF SVVKRGRKKL |
| | | LYIFKQPFMR PVQTTQEEDG |
| | | CSCRFPEEEE GGCEL |
| | | (UniProtKB Accession No. Q07011-1) |
| CD27 | | MARPHPWWLC VLGTLVGLSA TPAPKSCPER HYWAQGKLCC |
| | | QMCEPGTFLV KDCDQHRKAA |
| | | QCDPCIPGVS FSPDHHTRPH CESCRHCNSG LLVRNCTITA |
| | | NAECACRNGW QCRDKECTEC |
| | | DPLPNPSLTA RSSQALSPHP QPTHLPYVSE MLEARTAGHM |
| | | QTLADFRQLP ARTLSTHWPP |
| | | QRSLCSSDFI RILVIFSGMF LVFTLAGALF LHQRRKYRSN |
| | | KGESPVEPAE PCHYSCPREE |
| | | EGSTIPIQED YRKPEPACSP |
| | | (UniProt Accession No. P26842-1) |
| CD3 | | MKWKALFTAA ILQAQLPITE AQSFGLLDPK LCYLLDGILF |
| zeta | | IYGVILTALF LRVKFSRSAD |
| | | APAYQQGQNQ LYNELNLGRR EEYDVLDKRR GRDPEMGGKP |
| | | QRRKNPQEGL YNELQKDKMA |
| | | EAYSEIGMKG ERRRGKGHDG LYQGLSTATK DTYDALHMQA LPPR |
| | | (UniProtKB Accession No. P20963-1) |
| CD3 | | MKWKALFTAA ILQAQLPITE AQSFGLLDPK LCYLLDGILF |
| zeta | | IYGVILTALF LRVKFSRSAD |
| | | APAYQQGQNQ LYNELNLGRR EEYDVLDKRR GRDPEMGGKP |
| | | RRKNPQEGLY NELQKDKMAE |
| | | AYSEIGMKGE RRRGKGHDGL YQGLSTATKD TYDALHMQAL PPR |
| | | (UniProtKB Accession No. P20963-3) |
| ICOS | | MKSGLWYFFLFCLRIKVLTGEINGSANYEMFIFHNGGVQILCKYPDIVQQFKM |
| | | QLLKGGQ |
| | | ILCDLTKTKGSGNTVSIKSLKFCHSQLSNNSVSFFLYNLDHSHANYYFCNLSI |
| | | FDPPPFK |
| | | VTLTGGYLHIYESQLCCQLKFWLPIGCAAFVVVCILGCILICWLTKKKYSSSV |
| | | HDPNGEY |
| | | MFMRAVNTAKKSRLTDVTL |
| | | (UniProtKB Accession No. Q9Y6W8-1) |
| ICOS | | MKSGLWYFFLFCLRIKVLTGEINGSANYEMFIFHNGGVQILCKYPDIVQQFKM |
| | | QLLKGGQ |
| | | ILCDLTKTKGSGNTVSIKSLKFCHSQLSNNSVSFFLYNLDHSHANYYFCNLSI |
| | | FDPPPEK |
| | | VTLTGGYLHIYESQLCCQLKFWLPIGCAAFVVVCILGCILICWLTKKM |
| | | (UniProtKB Accession No. Q9Y6W8-2) |
| OX40 | | MERVQPLEENVGNAARPRFERNKLLLVASVIQGLGLLLCFTYICLHFSALQVS |
| | | HRYPRIQ |
| | | SIKVQFTEYKKEKGFILTSQKEDEIMKVQNNSVIINCDGFYLISLKGYFSQEV |
| | | NISLHYQ |
| | | KDEEPLFQLKKVRSVNSLMVASLTYKDKVYLNVTTDNTSLDDFHVNGGELILI |
| | | HQNPGEF |
| | | CVL |
| | | (UniProtKB Accession No. P23510-1) |
| OX40 | | MVSHRYPRIQSIKVQFTEYKKEKGFILTSQKEDEIMKVQNNSVIINCDGFYLI |
| | | SLKGYFS |
| | | QEVNISLHYQKDEEPLFQLKKVRSVNSLMVASLTYKDKVYLNVTTDNTSLDDF |
| | | HVNGGEL |
| | | ILIHQNPGEFCVL |
| | | (UniProtKB Accession No. P23510-2) |

TABLE C
Sequences

>LeucineZipper-GCN4 (dimer)
QRMKQLEDKVEELLSKNYHLENEVARLKKLVGDAAR
>pRO2.5 (trimer)
SEYEIRKALEELKASTAELKRATASLRASTEELKKNPSEDALVENNRLIVEHNAIIVENNR
IIAAVLELIVRAIK
>2L4HC2_23 (dimer)
TRTEIIRELERSLREQEELAKRLKELLRELERLQREGSSDEDVRELLREIKELVEEIEKLAR
EQKYLVEELKRQD
>5H2LD_10_A (pentamer)
TRRKQEMKRLKKEMEKIREETEEVKKEIEESKKRPQSESAKNLILIMQLLINQIRLLALQI
RMLALQLQE
>6H2LD_8_A (hexamer)
TEDEIRKLRKLLEEAEKKLKKLEDKTRRSEEISKTDDDPKAQSLQLIAESLMLIAESLLIIA
ISLLLSSRNG
>ANR
GELDELVYLLDGPGYDPIHSDVVTRGGSHLFNF
>DNCR2
SSDEEEARELIERAKEAAERAQEAAERTGDPRVRELARELKRLAQEAAEEVKRDPSSSD
VNEALKLIVEAIEAAVDALEAAERTGDPEVRELARELVRLAVEAAEEVQRNPSSSDVNE
ALHSIVYAIEAAIFALEAAERTGDPEVRELARELVRLAVEAAEEVQRNPSSRNVEHALM
RIVLAIYLAEENLREAEESGDPEKREKARERVREAVERAEEVQRDPSGWLNH
>DNCR2_1
DDNEAEKLIREAEEAAKDAEEAAERTGDPEVRELARELVRLAKEAAEEVQRNPSSSDVN
EALHSIVYAIEAAIFALEAAERTGDPEVRELARELVRLAVEAAEEVQRNPSSRNVEHALM
RIVLAIYLAEENLRE
>DNCR2_2
EERELARELERLAKEAAEEVQRNPSSSDVNEALHSIEYAREAAEFAREAAERTGDPEVRE
LARELVRLAVEAAEEVQRNPSSRNVEHALMRIVLAIYLAEENLRE
>DNCR2_3
DENEAEHSRRYAKEAAEFAREAAERTGDPEVRELARELERLAREAAEEVQRNPSSRNVE
HALMRIVLAIYLAEENLRE
>DNCR2_4
DENELEHIRRYLKEILEFVREVVERTGDEHIRRLLEELERLVKKVIEELKRRPDLHHNEHA
FMRLVLAVYLAEENLRE
>DNCR2_5
DENELEHIRRYLKEVLEFVREILERTGDEEVKKLIEELERLVKKIEEELKRDPRRHHNEHA
VMRLVLAVYLLEENLRE
>DNCR2_6
DEEELHKRVEELWKELHELVRRHSHDSDLNELLHIIEYLREVLEFVREVLERTGDEHIKR
LLHELVKRIVETIEELKRNSHHSNNEHAVMRIVLAVYLAEENLRE
>DNCR2_7
DEEELERRLEELWRRIKERVERHSHDSDLNELLHIIEYLREVVEFVREVVERTGDEHIKRL
LLELIREIVEVIEKLERNLHDSNNEHAVMRIVLAVYLAEENLRE
>DNCR2_8
EVEEVLKDLEEAIERLHDHEINRLAKEVLKILRKRDDHSIIYAVEVMIFVIEVLERTGDEK
VRELIRELAEIIRRKGGTDGAVMRLVLAIYLLEENLRE
>DNCR2_9
ELEEVLKDLEEALERLRNEELRRLIEEIAELLRRRDDHSIIYMIEALIFVIEAVERTGDERIE
KLLEELIKRIRNGDDDSAIMRIVLAVYLIEENLRE
>DNCR2_10
EAEEAAKDAEEAAERLHDPEVRELARELVRLAKETTHEDGLHSIVYAIEAAIFALEAAER
TGDPEVRELARELVRLARNGDHSGALMRIVLAIYLAEENLRE
>DNCR2_11
EAEEAAKDAEEAAERLHDPEVRELARELVRLAKERDDHSIVYAIEAAIFALEAAERTGD
PEVRELARELVRLAREGDHSSALMRIVLAIYLAEENLRE
>DNCR2_12
DDNEAEKLIREAEEVLKDIEEILERLHDEEIIRLVEEVQKILKELLERVHKHPHDSDLNELL
HIIFYMMEVLIFVLEVLERTGDEEIRKLVWHIVRIILEILEELRRNPKHSNNEHALMRLLL
AIYLLEENLRE
>DNCR2_13
EAEEAAKDAEEAAERLHDPEVRELARELVRLAKEVDDHSIVYAIEAAIFALEAAERTGD
PEVRELARELVRLAVEAAEEVQRNPSSRNVEHALMRIVLAIYLAEENLRE
>DNCR2_14
EFEEVLKDLEEVVERLHNEEIRRLLERLIELLRRVRDHSFIYLVEAVIFVLEVLERTGDHR
VEELLEKLVRLVIEVLEELERHPHKENNEHAAMRFVLAVYLIEENLRE
>DNCR2_15
EWEEVLKDLEEALERLHDEEIRRLLERVVELLREDTDHSIVYLIEALIFVIEAVERTGDERI
HRLLEELVRRIIKVIEELHRHPHKENNEHAAMRFVLAVYLIEENLRE
>DNCR2_16
EERELARELERLARERRDHSIEYAREAAEFAREAAERTGDPEVRELARELVRLARNGSD
EHALMRIVLAIYLAEENLRE
>DNCR2_17
EERELARELERLAKEAAEEVQRNPSSSDVNEALHSIEYAREAAEFAREAAERTGDPEVRE
LARELVRLARNGSDEHALMRIVLAIYLAEENLRE
>DNCR2_18
DEEEIRKRIEEQWRRDTDHSWEYLREVAEFAREILERTGDERIKELVLRILELIENGTDDH
AIMRLVLALYLIEENLRE
>DNCR2_19
DEEELREEVEHLWREDTDHSWEYLREALEFLREIVERTGDERLRELLKKALELVRHGTD
GHAIMRLVLAAYLLEENLRE
>DNCR2_20
YAKEAAEFAREAAERTGDPEVRELARELERLARRGSSEHALMRIVLAIYLAEENLRE
>DNCR2_21
YAKEVAEFVREVWERTGDERVEELLKKLEELRRNGSDEHALMRLVLAVYLIEENLRE
>DNCR2_22
YWKEVAEFLREVLERTGDEEVKKLLEEFEELRRRGSDEHALMRLVLAVYLIEENLRE
>DNCR2_23
DEEELERKIEEQVRKLREEVERRSHDSDLNEALHSMEYLREIAEFAREIVERTGDEELRK
LLKEILRLIHENPEDVGHAIMRLILALYLLEENLRE
>DNCR2_24
DDNEAEKLIREAEEAAKDAEEAAERTGDPEVRELARELVRLAKEAAEEVQRNPSSSDVN
EALHSIVYAIEAAIFALEAAERTGDPEVRELARELVRLAVEAAEEVQRNPSSRNVEHALM
RIVLAIYLAEENLREAEESGDPEKREKARERVREAVERAEEVQRD
>DNCR2_25
EERELARELERLAKEAAEEVQRNPSSSDVNEALHSIEYAREAAEFAREAAERTGDPEVRE
LARELVRLAVEAAEEVQRNPSSRNVEHALMRIVLAIYLAEENLREAEESGDPEKREKAR
ERVREAVERAEEVQRD
>DNCR2_26
DENEAEHSRRYAKEAAEFAREAAERTGDPEVRELARELERLAREAAEEVQRNPSSRNVE
HALMRIVLAIYLAEENLREAEESGDPEKREKARERVREAVERAEEVQRD
>DNCR2_27
DENELEHIRRYLKEILEFVREVVERTGDEHIRRLLEELERLVKKVIEELKRRPDLHHNEHA
FMRLVLAVYLAEENLREAEESGDPEKREKARERVREAVERAEEVQRD
>DNCR2_28
DENELEHIRRYLKEVLEFVREILERTGDEEVKKLIEELERLVKKIEEELKRDPRRHHNEHA
VMRLVLAVYLLEENLREAEESGDPEKREKARERVREAVERAEEVQRD
>DNCR2_29
DEEELHKRVEELWKELHELVRRHSHDSDLNELLHIIEYLREVLEFVREVLERTGDEHIKR
LLHELVKRIVETIEELKRNSHHSNNEHAVMRIVLAVYLAEENLREAEESGDPEKREKARE
RVREAVERAEEVQRD
>DNCR2_30
DEEELERRLEELWRRIKERVERHSHDSDLNELLHIIEYLREVVEFVREVVERTGDEHIKRL
LLELIREIVEVIEKLERNLHDSNNEHAVMRIVLAVYLAEENLREAEESGDPEKREKARER
VREAVERAEEVQRD
>DNCR2_31
DDNEAEKLIREAEEVLKDIEEILERLHDEEIIRLVEEVQKILKELLERVHKHPHDSDLNELL
HIIFYMMEVLIFVLEVLERTGDEEIRKLVWHIVRIILEILEELRRNPKHSNNEHALMRLLL
AIYLLEENLREAEESGDPEKREKARERVREAVERAEEVQRD
>DNCR2_32
EAEEAAKDAEEAAERLHDPEVRELARELVRLAKEVDDHSIVYAIEAAIFALEAAERTGD
PEVRELARELVRLAVEAAEEVQRNPSSRNVEHALMRIVLAIYLAEENLREAEESGDPEK
REKARERVREAVERAEEVQRD
>DNCR2_33
EFEEVLKDLEEVVERLHNEEIRRLLERLIELLRRVRDHSFIYLVEAVIFVLEVLERTGDHR
VEELLEKLVRLVIEVLEELERHPHKENNEHAAMRFVLAVYLIEENLREAEESGDPEKREK
ARERVREAVERAEEVQRD
>DNCR2_34
EWEEVLKDLEEALERLHDEEIRRLLERVVELLREDTDHSIVYLIEALIFVIEAVERTGDERI
HRLLEELVRRIIKVIEELHRHPHKENNEHAAMRFVLAVYLIEENLREAEESGDPEKREKA
RERVREAVERAEEVQRD
>DNCR2-3rep
TSSDEEEARELIEEAKEAAEDAQEAAERTGDPEVRELARELKRLAQEAAEEVQRNPSSSD
VNEALHSIVYAIEAAIFALEAAERTGDPEVRELARELVRLAVEAAEEVQRNPSSRNVEHA
LMRIVLAIYLAEENLREAEESGDPEKREKARERVREAVERAEEVQRDPSGWLNH
>GNCR1
DIEKLCKKAEEEAKEAQEKADELRQRHPDSQAAEDAEDLANLAVAAVLTACLLAQEHP
NADIAKLCIKAASEAAEAASKAAELAQRHPDSQAARDAIKLASQAARAVILAIMLAAEN
PNADIAKLCIKAASEAAEAASKAAELAQRHPDSQAARDAIKLASQAAEAVERAIWLAAE
NPNADIAKKCIKAASEAAEEASKAAEEAQRHPDSQKARDEIKEASQKAEEVKERCKS
>GNCR1-3rep
DIEKLCKKAEEEAKEAQEKADELRQRHPDSQAAEDAEDLANLAVAAVLTACLLAQEHP
NADIAKLCIKAASEAAEAASKAAELAQRHPDSQAARDAIKLASQAARAVILAIMLAAEN
PNADIAKKCIKAASEAAEEASKAAEEAQRHPDSQKARDEIKEASQKAEEVERRCWRAA
EN
>G33
DIEKLCKKAEEEAKEEGNELAVAAVLTACLLAQEWPNSHIAKLCIKAASEAAERGDSQA
ARAVILAIMLAAENPNNEHAEECIRLAMEAARDGTSEHAERVERECWKHME
>G38
DIEKLCKKAEEEAKEVGDNLAVAAVLTACLLAQEHPNADIAKLCIKAASEAAESGDKQ
AARAVILAIMLAAENPNADIAKLCIKAASEAAEGSKHGWSQAAEAVERAIWLAAENPN
ADIAKKCIKAASEAAREGNSQKAEEVKERCKS
>P2a
GSGATNFSLLKQAGDVEENPGP
>T2a
GSGEGRGSLLTCGDVEENPG
>T2a-RFP-P2a
GSGEGRGSLLTCGDVEENPGPMVSKGEEDNMAIIKEFMRFKVHMEGSVNGHEFEIEGEG
EGRPYEGTQTAKLKVTKGGPLPFAWDILSPQFMYGSKAYVKHPADIPDYLKLSFPEGFK
WERVMNFEDGGVVTVTQDSSLQDGEFIYKVKLRGTNFPSDGPVMQKKTMGWEASSER
MYPEDGALKGEIKQRLKLKDGGHYDAEVKTTYKAKKPVQLPGAYNVNIKLDITSHNED
YTIVEQYERAEGRHSTGGMDELYKGSGATNFSLLKQAGDVEENPGP
>YB_TATA
TCTAGAGGGTATATAATGGGGGCCA
>human beta globin (huBG)
GAATTCAGGGCTGGGCATAAAAGTCAGGGCAGAGCCATCTATTGCTTACATTTGCTT
CTGACACAACTGTGTTCACTAGCAACCTCAAACAGACACC
>minIL2
TCGAGGACATTTTGACACCCCCATAATATTTTTCCAGAATTAACAGTATAAATTGCA
TCTCTTGTTCAAGAGTTCCCTATCACTCTCTTTAATCACTACTCACAGTAACCTCAAC
TCCTG
>minimalCMV (minCMV)
GAGGTAGGCGTGTACGGTGGGCGCCTATAAAAGCAGAGCTCGTTTAGTGAACCGTC
AGATCGCCTGGA
>TRE3G
ACGTATAAGCTTTGCTTATGTAAACCAGGGCGCCTATAAAAGAGTGCTGATTTTTTG
AGTAAACTTCAATTCCACAACACTTTTGTCTTATACCAACTTTCCGTACCACTTCCTA
CCCTCGTAAA
>MND
TGAAAGACCCCACCTGTAGGTTTGGCAAGCTAGGATCAAGGTTAGGAACAGAGAGA
CAGCAGAATATGGGCCAAACAGGATATCTGTGGTAAGCAGTTCCTGCCCCGGCTCA
GGGCCAAGAACAGTTGGAACAGCAGAATATGGGCCAAACAGGATATCTGTGGTAAG
CAGTTCCTGCCCCGGCTCAGGGCCAAGAACAGATGGTCCCCAGATGCGGTCCCGCC
CTCAGCAGTTTCTAGAGAACCATCAGATGTTTCCAGGGTGCCCCAAGGACCTGAAAT
GACCCTGTGCCTTATTTGAACTAACCAATCAGTTCGCTTCTCGCTTCTGTTCGCGCGC
TTCTGCTCCCCGAGCTCAATAAAAGAGCCCACAACCCCTCACTCGGC
>hPGK
GGGGTTGGGGTTGCGCCTTTTCCAAGGCAGCCCTGGGTTTGCGCAGGGACGCGGCTG
CTCTGGGCGTGGTTCCGGGAAACGCAGCGGCGCCGACCCTGGGTCTCGCACATTCTT
CACGTCCGTTCGCAGCGTCACCCGGATCTTCGCCGCTACCCTTGTGGGCCCCCCGGC
GACGCTTCCTGCTCCGCCCCTAAGTCGGGAAGGTTCCTTGCGGTTCGCGGCGTGCCG
GACGTGACAAACGGAAGCCGCACGTCTCACTAGTACCCTCGCAGACGGACAGCGCC
AGGGAGCAATGGCAGCGCGCCGACCGCGATGGGCTGTGGCCAATAGCGGCTGCTCA
GCAGGGCGCGCCGAGAGCAGCGGCCGGGAAGGGGCGGTGCGGGAGGCGGGGTGTG
GGGCGGTAGTGTGGGCCCTGTTCCTGCCCGCGCGGTGTTCCGCATTCTGCAAGCCTC
CGGAGCGCACGTCGGCAGTCGGCTCCCTCGTTGACCGAATCACCGACCTCTCTCCCC
AG
>NS3a1b
MAKGSVVIVGRINLSGDTAYSQQTRGLLGCIITSATGRDKNQVDGEVQVLSTATQSFLA
TCVNGVCWTVYHGAGSKTLAGPKGPITQMYTNVDQDLVGWPAPPGARSMTPCTCGSS
DLYLVTRHADVIPVRRRGDSRGSLLSPRPVSYLKGSAGGPLLCPSGHVVGIFRAAVCTR
GVAKAVDFIPVESMETTMRSP
>NS3aH1
MKKKGSVVIVGRINLSGDTAYSQQTRGLEGCQETSQTGRDKNQVEGEVQVVSTATQSF
LATSINGVLWTVYHGAGTRTIASPKGPVTQMYTNVDKDLVGWQAPQGSRSLTPCTCGS
SDLYLVTRHADVIPVRRRGDSRGSLLSPRPISYLKGSAGGPLLCPAGHAVGIFRAAVSTR
GVAKAVDFIPVESLETTMRSP
>NS3aoptS139A
KKKGSVVIVGRINLSGDTAYAQQTRGEEGCQETSQTGRDKNQVEGEVQIVSTATQTFLA
TSINGVLWTVYHGAGTRTIASPKGPVTQMYTNVDKDLVGWQAPQGSRSLTPCTCGSSD
LYLVTRHADVIPVRRRGDSRGSLLSPRPISYLKGSAGGPLLCPAGHAVGIFRAAVSTRGV
AKAVDFIPVESLETTMRSP
>P2a-T2a
GSGATNFSLLKQAGDVEENPGPGSGEGRGSLLTCGDVEENPGP
>T2a-P2a
GSGEGRGSLLTCGDVEENPGPGSGATNFSLLKQAGDVEENPGP
>CMV
CGTTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCATTG
ACGTCAATGGGTGGAGTATTTACGGTAAACTGCCCACTTGGCAGTACATCAAGTGTA
TCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTA
GTCATCGCTATTACCATGCTGATGCGGTTTTGGCAGTACATCAATGGGCGTGGATAG
CGGTTTGACTCACGGGGATTTCCAAGTCTCCACCCCATTGACGTCAATGGGAGTTTG
TTTTGGCACCAAAATCAACGGGACTTTCCAAAATGTCGTAACAACTCCGCCCCATTG
ACGCAAATGGGCGGTAGGCGTGTACGGTGGGAGGTCTATATAAGCAGAGCTGGTTT
AGTGAACCGTCAGAT
>CAG
ATTGACGTCAATAATGACGTATGTTCCCATAGTAACGCCAATAGGGACTTTCCATTG
ACGTCAATGGGTGGAGTATTTACGGTAAACTGCCCACTTGGCAGTACATCAAGTGTA
TCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTA
GTCATCGCTATTACCATGGTCGAGGTGAGCCCCACGTTCTGCTTCACTCTCCCCATCT
CCCCCCCCTCCCCACCCCCAATTTTGTATTTATTTATTTTTTAATTATTTTGTGCAGCG
ATGGGGGCGGGGGGGGGGGGGGGGCGCGCGCCAGGCGGGGGGGGGGGGGCGAG
GGGCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGA
AGCGCGCGGCGGGCGATCGATT
>SFFV
GTAACGCCATTTTGCAAGGCATGGAAAAATACCAAACCAAGAATAGAGAAGTTCAG
ATCAAGGGCGGGTACATGAAAATAGCTAACGTTGGGCCAAACAGGATATCTGCGGT
GAGCAGTTTCGGCCCCGGCCCGGGGCCAAGAACAGATGGTCACCGCAGTTTCGGCC
CCGGCCCGAGGCCAAGAACAGATGGTCCCCAGATATGGCCCAACCCTCAGCAGTTT
CTTAAGACCCATCAGATGTTTCCAGGCTCCCCCAAGGACCTGAAATGACCCTGCGCC
TTATTTGAATTAACCAATCAGCCTGCTTCTCGCTTCTGTTCGCGCGCTTCTGCTTCCC
GAGCTCTATAAAAGAGCTCACAACCCCTCACTCGGCGCGCCAGTCCTCCGACAGACT
GAGTCGCCCGGG
>EF1alpha
GGGCAGAGCGCACATCGCCCACAGTCCCCGAGAAGTTGGGGGGAGGGGTCGGCAAT
TGAACCGGTGCCTAGAGAAGGTGGCGCGGGGTAAACTGGGAAAGTGATGTCGTGTA
CTGGCTCCGCCTTTTTCCCGAGGGTGGGGGAGAACCGTATATAAGTGCAGTAGTCGC
CGTGAACGTTCTTTTTCGCAACGGGTTTGCCGCCAGAACACAG
>UBC
GGCCTCCGCGCCGGGTTTTGGCGCCTCCCGCGGGCGCCCCCCTCCTCACGGCGAGCG
CTGCCACGTCAGACGAAGGGCGCAGCGAGCGTCCTGATCCTTCCGCCCGGACGCTC
AGGACAGCGGCCCGCTGCTCATAAGACTCGGCCTTAGAACCCCAGTATCAGCAGAA
GGACATTTTAGGACGGGACTTGGGTGACTCTAGGGCACTGGTTTTCTTTCCAGAGAG
CGGAACAGGCGAGGAAAAGTAGTCCCTTCTCGGCGATTCTGCGGAGGGATCTCCGT
GGGGCGGTGAACGCCGATGATTATATAAGGACGCGCCGGGTGTGGCACAGCTAGTT
CCGTCGCAGCCGGGATTTGGGTCGCGGTTCTTGTTTGTGGATCGCTGTGATCGTCACT
TGGTGAGTAGCGGGCTGCTGGGCTGGCCGGGGCTTTCGTGGCCGCCGGGCCGCTCG
GTGGGACGGAAGCGTGTGGAGAGACCGCCAAGGGCTGTAGTCTGGGTCCGCGAGCA
AGGTTGCCCTGAACTGGGGGTTGGGGGGAGCGCAGCAAAATGGCGGCTGTTCCCGA
GTCTTGAATGGAAGACGCTTGTGAGGCGGGCTGTGAGGTCGTTGAAACAAGGTGGG
GGGCATGGTGGGCGGCAAGAACCCAAGGTCTTGAGGCCTTCGCTAATGCGGGAAAG
CTCTTATTCGGGTGAGATGGGCTGGGGCACCATCTGGGGACCCTGACGTGAAGTTTG
TCACTGACTGGAGAACTCGGTTTGTCGTCTGTTGCGGGGGCGGCAGTTATGGCGGTG
CCGTTGGGCAGTGCACCCGTACCTTTGGGAGCGCGCGCCCTCGTCGTGTCGTGACGT
CACCCGTTCTGTTGGCTTATAATGCAGGGTGGGGCCACCTGCCGGTAGGTGTGCGGT
AGGCTTTTCTCCGTCGCAGGACGCAGGGTTCGGGCCTAGGGTAGGCTCTCCTGAATC
GACAGGCGCCGGACCTCTGGTGAGGGGAGGGATAAGTGAGGCGTCAGTTTCTTTGG
TCGGTTTTATGTACCTATCTTCTTAAGTAGCTGAAGCTCCGGTTTTGAACTATGCGCT
CGGGGTTGGCGAGTGTGTTTTGTGAAGTTTTTTAGGCACCTTTTGAAATGTAATCATT
TGGGTCAATATGTAATTTTCAGTGTTAGACTAGTAAATTGTCCGCTAAATTCTGGCC
GTTTTTGGCTTTTTTGTTAGACATCGATT

TABLE 1

| Disease or | |
|---|---|
| disorder | Targeted Proteins (or antigens/epitopes thereof) |
| Autoimmune: | ABCC8, ADIPOQ, ADRB3, AGPAT2, AKT2, ALMS1, ANGPTL8, |
| | APPL1, AQP2, AVP, AVPR2, BANK1, BCAR1, BLK, BSCL2, C4A, |
| | C4B, CAPN10, CAV1, CAVIN1, CCR5, CD38, CDKAL1, CEL, |
| | CELA2A, CISD2, CLEC16A, CLPS, CR2, CTLA4, DCAF17, DMXL2, |
| | DNAJC3, DNASE1, DNASE1L3, DYRK1B, EIF2AK3, ENPP1, |
| | FCGR2B, FOXP3, GCGR, GCK, GLIS3, GSK3A, GSK3B, GYS1, |
| | HNF1B, HNF4A, IER3IP1, IFIH1, IL2RA, INPPL1, INS, INSR, IRF5, |
| | IRS1, ITGAM, KCNJ11, KCNQ1, KLF11, LEP, LIPE, LTK, MAFA, |
| | MAPK8IP1, MBP, MCF2L2, MT-ND1, NEUROD1, PAX4, PDCD1, |
| | PDX1, PLAGL1, PPARG, PPP1R15B, PPP1R3A, PTF1A, PTPN22, |
| | PTPRN, PTPRN2, RASGRP1, RETN, RFX6, SH2B3, SLC16A11, |
| | SLC16A13, SLC19A2, SLC2A4, SLC30A8, SSB, STAT3, STAT4, |
| | SUMO4, TBC1D4, TCF7L2, TLR5, TNFSF4, TREX1, TRMT10A, |
| | UCP1, UCP3, WFS1, XRCC5, XRCC6, ZFP57 |
| Blood | ACSL4, ADA, AK1, ALDOA, AMMECR1, ANK1, ATP11C, BPGM, |
| | BRCA1, BRCA2, BRIP1, CD59, CDAN1, CDIN1, CPOX, CYBA, |
| | CYBB, CYBC1, EPB41, EPB42, ERCC4, F8, F9, FANCA, FANCB, |
| | FANCC, FANCD2, FANCE, FANCF, FANCG, FANCI, FANCL, |
| | G6PD, GCLC, GPI, GSR, GSS, HBA1, HBA2, HBB, KCNE5, KCNN4, |
| | KLF1, LPIN2, MAD2L2, NCF1, NCF2, NCF4, PALB2, PGK1, |
| | PIEZO1, PKLR, RAD51, RAD51C, RFWD3, RHAG, RPL11, RPL15, |
| | RPL18, RPL26, RPL27, RPL35, RPL35A, RPL5, RPS10, RPS15A, |
| | RPS17, RPS19, RPS24, RPS26, RPS27, RPS28, RPS29, RPS7, |
| | SEC23B, SLC2A1, SLC4A1, SLX4, SPTA1, SPTB, TPI1, TSR2, |
| | UBE2T, XRCC2 |
| Bone | ANO5, BMP1, CA2, CLCN7, COL1A1, COL1A2, CREB3L1, CRTAP, |
| | FKBP10, IFITM5, IKBKG, LRP5, MBTPS2, MESD, MITF, OSTM1, |
| | P3H1, P4HB, PLEKHM1, PLOD2, PPIB, SEC24D, SERPINF1, |
| | SERPINH1, SNX10, SP7, SPARC, SPG7, TCIRG1, TENT5A, |
| | TMEM38B, TNFRSF11A, TNFSF11, WNT1 |
| Neurological | AARS1, AARS2, ABCA2, ABCA7, ABHD12, ACO2, ACOX1, |
| | ACTL6B, ADAM10, ADAM22, ADPRS, ADRA2B, ADSL, AFG3L2, |
| | AGTPBP1, AIFM1, ALDH18A1, ALDH7A1, ALG13, ALS2, AMPD2, |
| | ANG, ANO10, ANXA11, AP1S1, AP2M1, AP3B2, AP4B1, AP4E1, |
| | AP4M1, AP4S1, AP5Z1, APLP1, APOA1, APOE, APP, APTX, AR, |
| | ARHGEF9, ARL6IP1, ARSA, ARSB, ARV1, ARX, ASAH1, ASCC1, |
| | ATG5, ATIC, ATL1, ATL3, ATM, ATN1, ATP13A2, ATP1A1, |
| | ATP2A2, ATP2B3, ATP6AP2, ATP6V1A, ATP7A, ATXN1, ATXN10, |
| | ATXN2, ATXN3, ATXN7, B4GALNT1, BCKDK, BEAN1, BICD2, |
| | BRAT1, BSCL2, C12orf65, C19orf12, C1orf194, C9orf72, CACNA1A, |
| | CACNA1B, CACNA1D, CACNA1E, CACNA1G, CACNA1H, |
| | CACNA2D2, CACNB4, CAD, CAPN1, CASR, CCDC88A, CCDC88C, |
| | CCT5, CDK19, CDK5, CDKL5, CERS1, CHCHD10, CHCHD2, CHD2, |
| | CHMP1A, CHMP2B, CHP1, CHRNA2, CHRNA4, CHRNB2, CILK1, |
| | CLCN2, CLN3, CLN5, CLN6, CLN8, CLP1, CLPB, CNNM2, CNPY3, |
| | CNTN2, CNTNAP1, CNTNAP2, COA7, COASY, COQ2, COQ8A, |
| | COX6A1, CPA6, CPLX1, CPT1C, CRAT, CSF1R, CSNK2B, CSTB, |
| | CTDP1, CTSD, CTSF, CUX2, CWF19L1, CYFIP2, CYP2U1, CYP7B1, |
| | DAB1, DAGLA, DALRD3, DBN1, DCAF8, DCTN1, DCX, DDHD1, |
| | DDHD2, DEAF1, DENND5A, DEPDC5, DGUOK, DHDDS, DHTKD1, |
| | DHX16, DIAPH1, DIAPH3, DLL1, DMXL2, DNAJB2, DNAJC13, |
| | DNAJC3, DNAJC6, DNM1, DNM2, DNMT1, DOCK7, DPP6, DST, |
| | DSTYK, DYNC1H1, ECHS1, EEF1A2, EEF2, EFHC1, EGR2, EIF2S3, |
| | EIF4G1, ELOVL4, ELOVL5, ELP1, ELP3, EMC1, EML1, ENTPD1, |
| | EPM2A, EPRS1, ERBB4, ERLIN1, ERLIN2, EXOSC3, EXOSC8, |
| | EXOSC9, EXT2, FA2H, FAR1, FARS2, FARSB, FAT2, FBLN5, |
| | FBXO38, FBXO7, FDXR, FGD4, FGF12, FGF14, FGGY, FIG4, |
| | FLVCR1, FOLR1, FRRS1L, FTL, FUS, GABBR2, GABRA1, |
| | GABRA2, GABRA5, GABRB1, GABRB2, GABRB3, GABRD, |
| | GABRG2, GAL, GAN, GARS1, GBA, GBA2, GBE1, GDAP1, GDAP2, |
| | GIGYF2, GJB1, GJC2, GLS, GNAO1, GNB4, GOSR2, GOT2, GRIA2, |
| | GRID2, GRIN2A, GRIN2B, GRIN2D, GRM1, GRM7, GRN, GUF1, |
| | HACE1, HARS1, HCN1, HEXA, HEXB, HINT1, HIP1, HK1, |
| | HNRNPA1, HNRNPU, HSD17B10, HSPB1, HSPB3, HSPB8, HSPD1, |
| | HTRA2, HTT, IARS2, IBA57, IER3IP1, IGHMBP2, INF2, IREB2, |
| | IRF2BPL, ITM2B, ITPA, ITPR1, KARS1, KCNA2, KCNB1, KCNC1, |
| | KCNC3, KCND2, KCND3, KCNH1, KCNJ10, KCNK4, KCNMA1, |
| | KCNQ2, KCNQ3, KCNT1, KCNT2, KCTD7, KIDINS220, KIF1A, |
| | KIF1B, KIF1C, KIF5A, KLC2, L1CAM, LAGE3, LGI1, LITAF, |
| | LMNA, LMNB2, LNPK, LRRK2, LRSAM1, MAG, MAPK10, MAPT, |
| | MARCHF6, MARS1, MARS2, MATR3, MCM3AP, MDH1, MDH2, |
| | MED25, MEF2C, MFN2, MFSD8, MME, MORC2, MPV17, MPV17, |
| | MPZ, MT-ATP6, MT-CO1, MT-CO3, MT-CYB, MTHFS, MTMR2, |
| | MT-ND1, MT-ND2, MT-ND4, MT-ND4L, MT-ND5, MT-ND6, |
| | MTOR, MTPAP, MYH14, NACC1, NAGA, NAGLU, NALCN, NAXD, |
| | NAXE, NDRG1, NECAP1, NEFH, NEFL, NEK1, NEUROD2, NGF, |
| | NHLRC1, NHLRC2, NIPA1, NKX6-2, NOP56, NOTCH2NLC, |
| | NPRL2, NPRL3, NRROS, NT5C2, NTRK2, NUP107, NUP133, OPA1, |
| | OPTN, OSGEP, OTOF, OTUD6B, OXR1, P4HTM, PACS2, PAK1, |
| | PANK2, PARK7, PARS2, PCDH12, PCDH19, PCLO, PCNA, PCYT2, |
| | PDK3, PDXK, PDYN, PFN1, PHACTR1, PHF21A, PHF6, PIGA, |
| | PIGB, PIGH, PIGK, PIGN, PIGP, PIGQ, PIGS, PIGT, PIGU, PIK3R5, |
| | PINK1, PLA2G6, PLCB1, PLD3, PLEKHG5, PLP1, PLPBP, PMP2, |
| | PMP22, PMPCA, PMPCB, PNKP, PNPLA6, PNPO, POLG, PPP2R2B, |
| | PPP3CA, PPP5C, PPT1, PRDM12, PRDM12, PRDM8, PRICKLE1, |
| | PRICKLE2, PRICKLE3, PRKCG, PRKN, PRPH, PRPS1, PRRT2, |
| | PRUNE1, PRX, PSAP, PSEN1, PSEN2, PTPN23, PUM1, QARS1, |
| | RAB10, RAB7A, RAPGEF2, RARS2, REEP1, REEP2, RELN, REPS1, |
| | RETREG1, RFC1, RHOBTB2, RNF13, ROGDI, RORA, RORB, RPIA, |
| | RRM2B, RTN2, RUBCN, SACS, SAMD12, SARS1, SBF1, SBF2, |
| | SBF2, SCARB2, SCN11A, SCN1A, SCN1B, SCN2A, SCN3A, SCN8A, |
| | SCN9A, SCYL1, SELENOI, SEMA6B, SEPSECS, SERPINI1, |
| | SETD1A, SETX, SH3TC2, SIGMAR1, SIK1, SIRT2, SLC12A1, |
| | SLC12A5, SLC12A6, SLC13A5, SLC1A2, SLC1A4, SLC25A19, |
| | SLC25A22, SLC25A46, SLC2A1, SLC30A10, SLC33A1, SLC35A2, |
| | SLC35A3, SLC39A14, SLC44A1, SLC45A1, SLC5A6, SLC5A7, |
| | SLC6A1, SLC6A3, SLC9A1, SLC9A6, SMC1A, SMN1, SMN2, |
| | SMPD1, SNAP29, SNCA, SNCAIP, SNIP1, SNX14, SOD1, SORD, |
| | SORL1, SPART, SPAST, SPATA5, SPG11, SPG21, SPG7, SPTAN1, |
| | SPTBN2, SPTBN4, SPTLC1, SPTLC2, SQSTM1, SRPX2, ST3GAL3, |
| | ST3GAL5, STUB1, STX1B, STXBP1, SUMF1, SUOX, SURF1, SYN1, |
| | SYNE1, SYNJ1, SYT14, SZT2, TANGO2, TARDBP, TBC1D24, |
| | TBCD, TBCE, TBK1, TBP, TCF4, TDP1, TDP2, TECPR2, TFG, |
| | TGM6, THG1L, TIMM50, TMEM106B, TMEM175, TMEM230, |
| | TMEM240, TMX2, TNRC6A, TOE1, TP53RK, TPP1, TPRKB, |
| | TRAK1, TRAPPC2L, TRAPPC6B, TREM2, TREX1, TRIM2, TRIP4, |
| | TRPC3, TRPM7, TRPV4, TSC1, TSC2, TSEN2, TSEN34, TSEN54, |
| | TTBK2, TTR, TUBA4A, TUBGCP2, TWNK, TXN2, TYMP, UBA1, |
| | UBA5, UBAP1, UBQLN2, UBQLN4, UBTF, UCHL1, UGDH, UGP2, |
| | UNC5C, UNC80, VAC14, VAMP1, VAPB, VARS1, VCP, VPS13A, |
| | VPS13C, VPS13D, VPS35, VPS37A, VPS53, VRK1, VWA3B, |
| | WARS1, WASF1, WASHC5, WDR4, WDR45, WDR45B, WDR73, |
| | WNK1, WWOX, XRCC1, YARS1, YEATS2, YWHAG, ZEB2, |
| | ZFYVE26, ZFYVE27, ZNHIT3 |
| Cardiovascular | AARS2, ABCC9, ACADVL, ACTC1, ACTN2, AGK, ALPK3, BAG3, |
| | BRAF, CALR3, CAV3, CDH2, CRYAB, CSRP3, CTNNA3, DES, |
| | DMD, DNAJC19, DSC2, DSG2, DSP, DTNA, EMD, EYA4, FKRP, |
| | FKTN, FLNC, GATA4, GATAD1, GJA5, GRM7, GTPBP3, JPH2, JUP, |
| | KRAS, LAMA4, LDB3, LMNA, MAP2K1, MAP2K2, MIB1, MT- |
| | ATP6, MT-ATP8, MT-CYB, MTO1, MYBPC3, MYH6, MYH7, |
| | MYL2, MYL3, MYLK2, MY06, MYOZ2, MYPN, NDUFB11, NEXN, |
| | NPPA, OPA1, PKP2, PLN, PPCS, PRDM16, PRKAG2, PSEN1, |
| | PSEN2, RAF1, RBM20, RYR2, SCN5A, SCO2, SDHA, SGCD, |
| | SLC25A4, TAZ, TBX20, TBX5, TCAP, TGFB3, TMEM43, TMPO, |
| | TNNC1, TNNI3, TNNI3K, TNNT2, TPM1, TSFM, TTN, VCL, ZFPM2 |
| Metabolic | AGL, ALDOA, ARSB, BCKDHA, BCKDHB, DBT, ENO3, EPM2A, |
| | ETFA, ETFB, ETFDH, G6PC, GAA, GALNS, GBA, GBE1, GCDH, |
| | GCH1, GLB1, GNS, GUSB, GYG1, GYS2, HGSNAT, HYAL1, IDS, |
| | IDUA, LAMP2, LDHA, NAGLU, PAH, PFKM, PGAM2, PGM1, |
| | PHKA1, PHKA2, PHKB, PHKG2, PRKAG2, PSAP, PTS, PYGL, |
| | PYGM, QDPR, SGSH, SUGCT, SUMF1, VPS33A |
| | AAGAB, ANAPC1, AQP5, BMS1, BRAF, CAST, CD151, CDH1, |
| | CDH3, COL11A1, COL17A1, COL7A1, CST6, CTNND1, CTSC, |
| | CYP26C1, DDB2, DPH1, DSG1, DSP, DST, EDA, EDAR, |
| | EDARADD, ERCC6, ERCC2, ERCC3, ERCC4, ERCC5, EVC, EVC2, |
| | EXPH5, FGF10, FGFR2, FGFR3, GJA1, GJB2, GJB3, GJB4, GJB6, |
| | GRHL2, HOXC13, IFT122, IFT43, IKBKG, ITGA3, ITGA6, ITGB4, |
| | JUP, KANK2, KDF1, KDSR, KLHL24, KRAS, KREMEN1, KRT1, |
| | KRT14, KRT16, KRT17, KRT5, KRT6A, KRT6B, KRT6C, KRT74, |
| | KRT83, KRT85, KRT9, LAMA3, LAMB3, LAMC2, LORICRIN, |
| | MAP2K1, MAP2K2, MBTPS2, MSX1, NECTIN1, NECTIN4, |
| | NFKBIA, NLRP1, PKP1, PLEC, POLH, POMP, PRKD1, RHBDF2, |
| | RHOA, RIPK4, RSPO1, SASH1, SERPINB7, SLURP1, SMARCAD1, |
| | SNAP29, TAT, TP63, TRPM4, TRPV3, TSPEAR, TWIST2, WDR19, |
| | WDR35, WNT10A, XPA, XPC |
| Mitochondrial | AARS2, ACAD9, AGK, AIFM1, ATP5F1A, ATP5F1D, ATP5F1E, |
| | ATP5MD, ATPAF2, BCS1L, BOLA3, C12orf65, C1QBP, CARS2, |
| | COA3, COA5, COA6, COA8, COQ2, COQ4, COQ6, COQ7, COQ8A, |
| | COQ9, COX10, COX14, COX15, COX20, COX4I1, COX5A, |
| | COX6A2, COX6B1, COX8A, CYC1, DGUOK, DNA2, EARS2, |
| | ELAC2, FARS2, FASTKD2, FBXL4, FDX2, FLAD1, FOXRED1, |
| | GATB, GATC, GFER, GFM1, GFM2, GTPBP3, IBA57, ISCA2, |
| | LRPPRC, LYRM4, LYRM7, MARS2, MGME1, MICOS13, MIPEP, |
| | MPV17, MRM2, MRPL12, MRPL3, MRPL44, MRPS14, MRPS16, |
| | MRPS2, MRPS22, MRPS23, MRPS28, MRPS34, MRPS7, MT-ATP6, |
| | MT-ATP8, MT-CO1, MT-CO2, MT-CO3, MT-CYB, MTFMT, MT- |
| | ND1, MT-ND2, MT-ND3, MT-ND4, MT-ND4L, MT-ND5, MT-ND6, |
| | MTO1, NARS2, NDUFA1, NDUFA10, NDUFA11, NDUFA12, |
| | NDUFA13, NDUFA2, NDUFA4, NDUFA6, NDUFA9, NDUFAF1, |
| | NDUFAF2, NDUFAF3, NDUFAF4, NDUFAF5, NDUFAF6, |
| | NDUFAF8, NDUFB11, NDUFB3, NDUFB8, NDUFB9, NDUFS1, |
| | NDUFS2, NDUFS3, NDUFS4, NDUFS6, NDUFS7, NDUFS8, |
| | NDUFV1, NDUFV2, NFU1, NSUN3, NUBPL, OPA1, PDHA1, PDSS1, |
| | PDSS2, PET100, PET117, PINK1, PNPLA4, PNPT1, POLG, POLG2, |
| | PRICKLE3, PUS1, QRSL1, RMND1, RNASEH1, RRM2B, SCO1, |
| | SCO2, SDHA, SDHAF1, SDHD, SFXN4, SLC25A10, SLC25A21, |
| | SLC25A26, SLC25A4, SUCLA2, SUCLG1, SURF1, TACO1, TARS2, |
| | TFAM, TIMM22, TIMMDC1, TK2, TMEM126B, TMEM70, TOP3A, |
| | TRIT1, TRMT10C, TRMT5, TSFM, TTC19, TUFM, TWNK, TXN2, |
| | TYMP, UQCC2, UQCC3, UQCRB, UQCRC2, UQCRFS1, UQCRQ, |
| | VARS2, WARS2, YARS2 |
| Muscle | ANO5, B3GALNT2, B4GAT1, BVES, CAPN3, CAV3, CHKB, |
| | COL12A1, COL6A1, COL6A2, COL6A3, CRPPA, DAG1, DES, |
| | DNAJB6, DPM1, DPM2, DPM3, DYSF, EMD, FHL1, FKRP, FKTN, |
| | GIPC1, GMPPB, HNRNPDL, INPP5K, ITGA7, LAMA2, LARGE1, |
| | LIMS2, LMNA, MYOT, PLEC, POGLUT1, POMGNT1, POMGNT2, |
| | POMK, POMT1, POMT2, POPDC3, PYROXD1, RXYLT1, SGCA, |
| | SGCB, SGCD, SGCG, SYNE1, SYNE2, TCAP, TMEM43, TNPO3, |
| | TOR1AIP1, TRAPPC11, TRIM32, TRIP4, TTN |
| Liver | ABCB11, ABCB4, AKR1D1, AMACR, ATP8B1, CYP7B1, HSD3B7, |
| | NR1H4, SLC25A13, TJP2 |
| Hearing | ABCC1, ABHD12, ABHD5, ACOX1, ACSL4, ACTB, ACTG1, |
| | ADAMTS17, ADCY1, ADGRV1, AIFM1, ALMS1, AMMECR1, |
| | ANKH, AP000812.4, AP1B1, AP1S1, ARSG, ATP1A3, ATP6V1B1, |
| | ATP6V1B2, BCAP31, BCS1L, BDP1, BRAF, BSND, CABP2, |
| | CACNA1D, CCDC50, CD151, CD164, CDC14A, CDH11, CDH23, |
| | CEACAM16, CEP250, CEP78, CIB2, CISD2, CLCNKA, CLCNKB, |
| | CLDN14, CLIC5, CLPP, CLRN1, COCH, COL11A1, COL11A2, |
| | COL2A1, COL4A3, COL4A4, COL4A5, COL4A6, COL9A1, COL9A2, |
| | COQ6, CRYM, DCAF17, DCDC2, DCHS1, DIABLO, DIAPH1, |
| | DIAPH3, DLX5, DMXL2, DNAJC3, DNMT1, DSPP, EDN3, EDNRB, |
| | ELMOD3, EPS8, EPS8L2, ERAL1, ERCC2, ERCC3, ERCC5, ERCC6, |
| | ERCC8, ESPN, ESRP1, ESRRB, EXOSC2, EYA1, EYA4, FAT4, |
| | FDXR, FGF9, FGFR3, FITM2, FKBP14, FLNA, FOXC1, GAB1, |
| | GATA3, GFER, GIPC3, GJB2, GJB3, GJB6, GPC4, GPRASP2, |
| | GPSM2, GRAP, GRHL2, GRXCR1, GRXCR2, GSDME, HARS1, |
| | HARS2, HGF, HOMER2, HSD17B4, IARS2, IGF1, ILDR1, ITM2B, |
| | JAG1, KARS1, KCNE1, KCNE5, KCNH2, KCNJ10, KCNQ1, KCNQ4, |
| | KITLG, KRAS, LARS2, LHFPL5, LMX1A, LONP1, LOXHD1, |
| | LOXL3, MAF, MARS2, MARVELD2, MCM2, MET, MGP, MITF, |
| | MPZ, MPZL2, MSRB3, MT-CO1, MT-CYB, MYH14, MYH9, |
| | MYO15A, MYO1F, MYO3A, MY06, MYO7A, NARS2, NDP, NF2, |
| | NLRP3, NOG, OPA1, OSBPL2, OTOA, OTOF, OTOG, OTOGL, |
| | P2RX2, PAX1, PAX3, PCDH15, PCNA, PDE1C, PDZD7, PEX1, |
| | PEX6, PEX7, PHYH, PIGL, PISD, PJVK, PLS1, PMP22, PNPT1, |
| | POU4F3, PPIP5K2, PPP2R3C, PRPS1, PTPN11, RAB40AL, RAF1, |
| | RDX, REST, RIPOR2, ROR1, RPGR, RPS23, S1PR2, SALL1, |
| | SERAC1, SERPINB6, SIX1, SIX5, SLC17A8, SLC19A2, SLC26A4, |
| | SLC26A5, SLC33A1, SLC44A4, SLC4A11, SLC52A2, SLC52A3, |
| | SLC9A1, SLITRK6, SMPX, SNAI2, SOX10, SPATA5, SPNS2, |
| | SPTBN4, STRC, SYNE4, TBC1D24, TBL1X, TBL1Y, TBX22, |
| | TECTA, THRB, TIMM8A, TMC1, TMEM132E, TMEM67, TMIE, |
| | TMPRSS3, TNC, TNFRSF11A, TPRN, TRAPPC12, TRIOBP, TRMU, |
| | TRRAP, TSPEAR, TUBB4B, TWNK, TXNL4A, TYR, USH1C, |
| | USH1G, USH2A, WBP2, WFS1, WHRN |
| Opthalmic | ARMS2, ABCA4, ABCA4, ABHD12, ABHD5, ADGRV1, AGBL1, |
| | AGBL5, AGK, AGPS, AHR, AIPL1, ARHGEF18, ARL6, ARSG, |
| | ASB10, BBS2, BEST1, BFSP1, BFSP2, C2, C3, C8orf37, C9, CA4, |
| | CACNA1F, CDH23, CEP290, CERKL, CFB, CFH, CFI, CHMP4B, |
| | CHST6, CIB2, CLCC1, CLPB, CLRN1, CNGA1, CNGA3, CNGB1, |
| | CNGB3, COL11A1, COL18A1, COL2A1, COL8A2, CRB1, CRB2, |
| | CRX, CRYAA, CRYAA2, CRYAB, CRYBA1, CRYBA2, CRYBA4, |
| | CRYBB1, CRYBB2, CRYBB3, CRYGB, CRYGC, CRYGD, CRYGS, |
| | CST3, CTDP1, CWC27, CX3CR1, CYP1B1, CYP27A1, CYP4V2, |
| | DCN, DHDDS, DHX38, DMPK, DNMBP, EBP, ELOVL4, EPG5, |
| | EPHA2, ERCC1, ERCC2, ERCC6, ESPN, EXOSC2, EYS, FAM126A, |
| | FAM161A, FAR1, FBLN5, FLVCR1, FOXE3, FSCN2, FTL, FYCO1, |
| | GALK1, GALT, GCNT2, GDF6, GFER, GJA1, GJA3, GJA8, GLS, |
| | GNAT1, GNB3, GNPAT, GPATCH3, GRHL2, GRK1, GRM6, GSN, |
| | GUCA1B, GUCY2D, HARS1, HGSNAT, HK1, HMCN1, HMX1, |
| | HSF4, HTRA1, IARS2, IDH3B, IFT140, IFT172, IFT43, IMPDH1, |
| | IMPG2, INPP5K, IQCB1, KCNA4, KCNJ13, KIAA1549, KIF3B, KIZ, |
| | KLHL7, KRT12, KRT3, LCA5, LCAT, LEMD2, LIM2, LONP1, |
| | LOXL1, LRAT, LRIT3, LSS, LTBP2, MAF, MAK, MBNL1, MERTK, |
| | MFRP, MIP, MSMO1, MT-ATP6, MT-CO1, MT-CO3, MT-CYB, MT- |
| | ND1, MT-ND2, MT-ND4, MT-ND4L, MT-ND5, MT-ND6, MVK, |
| | MYH9, MYO7A, MYOC, NEK2, NHS, NMNAT1, NPHP1, NPHP4, |
| | NR2E3, NRL, NTF4, NUP188, NYX, OCRL, OFD1, OPA3, OPTN, |
| | OVOL2, PCARE, PCDH15, PDE6A, PDE6B, PDE6G, PDZD7, PEX7, |
| | PHYH, PIK3C2A, PIKFYVE, PISD, PITX3, PNPLA6, POMGNT1, |
| | PRCD, PRICKLE3, PROM1, PRPF3, PRPF31, PRPF4, PRPF6, PRPF8, |
| | PRPH2, PRPS1, RAB3GAP2, RAX2, RBP3, RD3, RDH11, RDH12, |
| | RECQL4, REEP6, RGR, RHO, ROM1, RP1, RP1L1, RP2, RP9, RPE65, |
| | RPGR, RPGRIP1, SAG, SCAPER, SDCCAG8, SEMA4A, SIPA1L3, |
| | SIX6, SLC16A12, SLC24A1, SLC2A1, SLC33A1, SLC4A11, SLC4A4, |
| | SLC7A14, SNRNP200, SPATA7, SRD5A3, TACSTD2, TBK1, TCF4, |
| | TDRD7, TEK, TGFBI, TKFC, TLR4, TMCO1, TMEM67, TOPORS, |
| | TRAF3IP1, TRNT1, TRPM1, TTC8, TUBB4B, TULP1, UBIAD1, |
| | UNC45B, USH1C, USH1G, USH2A, USP45, VCAN, VIM, VSX1, |
| | VSX2, WDR19, WDR36, WFS1, WHRN, ZEB1, ZNF408, ZNF513 |
| Cancer | A2M, AARS2, ABCB1, ABCC1, ABCC2, ABCC3, ABCC5, ABCC6, |
| | ABCG2, ABI1, ABL1, ABL2, ACAP1, ACKR3, ACLY, ACO1, ACP3, |
| | ACSL3, ACVR1, ACVR1B, ACVR2A, ACVR2B, ADAM10, ADAM9, |
| | ADAMTS1, ADAMTS14, ADAMTS18, ADAMTS20, ADAMTS3, |
| | ADAMTS4, ADAMTS5, ADAMTS6, ADAMTS8, ADAMTS9, |
| | ADCY1, ADGRB1, ADM, ADNP, ADORA2A, ADRA1B, AFDN, |
| | AFF1, AFF3, AFF4, AFP, AGER, AHNAK2, AHR, AHSG, AJUBA, |
| | AK9, AKAP12, AKAP9, AKR1B1, AKT1, AKT2, AKT3, ALB, |
| | ALCAM, ALDOA, ALDOB, ALDOC, ALK, ALKBH6, ALPK2, |
| | ALPL, ALPP, AMER1, AMPH, ANAPC1, ANG, ANGPT1, ANGPT2, |
| | ANK3, ANKRD12, ANXA1, ANXA11, ANXA2, ANXA4, ANXA7, |
| | AOC3, AP2B1, APAF1, APC, APEX1, APOA1, APOA2, APOBEC3B, |
| | APOC1, APOC3, APOD, APOE, APOL2, APPBP2, AR, AREG, ARG2, |
| | ARHGAP26, ARHGAP32, ARHGAP35, ARHGEF12, ARHGEF6, |
| | ARID1A, ARID1B, ARID2, ARID5B, ARNT, ASPH, ASPM, |
| | ASPSCR1, ASXL1, ASXL2, ATF1, ATG13, ATIC, ATM, ATOH1, |
| | ATP1A1, ATP2B3, ATP7B, ATR, ATRX, AURKA, AURKB, AXIN1, |
| | AXIN2, AZGP1, B2M, BAD, BAG1, BAP1, BARD1, BAX, BCL10, |
| | BCL11A, BCL11B, BCL2, BCL2A1, BCL2L1, BCL2L2, BCL2L2- |
| | PABPN1, BCL3, BCL6, BCL7A, BCL9, BCL9L, BCLAF1, BCOR, |
| | BCORL1, BCR, BDNF, BHMT2, BIRC2, BIRC3, BIRC5, BIRC6, |
| | BIVM-ERCC5, BLK, BLM, BLMH, BMI1, BMP2, BMP4, BMPR1A, |
| | BNIP3, BNIP3L, BRAF, BRCA1, BRCA2, BRD3, BRD4, BRIP1, |
| | BRMS1, BTG1, BTG2, BTK, BUB1B, C1QBP, C3orf70, C6, C7, CA8, |
| | CACNA1D, CAD, CALCA, CALR, CAMTA1, CANT1, CANX, CAP2, |
| | CAPN6, CARD11, CARM1, CARS1, CASC3, CASP1, CASP10, |
| | CASP2, CASP3, CASP4, CASP5, CASP6, CASP7, CASP8, CASP9, |
| | CAST, CAT, CAV1, CBFA2T3, CBFB, CBL, CBLB, CBLC, CCAR1, |
| | CCDC120, CCDC6, CCKBR, CCL11, CCL13, CCL14, CCL16, CCL18, |
| | CCL19, CCL2, CCL21, CCL23, CCL3, CCL4, CCL5, CCL7, CCL8, |
| | CCN2, CCN4, CCNA1, CCNA2, CCNB1, CCNB1IP1, CCNB2, |
| | CCND1, CCND2, CCND3, CCNE1, CCNE2, CCNG1, CCNG2, CCNH, |
| | CCR10, CCR7, CD14, CD1D, CD24, CD27, CD274, CD36, CD38, |
| | CD40, CD40LG, CD44, CD46, CD52, CD59, CD70, CD74, CD79A, |
| | CD79B, CD82, CD9, CDC16, CDC20, CDC25A, CDC25B, CDC25C, |
| | CDC27, CDC34, CDC37, CDC6, CDC73, CDH1, CDH11, CDH17, |
| | CDH5, CDK1, CDK12, CDK2, CDK4, CDK6, CDK7, CDKN1A, |
| | CDKN1B, CDKN1C, CDKN2A, CDKN2C, CDX2, CEACAM5, |
| | CEACAM6, CEBPA, CENPF, CEP43, CEP76, CFH, CFHR1, CFLAR, |
| | CFTR, CGA, CHCHD7, CHD4, CHD7, CHD8, CHEK1, CHEK2, |
| | CHFR, CHGA, CHI3L1, CHP2, CIB2, CIC, CIITA, CKB, CKS1B, |
| | CKS2, CLDN3, CLDN4, CLDN7, CLEC3B, CLIC1, CLIP1, CLSTN1, |
| | CLTC, CLTCL1, CLU, CNBD1, CNBP, CNKSR1, CNN1, CNOT3, |
| | CNTF, CNTRL, COL11A1, COL17A1, COL18A1, COL1A1, COL1A2, |
| | COL2A1, COL4A2, COL4A3, COL4A4, COL4A5, COL5A1, COL5A3, |
| | COL6A1, COX17, CP, CRABP1, CRADD, CREB1, CREB3L1, |
| | CREB3L2, CREBBP, CRLF2, CRP, CRTC1, CRTC3, CRYAB, |
| | CSDE1, CSE1L, CSF1, CSF1R, CSF2, CSF2RA, CSF3, CSF3R, |
| | CSN1S1, CSNK1E, CSNK2A2, CSNK2B, CST3, CST6, CSTA, CSTB, |
| | CTAG1A, CTAG1B, CTAG2, CTCF, CTNNB1, CTNNBL1, CTNND1, |
| | CTSB, CTSD, CTSH, CTSL, CTTN, CUL1, CUL2, CUL4B, CUL5, |
| | CUX1, CXCL1, CXCL10, CXCL13, CXCL2, CXCL5, CXCL8, |
| | CXCL9, CXCR1, CXCR2, CXCR4, CYB5R3, CYLD, CYP19A1, |
| | CYP1A2, CYP2C19, CYP2E1, CYP3A4, CYP3A5, DAD1, DAPK1, |
| | DAXX, DBI, DCC, DCN, DCTN1, DDB2, DDIT3, DDR2, DDX10, |
| | DDX3X, DDX5, DDX6, DEFA1, DEFA1B, DEFA3, DEK, DES, |
| | DHFR, DHX9, DIAPH1, DIAPH3, DICER1, DIS3, DLC1, DMD, |
| | DNAH12, DNAJB1, DNAJC2, DNER, DNM2, DNMT3A, DOCK2, |
| | DROSHA, DST, DUSP1, DUSP14, DUSP4, DVL3, DYNLL1, DYRK2, |
| | E2F1, E2F3, E2F5, EBAG9, EBF1, EDN1, EEF1A1, EEF2, EFNA1, |
| | EFNA2, EFNA5, EFNB1, EFNB2, EFNB3, EGF, EGFR, EGR1, EI24, |
| | EIF2S2, EIF3E, EIF3H, EIF4A2, EIF4E, EIF4EBP1, EIF4G1, EIF4H, |
| | EIF5A, ELANE, ELF3, ELF4, ELK3, ELK4, ELL, EML4, ENC1, ENG, |
| | ENO1, ENO2, ENPP2, EP300, EPAS1, EPCAM, EPHA1, EPHA2, |
| | EPHA3, EPHA4, EPHA7, EPHA8, EPHB2, EPHB3, EPHB4, EPHX1, |
| | EPO, EPOR, EPS15, ERBB2, ERBB3, ERBB4, ERC1, ERCC1, |
| | ERCC2, ERCC3, ERCC4, ERCC5, ERCC6, ERG, ESR1, ESR2, |
| | ETNK1, ETV1, ETV4, ETV5, ETV6, EWSR1, EXT1, EXT2, EZH1, |
| | EZH2, EZR, F13A1, F13B, F2, F3, FABP1, FABP2, FABP4, FABP5, |
| | FADD, FAF1, FAM166A, FANCA, FANCC, FANCD2, FANCE, |
| | FANCF, FANCG, FAP, FAS, FASLG, FASN, FAT1, FAT4, FBN2, |
| | FBXO11, FBXO6, FBXW7, FCER2, FCGR2B, FCRL4, FEN1, FES, |
| | FEV, FGA, FGB, FGF1, FGF17, FGF18, FGF19, FGF2, FGF23, FGF3, |
| | FGF4, FGF6, FGF7, FGF8, FGF9, FGFBP1, FGFR1, FGFR2, FGFR3, |
| | FGFR4, FGG, FH, FHIT, FIP1L1, FKBP5, FKBP8, FLCN, FLG, FLI1, |
| | FLT1, FLT3, FLT4, FMO5, FN1, FOLH1, FOS, FOSL1, FOXA1, |
| | FOXA2, FOXJ1, FOXL2, FOXM1, FOXO1, FOXO3, FOXO4, FOXP1, |
| | FOXQ1, FRMD7, FSCN1, FSHB, FST, FSTL3, FTH1, FTL, FUBP1, |
| | FUS, FZD1, FZD2, G6PD, GADD45A, GADD45G, GAS1, GAS7, |
| | GAST, GATA1, GATA2, GATA3, GCLM, GDF15, GDNF, GH1, GH2, |
| | GJA1, GJB5, GLO1, GMNN, GNA11, GNA13, GNAI1, GNAQ, |
| | GNAS, GNB1, GNPTAB, GOLGA5, GOPC, GOT1, GOT2, GPA33, |
| | GPC3, GPHN, GPI, GPS2, GPX1, GPX2, GRB10, GRB2, GRB7, |
| | GRIN2A, GSK3A, GSN, GSR, GSTM1, GSTM3, GSTP1, GTF2H1, |
| | GUSB, H2AC6, H3-3A, H3-3B, H3C2, H4C9, HDAC10, HDAC2, |
| | HDAC5, HERPUD1, HEY1, HGF, HGFAC, HIF1A, HIP1, HIP1R, |
| | HK1, HK2, HLA-A, HLA-B, HLA-G, HLF, HMGA1, HMGA2, |
| | HMGXB4, HMOX1, HNF1A, HNRNPA2B1, HOOK3, HOXA11, |
| | HOXA13, HOXA5, HOXA9, HOXC11, HOXC13, HOXD11, |
| | HOXD13, HP, HPGD, HPN, HRAS, HSF1, HSP90AA1, HSP90AB1, |
| | HSP90B1, HSPA1L, HSPA2, HSPA4, HSPA8, HSPB1, HSPD1, |
| | HSPE1, HSPH1, HUWE1, IBSP, ICAM1, ID1, ID2, ID3, IDH1, IDH2, |
| | IDO1, IFNA1, IFNA13, IFNAR1, IFNAR2, IFNB1, IFNG, IGF1R, |
| | IGF2, IGF2R, IGFBP2, IGFBP3, IKBKB, IKZF1, IL10, IL11, IL12A, |
| | IL13, IL13RA2, IL15, IL16, IL17A, IL17B, IL18, IL1A, IL1B, IL1R1, |
| | IL1R2, IL1RN, IL2, IL21R, IL24, IL2RA, IL2RB, IL2RG, IL4, IL4R, |
| | IL5, IL6, IL6R, IL6ST, IL7, IL7R, IL9, ILF3, ILK, ING1, INHBA, |
| | INHBB, INPPL1, INS, INTS12, IPO7, IRF1, IRF4, IRF6, IRS2, IRS4, |
| | ITGA1, ITGA2, ITGA2B, ITGA3, ITGA4, ITGA5, ITGA6, ITGAM, |
| | ITGAV, ITGB1, ITGB3, ITGB4, ITGB5, ITGB6, ITGB7, ITGB8, |
| | ITIH4, ITK, ITPKB, JAK1, JAK2, JAK3, JKAMP, JTB, JUN, JUND, |
| | JUP, KALRN, KAT2B, KAT6A, KAT6B, KCNJ5, KDM5A, KDM5C, |
| | KDM6A, KDR, KDSR, KEAP1, KEL, KIAA1109, KIF2A, KIF2C, |
| | KIF5B, KIFC3, KISS1, KIT, KITLG, KLF4, KLF5, KLF6, KLHL8, |
| | KLK10, KLK11, KLK13, KLK14, KLK15, KLK2, KLK3, KLK4, |
| | KLK5, KLK6, KLK7, KLK8, KLK9, KLRK1, KMT2A, KMT2B, |
| | KMT2C, KMT2D, KNL1, KRAS, KRT13, KRT14, KRT15, KRT17, |
| | KRT18, KRT19, KRT4, KRT8, KTN1, LALBA, LAMB1, LAMC1, |
| | LASP1, LATS1, LATS2, LCK, LCN1, LCP1, LCTL, LDHA, LEF1, |
| | LEP, LEPR, LGALS3, LGALS3BP, LGALS4, LGI1, LGMN, LHB, |
| | LHX1, LIF, LIFR, LIG4, LIMK1, LMNA, LMO1, LMO2, LPP, LRIG3, |
| | LRP1B, LRP6, LRRK2, LTA, LTA4H, LTB, LTBR, LTF, LUM, LYL1, |
| | LYN, LZTR1, MAD2L1, MAD2L2, MAF, MAFB, MAGEA3, |
| | MAGEA4, MAGEA6, MAGEB5, MAGEB6, MAGEC1, MAGEC2, |
| | MAGEC3, MAGED1, MAGED2, MAGI1, MALT1, MAML2, |
| | MAP2K1, MAP2K2, MAP2K4, MAP3K1, MAP3K13, MAP3K3, |
| | MAP3K4, MAP4K3, MAPK1, MAPK14, MAPK3, MAPK7, MAPK8, |
| | MAPK8IP1, MAPKAPK2, MAST2, MATK, MAX, MBD1, MBD2, |
| | MBD4, MCL1, MCM2, MCM3, MCM5, MCM7, MDC1, MDH1, |
| | MDK, MDM2, MDM4, MECOM, MECP2, MED1, MED12, MED13, |
| | MED17, MED23, MEF2A, MEN1, MET, METTL14, MFGE8, MGA, |
| | MGMT, MIA, MIF, MITF, MKI67, MLF1, MLH1, MLH3, MLLT1, |
| | MLLT10, MLLT11, MLLT3, MLLT6, MME, MMP1, MMP10, |
| | MMP11, MMP12, MMP13, MMP14, MMP15, MMP16, MMP2, |
| | MMP3, MMP7, MMP8, MMP9, MN1, MORC4, MPL, MPO, MRE11, |
| | MRTFA, MSH2, MSH6, MSI2, MSLN, MSMB, MSN, MSR1, MST1, |
| | MT1A, MT1G, MTA1, MTCP1, MTOR, MUC1, MUC17, MUTYH, |
| | MVP, MXI1, MXRA5, MYB, MYBL2, MYC, MYCL, MYCN, |
| | MYD88, MYH11, MYH9, MYO5A, MYOCD, MYOD1, MYOG, |
| | NAB2, NAGA, NAIP, NAMPT, NAT2, NAV3, NBN, NBPF1, |
| | NBPF10, NCAM1, NCOA1, NCOA2, NCOA3, NCOA4, NCOR1, |
| | NCOR2, NDRG1, NEB, NEDD4L, NEDD8, NEO1, NF1, NF2, |
| | NFATC2, NFE2L2, NFIB, NFKB1, NFKB2, NFKBIA, NFKBIE, NGF, |
| | NGFR, NIBAN1, NIN, NKX2-1, NKX3-1, NLRP3, NME1, NME2, |
| | NONO, NOS1, NOS2, NOS3, NOTCH1, NOTCH2, NOTCH3, NPM1, |
| | NQO1, NR0B1, NR1H2, NR4A2, NR4A3, NRAS, NRG1, NRG2, |
| | NRG3, NRP1, NRP2, NSD1, NSD2, NSD3, NT5C2, NTF3, NTF4, |
| | NTHL1, NTN1, NTN4, NTRK1, NTRK2, NTRK3, NUCB2, NUDT1, |
| | NUMA1, NUMB, NUP210L, NUP214, NUP93, NUP98, NUTM1, |
| | NUTM2B, NUTM2D, ODAM, OGG1, OLIG2, OMA1, OR4A16, |
| | OR51E2, OR52N1, ORM1, OSM, OTUD7A, P2RY8, PABPC1, |
| | PAFAH1B2, PAGE4, PALB2, PAPPA, PARP1, PARVB, PATZ1, |
| | PAX3, PAX5, PAX7, PAX8, PBRM1, PBX1, PCBP1, PCM1, PCNA, |
| | PDAP1, PDCD1LG2, PDCD2L, PDE4DIP, PDGFA, PDGFB, |
| | PDGFRA, PDGFRB, PDSS2, PDZD4, PECAM1, PER1, PF4, PGC, |
| | PGF, PGR, PHF20, PHF6, PHLDA1, PHOX2B, PICALM, PIGR, |
| | PIK3CA, PIK3CB, PIK3CG, PIK3R1, PIK3R2, PIK3R3, PIM1, PIM2, |
| | PIM3, PIN1, PIP4K2B, PIP5K1A, PKM, PLAG1, PLAT, PLAU, |
| | PLAUR, PLCG1, PLCG2, PLEC, PLG, PLK1, PLP1, PMEPA1, PML, |
| | PMP22, PMS1, PMS2, PNMT, POLD1, POLE, POLQ, POMC, PON1, |
| | POSTN, POT1, POTEF, POU2AF1, POU2F2, POU5F1, PPA2, PPARG, |
| | PPARGC1A, PPFIBP1, PPM1D, PPP1R15A, PPP2R1A, PPP6C, PPY, |
| | PRCC, PRDM1, PRDM13, PRDM16, PRDX2, PRDX4, PREX2, PRF1, |
| | PRKACA, PRKAR1A, PRKCA, PRKCB, PRKCD, PRKCE, PRKCH, |
| | PRKCI, PRKCQ, PRKDC, PRL, PROC, PRPF8, PRRX1, PRSS1, |
| | PSCA, PSEN1, PSIP1, PSMD4, PTCH1, PTCH2, PTEN, PTGS1, |
| | PTGS2, PTH, PTHLH, PTK2, PTK6, PTN, PTPN11, PTPN13, PTPRB, |
| | PTPRC, PTPRK, PTPRO, PTPRT, PTTG1, PURA, PZP, QKI, |
| | RAB11FIP3, RAB18, RAB25, RAB40A, RABEP1, RAC1, RAD21, |
| | RAD23A, RAD23B, RAD51, RAD51B, RAD51D, RAD52, RAD54B, |
| | RAF1, RANBP2, RANBP3, RAP1GDS1, RARA, RARB, RARG, |
| | RASA1, RB1, RBBP4, RBL1, RBL2, RBM10, RBM15, RBM6, RBMX, |
| | RBP4, RECQL4, REL, RELA, RELB, RET, RHEB, RHOA, RHOB, |
| | RHOC, RHOH, RIT1, RMC1, RMI2, RNF213, RNF43, ROS1, RPA2, |
| | RPGR, RPL10, RPL22, RPL27, RPL5, RPN1, RPS15, RPS2, RPS3, |
| | RPS6KA1, RPS6KA3, RSBN1L, RSPO2, RSPO3, RUNX1, RUNX1T1, |
| | RXRA, RXRB, RXRG, S100A1, S100A2, S100A4, S100A6, S100A7, |
| | S100A8, S100A9, S100B, S1PR1, SACS, SALL4, SART1, SBDS, |
| | SCGB1A1, SCGB1D2, SCGB2A1, SCGB2A2, SDC1, SDC4, SDHA, |
| | SDHAF2, SDHB, SDHC, SDHD, SELE, SELL, SELP, SEMA3B, |
| | SEPTIN2, SERPINA1, SERPINA3, SERPINA5, SERPINB13, |
| | SERPINB2, SERPINB3, SERPINB4, SERPINE1, SERPINF1, SET, |
| | SETBP1, SETD2, SETDB1, SF3B1, SFN, SFPQ, SFRP4, SGK1, |
| | SH2B3, SH3GL1, SHBG, SIN3A, SIRT2, SIRT4, SIX1, SKP2, |
| | SLC19A1, SLC1A3, SLC26A3, SLC2A1, SLC34A2, SLC3A2, |
| | SLC44A3, SLC45A3, SLC4A5, SLPI, SMAD1, SMAD2, SMAD3, |
| | SMAD4, SMARCA4, SMARCB1, SMARCD1, SMARCE1, SMC1A, |
| | SMC3, SMO, SMYD3, SND1, SNX25, SOCS1, SOD1, SOD2, SOS1, |
| | SOX1, SOX17, SOX2, SOX9, SP1, SPARC, SPARCL1, SPATA6, |
| | SPEN, SPINK1, SPINT1, SPINT2, SPOP, SPP1, SPRR1B, SPRR3, |
| | SPRY1, SRC, SRD5A1, SRD5A2, SRSF2, SRSF3, SS18, SS18L1, SST, |
| | SSX1, SSX2, SSX2B, SSX4, ST14, STAG2, STARD3, STAT3, STAT4, |
| | STAT5A, STAT5B, STAT6, STEAP1, STIL, STK11, STK19, STMN1, |
| | STRAP, STRN, STT3A, STX2, SUFU, SULT1E1, SUZ12, SYK, |
| | SYNE1, TAF1, TAF15, TAGLN, TAL1, TAL2, TAP1, TBC1D12, |
| | TBL1XR1, TBX3, TCEA1, TCF12, TCF3, TCF7, TCF7L2, TCL1A, |
| | TCP11L2, TDRD10, TDRD6, TEK, TENT5C, TERT, TET1, TET2, TF, |
| | TFAP2B, TFDP1, TFDP2, TFE3, TFEB, TFF1, TFF2, TFF3, TFG, |
| | TFRC, TG, TGFA, TGFB2, TGFB3, TGFBR2, TGFBR3, TGIF1, |
| | TGM4, TGM7, THBS1, THBS2, THBS4, THPO, THRA, THRB, TIE1, |
| | TIMM17A, TIMP1, TIMP2, TIMP3, TJP2, TK1, TLX1, TLX3, |
| | TMEM127, TMF1, TMPRSS2, TMPRSS3, TNC, TNF, TNFAIP2, |
| | TNFAIP3, TNFRSF10A, TNFRSF10B, TNFRSF10C, TNFRSF10D, |
| | TNFRSF11B, TNFRSF12A, TNFRSF14, TNFRSF17, TNFRSF1A, |
| | TNFRSF1B, TNFRSF4, TNFRSF8, TNFRSF9, TNFSF10, TNFSF11, |
| | TNFSF13, TNFSF13B, TNFSF4, TNFSF8, TNK2, TOM1, TOP1, |
| | TOP2A, TOP3A, TP53, TP53BP1, TP53BP2, TP63, TPD52, TPI1, |
| | TPM1, TPM2, TPM3, TPM4, TPR, TPX2, TRAF1, TRAF2, TRAF3, |
| | TRAF4, TRAF7, TRIM23, TRIM24, TRIM25, TRIM27, TRIM33, |
| | TRIM7, TRIO, TRIP11, TRIP4, TRO, TRRAP, TSC1, TSC2, TSG101, |
| | TSHR, TSPAN8, TSPO, TTLL9, TTR, TUBA1A, TUSC2, TWIST1, |
| | TXLNA, TXNDC8, TXNIP, TXNRD1, TYMP, TYMS, TYRO3, |
| | U2AF1, UBA1, UBE2C, UBE2I, UBE2N, UBR5, UGT1A1, UGT1A10, |
| | UGT1A3, UGT1A4, UGT1A9, UHRF1BP1L, USH1C, USP6, USP8, |
| | VAMP3, VCAM1, VEGFA, VEGFB, VEGFC, VEGFD, VHL, VIL1, |
| | VIP, VTN, VWF, WAS, WASF3, WDCP, WEE1, WFDC2, WIF1, |
| | WNT1, WNT2, WRN, WT1, WWTR1, XBP1, XIAP, XIRP2, XPA, |
| | XPC, XPO1, XRCC1, XRCC2, XRCC3, XRCC4, XRCC5, XRCC6, |
| | YBX1, YWHAB, YWHAE, YWHAH, ZBTB16, ZFHX3, ZFP36L1, |
| | ZFP36L2, ZMYM2, ZNF132, ZNF180, ZNF300, ZNF331, ZNF384, |
| | ZNF471, ZNF483, ZNF521, ZNF620, ZNF750, ZNF814, ZNF844, |
| | ZNF91, ZRANB3, ZRSR2 |
| Genetic | AGPAT2, AGRN, AIPL1, ALG1, ALG11, ALG12, ALG13, ALG14, |
| | ALG2, ALG3, ALG6, ALG8, ALG9, ATP6AP1, ATP6AP2, |
| | B3GALNT2, B3GLCT, B4GALT1, B4GAT1, BPGM, BSCL2, |
| | CACNA1F, CAD, CAV1, CAVIN1, CCDC115, CDAN1, CDIN1, |
| | CEP290, CHAT, CHKB, CHRNA1, CHRNB1, CHRND, CHRNE, |
| | CNGA3, COG1, COG2, COG5, COG6, COG7, COG8, COL12A1, |
| | COL13A1, COL6A1, COL6A2, COL6A3, COLQ, CRB1, CRPPA, |
| | CRX, CYP11B1, CYP17A1, CYP21A2, DAG1, DDOST, DOK7, |
| | DOLK, DPAGT1, DPM1, DPM2, DPM3, DUOX2, DUOXA2, EGLN1, |
| | EPAS1, EPO, EPOR, FCSK, FKRP, FKTN, FOXE1, FUT8, GALNT2, |
| | GDF6, GFPT1, GIPC1, GLIS3, GMPPB, GNAT1, GNB3, GRK1, |
| | GRM6, GUCY2D, HBB, HSD3B2, IGSF1, IMPDH1, INPP5K, IQCB1, |
| | IRS4, ITGA7, IYD, KCNJ13, KCNJ6, KLF1, LAMA2, LARGE1, |
| | LCA5, LMNA, LPIN2, LRAT, LRIT3, LRP4, MAGT1, MGAT2, |
| | MPDU1, MPI, MUSK, MYO7A, MYO9A, NKX2-1, NMNAT1, |
| | NPHP1, NPHP4, NUS1, NKX2-5, NYX, PAX8, PDE6B, PGM1, |
| | PMM2, POMGNT1, POMGNT2, POMK, POMT1, POMT2, POR, |
| | PREPL, RAPSN, RD3, RDH12, RFT1, RHO, RPE65, RPGRIP1, |
| | RXYLT1, SAG, SCN4A, SDCCAG8, SEC23B, SLC18A3, SLC24A1, |
| | SLC25A1, SLC35A1, SLC35A2, SLC35C1, SLC39A8, SLC5A5, |
| | SLC5A7, SNAP25, SPATA7, SRD5A3, SSR4, STAR, STT3A, STT3B, |
| | SYT2, TBL1X, TG, THRA, TMEM165, TMEM199, TPO, TRAF3IP1, |
| | TRHR, TRIP4, TRPM1, TSHR, TUBB4B, TULP1, USP45, VAMP1, |
| | VHL, WDR19 |

### Kits

The disclosure provides kits or articles of manufacture comprising polynucleotides of the disclosure and a preparation for delivery of the polynucleotides to cells. The polynucleotides may be provided as part of a vector of the disclosure. In some embodiments, the kit or article of manufacture further comprises instructions for using the set of the polynucleotides to transform cells to express a gene of interest to produce a polypeptide of interest.

In some cases, the kits may also include a small molecule of the disclosure.

### EXAMPLES

### Methods

NS3a-based regulated CARs were evaluated using lentiviral transduction of T cells and flow cytometry analysis. The CARs evaluated are specific for the ROR1 antigen expressed on the target cell line Jeko1. The Jurkat and Jeko1 cell lines were obtained from American Type Culture Collection (Manassas, VA) and maintained in RPMI 1640 media with Glutamax (Gibco) containing 10% fetal calf serum (Gibco). For lentiviral transduction, Jurkat cells were incubated with lentivirus in complete media plus LentiBOOST at the manufacturers recommended concentration (Sirion Biotech). Eighteen hours after transfection, lentivirus and LentiBOOST were diluted by addition of 1 volume fresh media.

Pre-selected, cryopreserved primary human CD4 T cells from normal donors were obtained from Bloodworks (Seattle WA). Human T cells were cultured in OpTmizer medium (Thermo Fisher) supplemented with Immune Cell Serum Replacement (Thermo Fisher), 2 mM L-glutamine (Gibco), 2 mM Glutamax (Gibco), 200 IU/ml IL-2 (R&D systems), 120 IU/ml IL-7 (R&D systems), and 20 IU/ml IL-15 (R&D systems).

Lentivirus was produced using standard protocols in a HEK293T suspension line. Viral supernatant was concentrated 10× using Lenti-X (Takara Bio) following the manufacturer's protocol.

For lentiviral transduction, T cells were stimulated with a 1:100 dilution of T cell TransAct (Miltenyi) for 30 hours. Virus was then added to T cells for 18-24 hours. Stimulation and viral infection were then terminated by addition of 7 volumes of fresh media without TransAct, and cells were cultured for 3-7 additional days before analysis.

Purified recombinant Ror1 linked to human Ig Fc was produced in-house and conjugated to Alexa 647 dye for detection and used as a flow cytometry reagent to detect the ROR1 CAR.

Kill switch-positive cells (le5) were incubated for 24-48 hours with inducer drug before staining and analysis for death. Conditions were normalized to contain about 50% transduction positive (+) cells by adding untransduced Jurkat or primary T cells in order to get accurate statistics on death.

Flow cytometry was performed on a Ze5 cytometer (Biorad). To determine expression of markers, between 1×10⁵-2×10⁵ total cells were transferred to a U-bottom 96 well culture dish (Corning). Cells were washed twice with Cell Staining Buffer (BioLegend), then stained with Zombie Violet (1:1000, BioLegend) and ROR1-FC for ROR1-specific CAR constructs (20 min at room temperature). Cells were washed twice in Cell Staining Buffer, then stained with Annexin V-PE (BD) in 1× Annexin V Binding Buffer (BD) for 15 min at room temperature. After staining, cells were washed once with Annexin V Binding Buffer, resuspended in AnneinV Binding Buffer and immediately analyzed.

Flow cytometry data was analyzed using FlowJo 10 (Tree Star). Percent killing was calculated by gating on live cells, then calculating 100*((percent GFP positive DMSO condition)−(percent GFP positive drug condition))/(percent GFP positive DMSO condition).

For flow analysis of induction of CD69 expression, Jurkat cells were mixed 1:1 with Jeko1 cells or Jeko1-ROR1-KO cells. Drug was added to a final concentration of 500 nM. Cultures were incubated with drug for 24 hours before flow analysis for surface markers.

For IncuCyte analysis of Jeko1 killing by primary T cells, 5e4 Jeko1-NucLight Red target cells were mixed with 5e4 CAR-positive T cells in 96-well flat-bottomed plates. Drug was added to appropriate final concentration at time zero, and plates were imaged every 6 hours for NucLight Red signal from the Jeko1 cells. Supernatants for MSD analysis of cytokine release were taken at 24 hours post coculture setup. IL-2 and IFNγ were measured with a V-Plex Custom Human Cytokine kit (Meso Scale Discovery, K151AOH-4), following the manufacturer's protocol.

For IncuCyte analysis of CD3ζ-signaling tdTomato reporter line with CARs and DNCR2-casp-9 kill switch (with EGFP transduction maker), 5e4 CAR+ cells were plated per well in poly-D-Lys coated plate in which 5e4 Jeko1 cells were previously seeded. Drug (e.g., grazoprevir, danoprevir) was added to given final concentration and cells were imaged for tdTomato and EGFP signal at 20×, 800 msec exposure, 4 images/well, every 3 hours. Cells were removed from the Incucyte plate to a v-bottom plate for 2 washes in PBS at 48 hours when the drug condition was changed.

Coculture for flow analysis of the CAR/kill switch constructs was setup 24 hours before analysis. CAR-positive cells (le5 cells; part I and part II positive, i.e., ROR1+ and EGFRt+ (part II marker)) were mixed with le5 Jeko1 cells and drug. Cells were analyzed for ROR1, EGFRt (MLLLVTSLLLCELPHPAFLLIPRKVCNGIGIGE), tdTomato, EGFP, and viability as described hereinbelow.

Percent killing was calculated by gating on live cells, then calculating 100*((percent GFP positive DMSO condition)−(percent GFP positive drug condition))/(percent GFP positive DMSO condition).

Flow cytometry of CAR experiments was performed on a Ze5 cytometer (Biorad). Purified recombinant Ror linked to human Ig Fc was produced in-house and conjugated to Alexa 647 dye for detection and used as a flow cytometry regent to detect the ROR1 CAR. To determine expression of markers, between 1×10⁵-2×10⁵ total cells were transferred to a U-bottom 96 well culture dish (Corning). Cells were washed twice with Cell Staining Buffer (BioLegend), then stained with the relevant reagents in a total volume of 25 μL Brilliant Stain Buffer (BD) for 30 minutes on ice (for CD69 experiment). Alternatively, cells were stained in Cell Staining Buffer, with eFluor 780 fixable viability dye (eBioscience) at 1:1000 dilution. After staining, cells were washed twice with Cell Staining Buffer, optionally fixed in FluoroFix Buffer (Biolegend) and kept at 4° C. in the dark until analysis. Flow cytometry data was analyzed using FlowJo 10 (Tree Star).

Drugs used in the following examples and their target proteins are shown in Table 2. T cell markers and fluorochromes used for detection are shown in Table 3.

TABLE 2
Drugs used and their target proteins

| Drug | Source | Catalog number | Target proteins |
|---|---|---|---|
| danoprevir | ApexBio | A4024 | DNCR2 |
| grazoprevir | ApexBio | A3618 | ANR, GNCR1 |
| A-1155463 | ApexBio | B6163 | Bcl-xL |
| AP21967 | Takara | 635055 | FKBP/FRB(T2098L) |
| AP1903 | ApexBio | B4168 | FKBP-F36V |
| (rimiducid) | | | |
| rapamycin | ApexBio | A8167 | FKBP/FRB |

TABLE 3
T cell markers and fluorochromes used for detection

| Target | Fluorochrome | Clone | Supplier |
|---|---|---|---|
| CD69 | PE | FN50 | Biolegend |
| CD3 | BUV805 | SK7 | BD |
| EGFR | BV421 | AY13 | Biolegend |

### Temporal Control of CAR Activity

The three NS3a-based regulated CARs described hereinabove with reference to FIG. 1B were evaluated in the Jurkat cell line. The CARs are specific for the ROR1 antigen expressed on the target cell line Jeko1. In this example, the two polypeptide chains of the regulatable CARs were expressed from a single lentivirus separated by a P2a. We compared the performance of the three NS3a-based CARs to a chemically inducible (“on-switch”) dimer FKBP/AP21967/FRB(T2098L) system and a chemically disruptable (“off-switch”) dimer Bcl-xL/A-1155463/Bad. Briefly, Jurkat cells expressing the designed small molecule-regulatable CARs were mixed 1:1 with Jeko1 target cells in the absence or presence of 500 nM cognate small molecule drug. The same Jurkat cells were mixed 1:1 with Jeko1 knockout (KO) cells lacking ROR1 as a negative control. Untransduced Jurkat cells, cells expressing an on-switch CAR without the CD3ζ signaling domain, and a conventional single-chain ROR1-CAR were used as controls. CD69 expression, a T cell activation marker, was detected by flow cytometry, gating on live CD3+/ROR1+ cells.

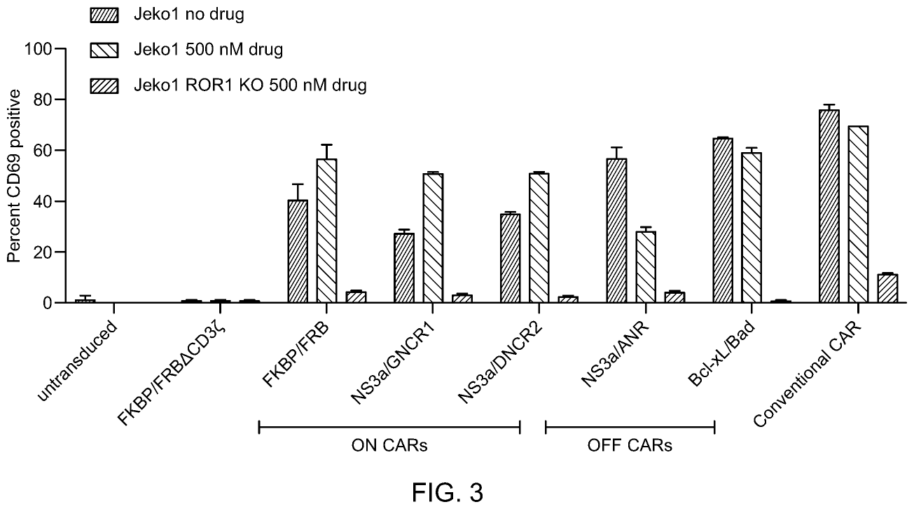

FIG. 3 is a plot showing small molecule drug-regulated induction of CD69 expression in CAR expressing Jurkat cells cocultured with Jeko1 cells or Jeko1-ROR1-KO cells. Jurkat cells expressing the regulatable CARs were mixed 1:1 with Jeko1 target cells in the absence (black bars) or presence (light gray bars) of 500 nM cognate small molecule drug. The same Jurkat cells were co-cultured with Jeko1 knockout (KO) cells lacking ROR1 as a negative control (dark gray bars). The data show that when Jurkat cells expressing the regulatable CARs are co-cultured 1:1 with Jeko1 target cells, CD69 expression was activated on Jurkat cells in response to CAR signaling, as indicated by flow cytometric analysis of Jurkat surface marker staining. All three on-switch CARs (“ON CARs” FKBP/FRB, NS3a/GNCR1, and NS3a/DNCR2) showed more cells with CD69 expression in the presence of their cognate inducer small molecule drug than in the absence of the small molecule drug. The ANR off-switch CAR (disrupted by grazoprevir) gave better performance than the Bcl-xL/Bad off-switch CAR, as the later showed little reduction in CD69 expression in the presence of disruptor drug. Untransduced Jurkats or Jurkats transduced with an on-switch CAR without the CD3ζ signaling domain (FKBP/FRBΔCD3ζ) showed no activation of CD69 expression. A conventional, single-chain ROR1-CAR (“Conventional CAR”) showed no significant regulation in response to 500 nM danoprevir. Comparison of the off-state of the regulatable CARs (no drug for on-switch CARs, with drug for off-switch CARs) to CD69 activation in response to target Jeko1 cells that have the ROR1 antigen knocked out (ROR1 KO) shows that none of these CARs achieve a complete off-state.

We next evaluated the regulatable CARs in a 1:1 mix of CD4+ and CD8+ primary human T cells. In this example, we expressed the two polypeptide chains from two separate lentiviral vectors to reduce any background that might arise from incomplete skipping of the P2a element in a single-vector construct described with reference to FIG. 3. Briefly, T cells expressing both parts of the regulatable CARs were mixed 1:1 with Jeko1 target cells that contained a NucLight Red marker for tracking of target cell growth in the presence or absence of 500 nM an inducer or disruptor drug. The drugs used were A-1155463 for the Bcl-xL/Bad dimer, grazoprevir for the NS3a/ANR dimer, grazoprevir for the NS3a/GNCR1 dimer, and danoprevir for the NS3a/DNCR2 dimer. Untransduced cells and cells expressing a conventional single-chain ROR1-CAR were used as controls. Cell growth was tracked using an IncuCyte killing assay. Cytokine production from the CAR T cells were measured by taking supernatants at 24 hours and using a custom V-plex kit (Meso Scale Discovery, MSD).

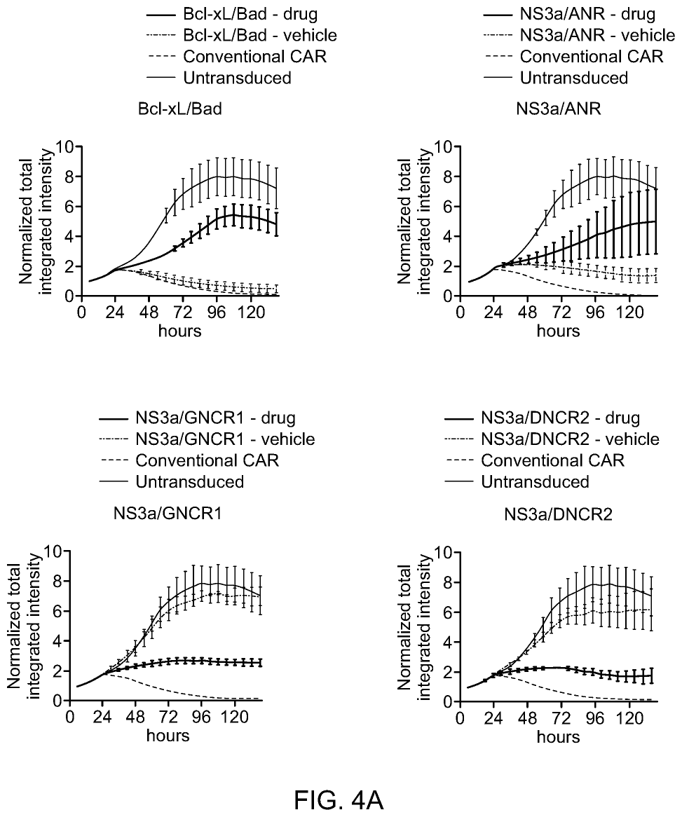

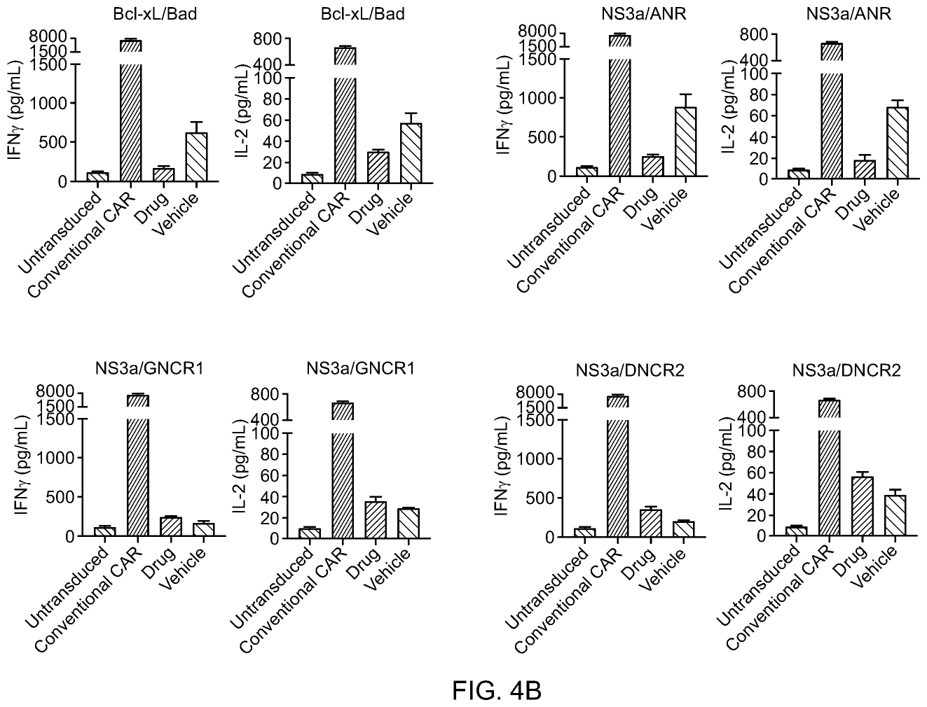

FIG. 4A is a panel of plots 400, 410, 415, and 420 showing target cell killing by the regulatable CARs in the presence or absence of their cognate inducer or disruptor small molecule drug. The data show that the on-switch CARs, GNCR1-CAR (plot 415) and DNCR2-CAR (plot 420), have very little activity in the off-state (“vehicle” control lacking the drug), with comparable lack of target cell killing to untransduced T cells. However, in the presence of their respective inducer small molecule drug the on-switch CARs show less control of Jeko1 growth than the conventional ROR1 CAR. The off-switch CARs, Bcl-xL/BAD (plot 400) and NS3a/ANR (plot 410) show control of target cells growth close to that of the conventional CAR, but a leakier off-state with more killing of target cells than from untransduced T cells.

FIG. 4B is a panel of plots 425, 430, 435, and 440 showing production of the cytokines IFNγ and IL-2 from the T cells expressing Bcl-xL/Bad, NS3a/ANR, NS3a/GNCR1, or NS3a/DNCR2, respectively, of FIG. 4A. The data show that most of the trends in cytokine production for each regulatable CAR were in the correct direction of modulation, but the regulatable CARs resulted in lower overall cytokine production than the conventional CAR.

Because our NS3a-based dimerization systems can respond to two different inducer drugs, danoprevir for DNCR2 and grazoprevir for GNCR1, we can use the two drug inputs to control two different signaling outputs. In one example, a dual-function CAR system includes an activation or on-switch component to activate CAR signaling and a safety-switch component to induce apoptosis.

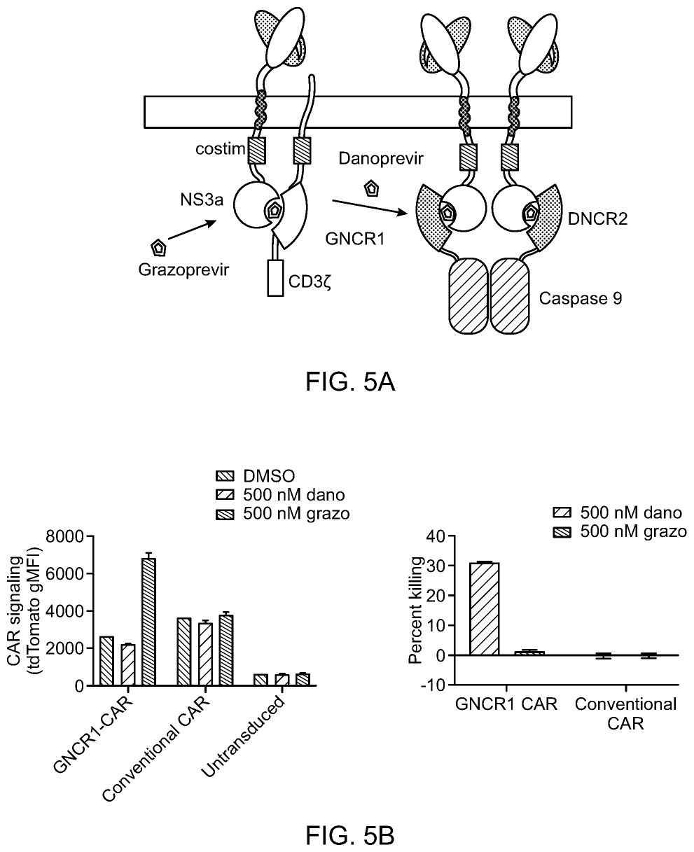

FIG. 5A is a schematic diagram of a dual-function on-switch/safety-switch regulatable T cell CAR. The figure illustrates expressed on-switch components 510 (NS3a/CAR and GNCR1-CD3ζ) of the system interacting in the presence of the small molecule inducer grazoprevir. Activation of the CAR induces receptor dimerization. When the small molecule danoprevir is added, it out-competes grazoprevir, recruiting the safety-switch component 515 (DNCR2-caspase-9) to the homodimeric CAR fused to NS3a. The homodimerized NS3a results in dimerization and activation of caspase-9 yielding T cell death.

To evaluate the use of two drug inputs to control two different signaling outputs, we used a Jurkat cell CD3ζ-signaling reporter line expressing the red fluorescent protein tdTomato to track CAR signaling, and the loss of the transduction marker (EGFP) of the DNCR2-caspase-9 construct to track Jurkat cell death.

FIG. 5B is a pair of plots 520 and 525 showing CAR signaling and percent killing, respectively, in the Jurkat CD3ζ-signaling reporter line in the presence and absence of grazoprevir or danoprevir. Referring now to plot 520, the data show that the dual-function CAR is activated by 500 nM grazoprevir (“grazo”), but not by 500 nM danoprevir (“dano”) as measured by tdTomato gMFI. Referring now to plot 525, the data show that the safety-switch in the GNCR1-CAR/DNCR2-casp-9 system is triggered by 500 nm danoprevir (dano) as indicated by the increase in percent killing, but not by 500 nM grazoprevir (grano). In this example, percent killing of the Jurkat cells was measured by loss of the transduction marker EGFP-positive population in the drug-treated population relative to a DMSO-treated control. Grazoprevir and danoprevir had no effect on Jurkat cells expressing a conventional CAR with DNCR2-casp-9.

We tested the ability to switch between CAR signaling and induction of apoptosis over time in an IncuCyte assay. Briefly, the CD3ζ-signaling reporter line expressing the dual-function GNCR1-CAR/DNCR2-casp9 was mixed 1:1 with Jeko1 cells and treated with 500 nM grazoprevir or danoprevir, or DMSO. Over a period of 48 hours, CAR signaling was tracked by tdTomato intensity and drug-induced cell death (i.e., activation of the safety switch) was monitored by tracking the transduction marker EGFP intensity.

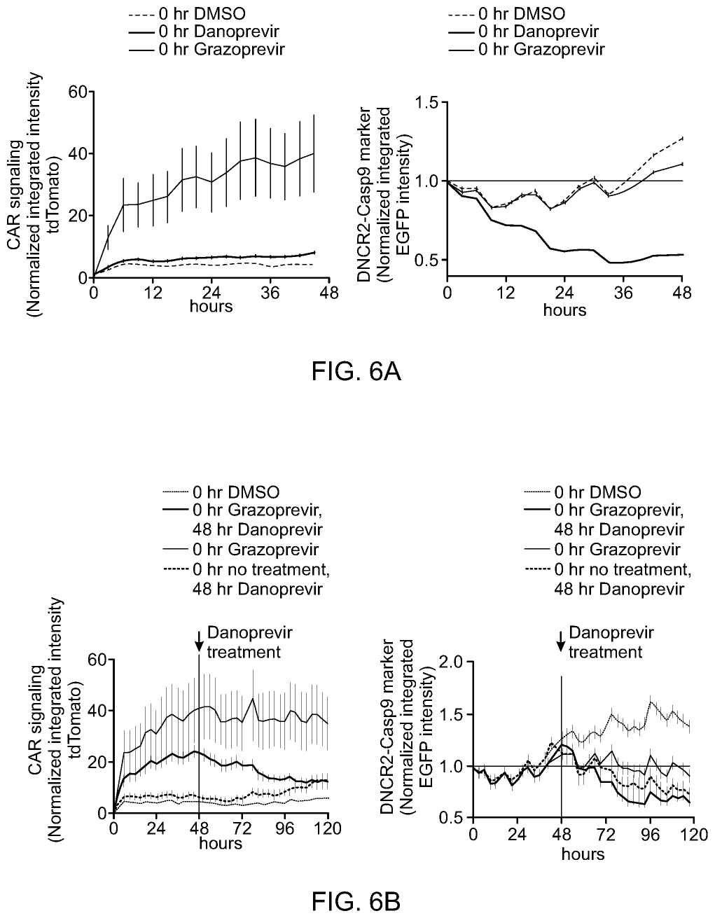

FIG. 6A is a pair of plots 600 and 610 showing CAR signaling and drug-induced cell death, respectively, in the Jurkat CD3ζ-signaling reporter line in the presence and absence of grazoprevir or danoprevir. The data show that over 48 hours CAR signaling (plot 600) was only activated by grazoprevir, as designed, and that only danoprevir treatment resulted in decreased cell viability as indicated by a loss of EGFP intensity (plot 610).

We then tested the ability to switch from induction of CAR signaling with grazoprevir to induction of apoptosis with danoprevir. Briefly, CAR signaling was activated by grazoprevir treatment at time zero. After 48 hours, the cells were washed and treated with danoprevir. CAR signaling (tdTomato intensity) and cell viability (EGFP intensity) were tracked over an additional 72-hour period.

FIG. 6B is a pair of plots 615 and 620 showing CAR signaling and drug-induced cell death, respectively, in Jurkat CD3ζ-signaling reporter cells switched from grazoprevir treatment to danoprevir treatment. Grazoprevir treatment was added at time zero (solid line), with no grazoprevir treatment (black dashed line) as controls. At 48 hours (indicated by arrow), cells were washed and treated with danoprevir (black solid line) or no treatment (gray line). The data show that CAR signaling (plot 615) increases after grazoprevir treatment and declines after danoprevir treatment. The data also show that cell viability remained constant until treatment with danoprvir at 48 hours, at which point the EGFP signal began to drop both for cells that had previously been treated with grazoprevir (black solid line) and for cells that were not treated with grazoprevir (black dashed line).

Referring now to FIG. 6A and FIG. 6B, the data demonstrate the ability to temporally switch between the control of two different signaling outputs. The small molecule regulation of CAR signaling could serve as a safety switch to pause CAR-T cell activity, while the inducible caspase-9 switch would act as a terminal safety switch to destroy the T cell therapy.

### 1.1.4. Small Molecule Drug-Induced Cell Death

The utility of cell and gene therapies can be enhanced by safety mechanisms that enable specific induction of cell death in the therapeutic cells in the event of adverse reactions in patients. We designed several different strategies to activate cell death through small molecule drug-induced homo-oligomerization of killing domains as described herein above with reference to FIG. 2. These strategies included direct dimerization via the inducible heterodimer or homo-oligomerizing one half of the inducible heterodimer via fusion to a homo-oligomeric scaffold (e.g., CAR) or a homo-oligomeric helical bundle.

We first tested for drug-induced death of Jurkat cells by fusing NS3a and DNCR2 separately to the catalytic domain of caspase-9 (NCDC). The small molecule inducible homodimer FKBP-F36V (iCasp9), which is dimerized by AP1903 (rimiducid) and the inducible heterodimer FKBP/rapamycin/FRB dimer (rapaCasp9) were used as controls. Briefly, Jurkat cells expressing NCDC, iCasp9, or rapaCasp9 were exposed to danoprevir, rimiducid or rapamycin, respectively, for 24 hours and analyzed by flow cytometry with gating set to measure killing in the top about 10% of cells expressing the highest amount of the GFP kill switch transduction marker.

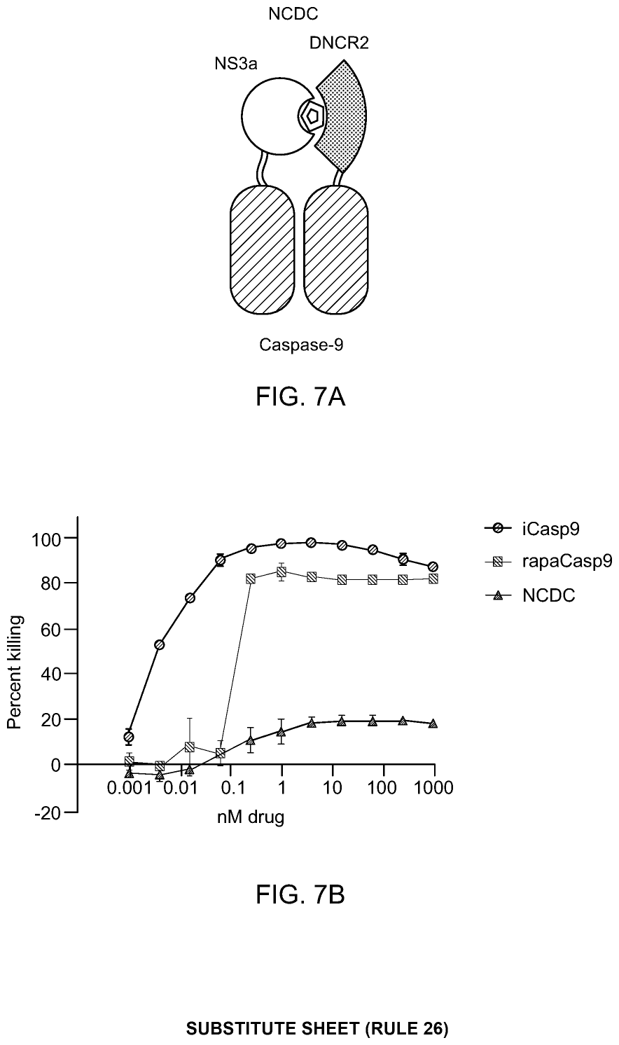

FIG. 7A is a schematic diagram of a two-chain direct dimerization design NCDC with caspase-9 killing domains. FIG. 7B is a plot showing percent killing as a function of drug concentration for the two-chain direct dimerization design NCDC. The data show that titration of the dimerizing drug danoprevir resulted in modest killing of the most highly-expressing Jurkat cells. In comparison, iCasp9 or rapaCasp9 killed about 80-100% of this high-expressing population in response to rimiducid or rapamycin, respectively.

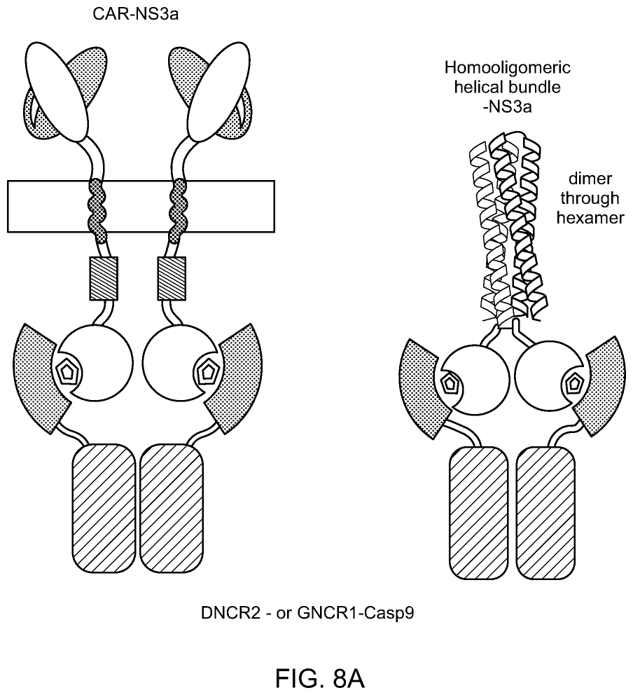

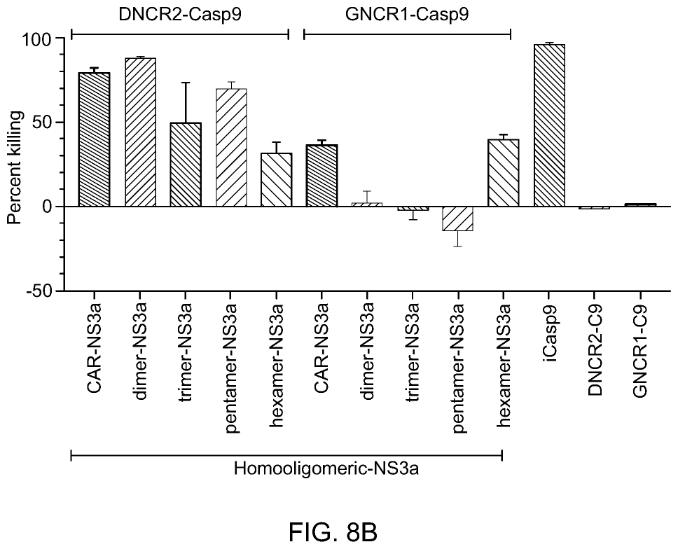

To reduce the large genetic cargo that comes from encoding the caspase-9 domain twice in the NS3a-casp-9/DNCR2-casp-9 kill switch construct, we tried strategies that involved templated homo-oligomerization of NS3a. FIG. 8A is a schematic diagram of CAR-NS3a-casp9 and homo-oligomeric helical bundle-NS3a caspase-9 kill switches. NS3a was fused to the C-terminus of a CAR construct (CAR-NS3a) or to the C-terminus of designed, homo-oligomeric helical bundles, including a dimer, trimer, pentamer, and hexamer. For both kill switches, caspase-9 was fused to DNCR2 or GNCR1.

To evaluate the kill switch designs of FIG. 8A, constructs were expressed in Jurkat cells, treated with 500 nM danoprevir (DNCR2-casp-9) or 500 nM grazoprevir (GNCR1-casp-9) and cell death measured by percent killing as described herein above.

FIG. 8B is a plot showing percent killing activity of the templated dimerization designs of FIG. 8A. The data show that when expressed in Jurkats, treatment with 500 nM danoprevir (DNCR2-casp-9) or 500 nM grazoprevir (GNCR1-casp-9) induced Jurkat death to varying degrees. All homo-oligomeric scaffolds of NS3a induced robust death with DNCR2-casp-9, but only the CAR-NS3a and hexamer-NS3a induced modest death with GNCR1-casp-9. This may be due to the lower affinity of GNCR1 for the grazoprevir/NS3a complex when compared to the high affinity of DNCR2 for the danoprevir/NS3a complex (see Foight, G. W. et al., Nature Biotechnology (2019) 37: 1209-1216). The best two scaffolds, CAR-NS3a and dimer-NS3a with DNCR2-casp-9 induced comparable killing to iCasp9. DNCR2-casp-9 and GNCR1-casp-9 with danoprevir or grazoprevir treatment did not induce Jurkat death in the absence of a scaffolded NS3a.

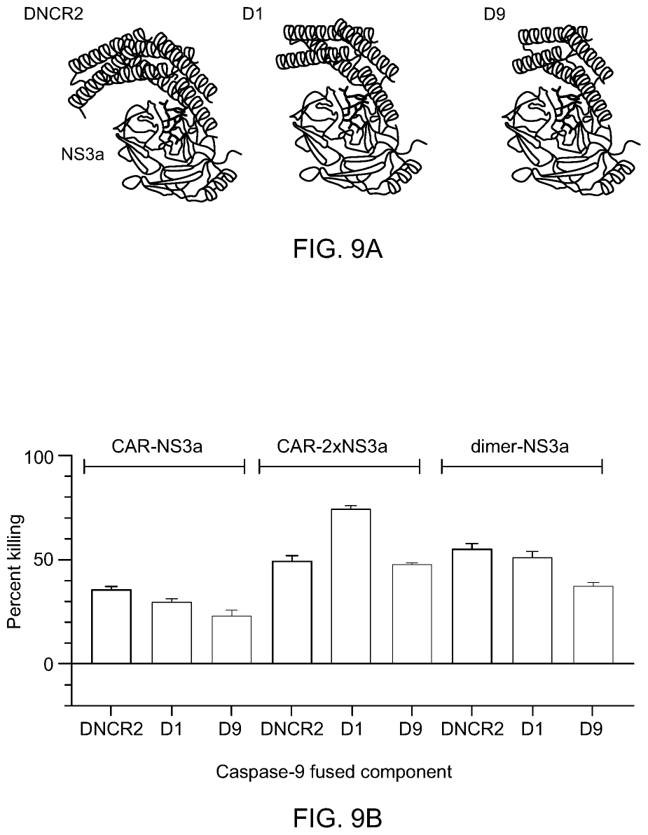

To reduce the size of CAR constructs further, we used Rosetta protein design to design minimized versions of DNCR2 by trimming the length and number of helices. FIG. 9A is a schematic diagram of models for minimized DNCR2 versions D1 and D9. Models for D1 and D2 are shown in complex with danoprevir/NS3a next to the full length DNCR2/danoprevir/NS3a.

The miniaturized versions of DNCR2, D1 and D9, were fused to caspase-9 and co-expressed in Jurkat cells with homo-oligomerized NS3a scaffolds. We also evaluated a CAR construct that included an additional copy of NS3a (CAR-2×NS3a). The original DNCR2-casp-9 was used as a control. Jurkat cells expressing different DNCR2 variants fused to caspase-9 along with CAR-NS3a, CAR-NS3a-NS3a, or dimer-NS3a were treated with 500 nM danoprevir and cell death was measured by percent killing as described herein above.

FIG. 9B is a plot showing percent killing activity of the miniaturized designs of FIG. 9A. The data show that when fused to caspase-9 and co-expressed with homo-oligomerized NS3a scaffolds, the minimized DNCR2 versions D1 and D9 induced comparable Jurkat death as the original DNCR2-casp-9. We increased killing further by fusing an additional copy of NS3a to the CAR construct (CAR-2×NS3a).

We explored the ability of other human killing domains to induce cell death in Jurkat and primary human T cells. We fused FKBP-F36V to a panel of killing domains that included caspase-9 D330A, caspase-9, caspase-1, caspase-4, and the necroptosis-inducing protein RIPK3. Briefly, Jurkat and primary human CD4+/CD8+ T cells expressing FKBP(F36V)-fused killing domains were treated with 10 nM rimiducid and cell death was measured by percent killing as described herein above.

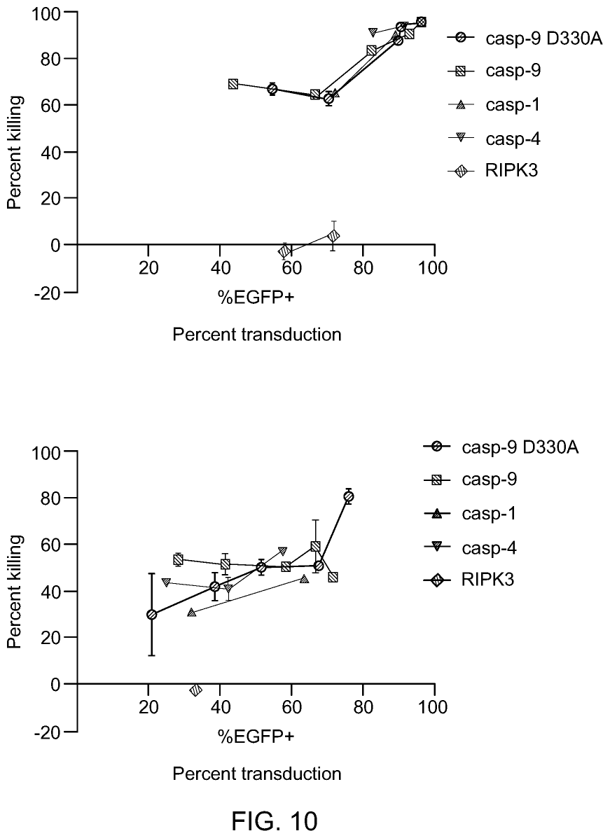

FIG. 10 is a pair of plots and showing percent killing in Jurkat and primary T cells, respectively, expressing the FKBP(F36V)-fused killing domains at a range of transduction levels. Percent EGFP (% EGFP) denotes different transduction levels of the killing domains. The data show that similar killing was induced by caspase-1, caspase-4, caspase-9 and a low basal-activity mutant of caspase-9, D330A in both cell types. This killing was proportional to the transduction level of the cells with the lentiviral construct expressing the kill switch, as measured by the percent of cells that were positive for the EGFP transduction marker. RIPK3, a necroptosis-inducing protein was not able to induce cell death in this assay. By extension, caspase-1 and caspase-4 would likely function similarly to caspase-9 to induce cell death with our DNCR/NS3a dimeric systems.

1. A fusion protein comprising a chimeric polypeptide operably-linked to a dimerization domain, wherein the chimeric polypeptide comprises an extracellular domain comprising an antigen sensing domain, a transmembrane domain, and an intracellular domain, and wherein the the chimeric polypeptide is operably linked to the dimerization domain intracellularly.

2.-47. (canceled)

48. A nucleic acid encoding the fusion protein of claim 1.

49.-55. (canceled)

56. A vector comprising the nucleic acid of claim 48.

57.-61. (canceled)

62. A cell comprising a fusion protein of claim 1.

63.-72. (canceled)

73. A composition comprising a fusion protein of claim 1.

74. A pharmaceutical composition comprising a composition of claim 73 and a pharmaceutically-acceptable carrier.

75. A second fusion protein comprising a signaling domain operably-linked to a dimerization domain, wherein,
(a) in the presence of a small molecule inducer, the dimerization domain is capable of forming a dimer with the dimerization domain of a first fusion protein of claim 1 or
(b) in the presence of an inhibitor, a dimer comprising the dimerization domain of the second fusion protein and the dimerization domain of the first fusion protein may be disrupted.

76.-103. (canceled)

104. A nucleic acid encoding the second fusion protein of claim 75.

105.-111. (canceled)

112. A vector comprising the nucleic acid of claim 104.

113-117. (canceled)

118. A cell comprising a fusion protein of claim 75.

119.-137. (canceled)

138. A composition comprising a fusion protein of claim 75.

139.-147. (canceled)

148. A pharmaceutical composition comprising a composition of claim 138 and a pharmaceutically-acceptable carrier.

149.-154. (canceled)

155. A method of treating a disease or a disorder, comprising administering to a subject an effective amount of a fusion protein of claim 1, wherein a severity of a sign or symptom of the disease or disorder is decreased, thereby treating the disease or disorder.

156. (canceled)

157. The method of claim 155, wherein the disease or disorder comprises one or more of an autoimmune disease or disorder; an inflammatory’ disease or disorder; an immunodeficiency disease or disorder; an ischemic disease or disorder; a blood disease or disorder; a bone disease or disorder; a neurological disease or disorder; a cardiac disease or disorder, a vascular disease or disorder; a metabolic disease or disorder; a dermatological disease or disorder; a digestive disease or disorder; a mitochondrial disease or disorder; a muscle disease or disorder; a liver disease or disorder; a kidney disease or disorder; a hearing disease or disorder; an ophthalmic disease or disorder; and a proliferative disease or disorder.

158.-160. (canceled)

161. A cell comprising the nucleic acid of claim 48.

162. A cell comprising the vector of claim 56.

163. A composition comprising the nucleic acid of claim 48.

164. A composition comprising the vector of claim 56.

165. A composition comprising the cell of claim 62.

166. A cell comprising the nucleic acid of claim 104.

167. A cell comprising the vector of claim 112.

168. A composition comprising the nucleic acid of claim 104.

169. A composition comprising the vector of claim 112.

170. A composition comprising the cell of claim 118.

171. A pharmaceutical composition comprising the composition of claim 168.

172. A pharmaceutical composition comprising the composition of claim 169.

173. A pharmaceutical composition comprising the composition of claim 170.

174. A method of treating a disease or a disorder, comprising administering to a subject an effective amount of the nucleic acid of claim 48, wherein a severity of a sign or symptom of the disease or disorder is decreased, thereby treating the disease or disorder.

175. A method of treating a disease or a disorder, comprising administering to a subject an effective amount of the vector of claim 56, wherein a severity of a sign or symptom of the disease or disorder is decreased, thereby treating the disease or disorder.

176. A method of treating a disease or a disorder, comprising administering to a subject an effective amount of the cell of claim 62, wherein a severity of a sign or symptom of the disease or disorder is decreased, thereby treating the disease or disorder.

177. A method of treating a disease or a disorder, comprising administering to a subject an effective amount of the composition of claim 73, wherein a severity of a sign or symptom of the disease or disorder is decreased, thereby treating the disease or disorder.

178. A method of treating a disease or a disorder, comprising administering to a subject an effective amount of the pharmaceutical composition of claim 74, wherein a severity of a sign or symptom of the disease or disorder is decreased, thereby treating the disease or disorder.


SEQUENCE LISTING (147 sequences)

SEQ ID NO: 1 - protein, 708 aa, Artificial Sequence
  Synthetic polypeptide
  NON_CONS (20)..(21): Residues at these positions can be separated by a scFV_hinge region of unknown length
MVLQTQVFISLLLWISGAYGCPPCPMFWVLVVVGGVLACYSLLVTVAFIIFWVKRGRKKLLYIFKQPFMRPVQTTQEEDGCSCRFPEEEEGGCELASGSASGSASMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGSGATNFSLLKQAGDVEENPGPMIHLGHILFLLLLPVAAAQTTPGERSSLPAFYPGTSGSCSGCGSLSLPMFWVLVVVGGVLACYSLLVTVAFIIFWVKRGRKKLLYIFKQPFMRPVQTTQEEDGCSCRFPEEEEGGCELGSASGSASGSASGSGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGSASGSASAGVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 2 - protein, 820 aa, Artificial Sequence
  Synthetic polypeptide
  NON_CONS (20)..(21): Residues at these positions can be separated by a scFV_hinge region of unknown length
MVLQTQVFISLLLWISGAYGCPPCPMFWVLVVVGGVLACYSLLVTVAFIIFWVKRGRKKLLYIFKQPFMRPVQTTQEEDGCSCRFPEEEEGGCELASGSASGSASMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGSGATNFSLLKQAGDVEENPGPMIHLGHILFLLLLPVAAAQTTPGERSSLPAFYPGTSGSCSGCGSLSLPMFWVLVVVGGVLACYSLLVTVAFIIFWVKRGRKKLLYIFKQPFMRPVQTTQEEDGCSCRFPEEEEGGCELGSASGSASGSASGSGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGSASGSASAGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 3 - protein, 1049 aa, Artificial Sequence
  Synthetic polypeptide
  NON_CONS (20)..(21): Residues at these positions can be separated by a scFV_hinge region of unknown length
MVLQTQVFISLLLWISGAYGCPPCPMFWVLVVVGGVLACYSLLVTVAFIIFWVKRGRKKLLYIFKQPFMRPVQTTQEEDGCSCRFPEEEEGGCELASGSASGSASMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSGATNFSLLKQAGDVEENPGPMIHLGHILFLLLLPVAAAQTTPGERSSLPAFYPGTSGSCSGCGSLSLPMFWVLVVVGGVLACYSLLVTVAFIIFWVKRGRKKLLYIFKQPFMRPVQTTQEEDGCSCRFPEEEEGGCELGSASGSASGSASGSGSDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSGSASGSASAGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 4 - protein, 1045 aa, Artificial Sequence
  Synthetic polypeptide
  NON_CONS (20)..(21): Residues at these positions can be separated by a scFV_hinge region of unknown length
MVLQTQVFISLLLWISGAYGCPPCPMFWVLVVVGGVLACYSLLVTVAFIIFWVKRGRKKLLYIFKQPFMRPVQTTQEEDGCSCRFPEEEEGGCELASGSASGSASMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSGATNFSLLKQAGDVEENPGPMIHLGHILFLLLLPVAAAQTTPGERSSLPAFYPGTSGSCSGCGSLSLPMFWVLVVVGGVLACYSLLVTVAFIIFWVKRGRKKLLYIFKQPFMRPVQTTQEEDGCSCRFPEEEEGGCELGSASGSASGSASGSGSSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHGSASGSASAGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 5 - protein, 849 aa, Artificial Sequence
  Synthetic polypeptide
  NON_CONS (20)..(21): Residues at these positions can be separated by a scFV_hinge region of unknown length
MVLQTQVFISLLLWISGAYGCPPCPMFWVLVVVGGVLACYSLLVTVAFIIFWVKRGRKKLLYIFKQPFMRPVQTTQEEDGCSCRFPEEEEGGCELASGSASGSASMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSGATNFSLLKQAGDVEENPGPMIHLGHILFLLLLPVAAAQTTPGERSSLPAFYPGTSGSCSGCGSLSLPMFWVLVVVGGVLACYSLLVTVAFIIFWVKRGRKKLLYIFKQPFMRPVQTTQEEDGCSCRFPEEEEGGCELGSASGSASGSASGSGSGELDELVYLLDGPGYDPIHSDVVTRGGSHLFNFGSASGSASAGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 6 - protein, 844 aa, Artificial Sequence
  Synthetic polypeptide
  NON_CONS (20)..(21): Residues at these positions can be separated by a scFV_hinge region of unknown length
MVLQTQVFISLLLWISGAYGCPPCPMFWVLVVVGGVLACYSLLVTVAFIIFWVKRGRKKLLYIFKQPFMRPVQTTQEEDGCSCRFPEEEEGGCELASGSASGSASMSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNGSGATNFSLLKQAGDVEENPGPMIHLGHILFLLLLPVAAAQTTPGERSSLPAFYPGTSGSCSGCGSLSLPMFWVLVVVGGVLACYSLLVTVAFIIFWVKRGRKKLLYIFKQPFMRPVQTTQEEDGCSCRFPEEEEGGCELGSASGSASGSASGSGSAPPNLWAAQRYGRELRRMSDEFEGSFKGSASGSASAGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 7 - protein, 207 aa, Artificial Sequence
  Synthetic polypeptide
  NON_CONS (20)..(21): Residues at these positions can be separated by a scFV_hinge region of unknown length
MVLQTQVFISLLLWISGAYGCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 662 aa, Artificial Sequence
  Synthetic polypeptide
MIHLGHILFLLLLPVAAAQTTPGERSSLPAFYPGTSGSCSGCGSLSLPMFWVLVVVGGVLACYSLLVTVAFIIFWVKRGRKKLLYIFKQPFMRPVQTTQEEDGCSCRFPEEEEGGCELGSASGSASGSASGSGSAPPNLWAAQRYGRELRRMSDEFEGSFKGSASGSASAGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 9 - protein, 668 aa, Artificial Sequence
  Synthetic polypeptide
MIHLGHILFLLLLPVAAAQTTPGERSSLPAFYPGTSGSCSGCGSLSLPMFWVLVVVGGVLACYSLLVTVAFIIFWVKRGRKKLLYIFKQPFMRPVQTTQEEDGCSCRFPEEEEGGCELGSASGSASGSASGSGSGELDELVYLLDGPGYDPIHSDVVTRGGSHLFNFGSASGSASAGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 10 - protein, 864 aa, Artificial Sequence
  Synthetic polypeptide
MIHLGHILFLLLLPVAAAQTTPGERSSLPAFYPGTSGSCSGCGSLSLPMFWVLVVVGGVLACYSLLVTVAFIIFWVKRGRKKLLYIFKQPFMRPVQTTQEEDGCSCRFPEEEEGGCELGSASGSASGSASGSGSSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHGSASGSASAGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 11 - protein, 868 aa, Artificial Sequence
  Synthetic polypeptide
MIHLGHILFLLLLPVAAAQTTPGERSSLPAFYPGTSGSCSGCGSLSLPMFWVLVVVGGVLACYSLLVTVAFIIFWVKRGRKKLLYIFKQPFMRPVQTTQEEDGCSCRFPEEEEGGCELGSASGSASGSASGSGSDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSGSASGSASAGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 12 - protein, 585 aa, Artificial Sequence
  Synthetic polypeptide
  NON_CONS (20)..(21): Residues at these positions can be separated by a scFV_hinge region of unknown length
MVLQTQVFISLLLWISGAYGCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 13 - protein, 303 aa, Artificial Sequence
  Synthetic polypeptide
  NON_CONS (20)..(21): Residues at these positions can be separated by a scFV_hinge region of unknown length
MVLQTQVFISLLLWISGAYGCPPCPMFWVLVVVGGVLACYSLLVTVAFIIFWVKRGRKKLLYIFKQPFMRPVQTTQEEDGCSCRFPEEEEGGCELASGSASGSASMSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNN

SEQ ID NO: 14 - protein, 302 aa, Artificial Sequence
  Synthetic polypeptide
  NON_CONS (20)..(21): Residues at these positions can be separated by a scFV_hinge region of unknown length
MVLQTQVFISLLLWISGAYGCPPCPMFWVLVVVGGVLACYSLLVTVAFIIFWVKRGRKKLLYIFKQPFMRPVQTTQEEDGCSCRFPEEEEGGCELASGSASGSASMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 15 - protein, 781 aa, Artificial Sequence
  Synthetic polypeptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHSGSASGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTSGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 16 - protein, 400 aa, Artificial Sequence
  Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 17 - protein, 399 aa, Artificial Sequence
  Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKT

SEQ ID NO: 18 - protein, 515 aa, Artificial Sequence
  Synthetic polypeptide
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLEYSGGGSLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 19 - protein, 1027 aa, Artificial Sequence
  Synthetic polypeptide
MKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGEGRGSLLTCGDVEENPGPSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHSGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 20 - protein, 1031 aa, Artificial Sequence
  Synthetic polypeptide
MKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGEGRGSLLTCGDVEENPGPDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSSGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 21 - protein, 727 aa, Artificial Sequence
  Synthetic polypeptide
MKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGSSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 22 - protein, 731 aa, Artificial Sequence
  Synthetic polypeptide
MKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGSDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 23 - protein, 730 aa, Artificial Sequence
  Synthetic polypeptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHSGGSGSGSKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 24 - protein, 727 aa, Artificial Sequence
  Synthetic polypeptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHSGGGSKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 25 - protein, 725 aa, Artificial Sequence
  Synthetic polypeptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHSGSKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 26 - protein, 722 aa, Artificial Sequence
  Synthetic polypeptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 27 - protein, 626 aa, Artificial Sequence
  Synthetic polypeptide
MDDNEAEKLIREAEEAAKDAEEAAERTGDPEVRELARELVRLAKEAAEEVQRNPSSSDVNEALHSIVYAIEAAIFALEAAERTGDPEVRELARELVRLAVEAAEEVQRNPSSRNVEHALMRIVLAIYLAEENLREKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGSGGSGSASGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 28 - protein, 590 aa, Artificial Sequence
  Synthetic polypeptide
MELEEVLKDLEEALERLRNEELRRLIEEIAELLRRRDDHSIIYMIEALIFVIEAVERTGDERIEKLLEELIKRIRNGDDDSAIMRIVLAVYLIEENLREKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGSGGSGSASGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 29 - protein, 549 aa, Artificial Sequence
  Synthetic polypeptide
MYAKEAAEFAREAAERTGDPEVRELARELERLARRGSSEHALMRIVLAIYLAEENLREKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGSGGSGSASGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 30 - protein, 734 aa, Artificial Sequence
  Synthetic polypeptide
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSSGGSGSGSKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 31 - protein, 731 aa, Artificial Sequence
  Synthetic polypeptide
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSSGGGSKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 32 - protein, 729 aa, Artificial Sequence
  Synthetic polypeptide
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSSGSKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 33 - protein, 726 aa, Artificial Sequence
  Synthetic polypeptide
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 34 - protein, 520 aa, Artificial Sequence
  Synthetic polypeptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHSGSASGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 35 - protein, 520 aa, Artificial Sequence
  Synthetic polypeptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHSGSASGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 36 - protein, 425 aa, Artificial Sequence
  Synthetic polypeptide
MDDNEAEKLIREAEEAAKDAEEAAERTGDPEVRELARELVRLAKEAAEEVQRNPSSSDVNEALHSIVYAIEAAIFALEAAERTGDPEVRELARELVRLAVEAAEEVQRNPSSRNVEHALMRIVLAIYLAEENLRESGSASGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 37 - protein, 389 aa, Artificial Sequence
  Synthetic polypeptide
MELEEVLKDLEEALERLRNEELRRLIEEIAELLRRRDDHSIIYMIEALIFVIEAVERTGDERIEKLLEELIKRIRNGDDDSAIMRIVLAVYLIEENLRESGSASGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 38 - protein, 348 aa, Artificial Sequence
  Synthetic polypeptide
MYAKEAAEFAREAAERTGDPEVRELARELERLARRGSSEHALMRIVLAIYLAEENLRESGSASGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 39 - protein, 523 aa, Artificial Sequence
  Synthetic polypeptide
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSGSASGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLAAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 40 - protein, 523 aa, Artificial Sequence
  Synthetic polypeptide
MDIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKSGSASGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 41 - protein, 302 aa, Artificial Sequence; identical to SEQ ID NO: 14
  Synthetic polypeptide
  NON_CONS (20)..(21): Residues at these positions can be separated by a scFV_hinge region of unknown length

SEQ ID NO: 42 - protein, 281 aa, Artificial Sequence
  Synthetic polypeptide
MTRTEIIRELERSLREQEELAKRLKELLRELERLQREGSSDEDVRELLREIKELVEEIEKLAREQKYLVEELKRQDGSASGSASMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 43 - protein, 277 aa, Artificial Sequence
  Synthetic polypeptide
MTEDEIRKLRKLLEEAEKKLKKLEDKTRRSEEISKTDDDPKAQSLQLIAESLMLIAESLLIIAISLLLSSRNGSASGSASMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 44 - protein, 276 aa, Artificial Sequence
  Synthetic polypeptide
MTRRKQEMKRLKKEMEKIREETEEVKKEIEESKKRPQSESAKNLILIMQLLINQIRLLALQIRMLALQLQEGSASGSASMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 45 - protein, 281 aa, Artificial Sequence
  Synthetic polypeptide
MSEYEIRKALEELKASTAELKRATASLRASTEELKKNPSEDALVENNRLIVEHNAIIVENNRIIAAVLELIVRAIKGSASGSASMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 46 - protein, 509 aa, Artificial Sequence
  Synthetic polypeptide
  NON_CONS (20)..(21): Residues at these positions can be separated by a scFV_hinge region of unknown length
MVLQTQVFISLLLWISGAYGCPPCPMFWVLVVVGGVLACYSLLVTVAFIIFWVKRGRKKLLYIFKQPFMRPVQTTQEEDGCSCRFPEEEEGGCELASGSASGSASMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSSGSSGSSGGKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 47 - protein, 403 aa, Artificial Sequence
  Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGSGNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 48 - protein, 403 aa, Artificial Sequence
  Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGSGEAGPPESGESTDALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGANRGELWVRDSPASLEVASSQSSENLEEDAVYKTHVEKDFIAFCSSTPHNVSWRDSTMGSIFITQLITCFQKYSWCCHLEEVFRKVQQSFETPRAKAQMPTIERLSMTRYFYLFPGN

SEQ ID NO: 49 - protein, 393 aa, Artificial Sequence
  Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGMENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 50 - protein, 393 aa, Artificial Sequence
  Synthetic polypeptide
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGMENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPGCFNFLTKELYFYH

SEQ ID NO: 51 - protein, 678 aa, Artificial Sequence
  Synthetic polypeptide
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGSGRLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 52 - protein, 697 aa, Artificial Sequence
  Synthetic polypeptide
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQYLPPKCMQEIPQEQIKEIKKEQLSGSPWILLRENEVSTLYKGEYHRAPVAIKVFKKLQAGSIAIVRQTFNKEIKTMKKFESPNILRIFGICIDETVTPPQFSIVMEYCELGTLRELLDREKDLTLGKRMVLVLGAARGLYRLHHSEAPELHGKIRSSNFLVTQGYQVKLAGFELRKTQTSMSLGTTREKTDRVKSTAYLSPQELEDVFYQYDVKSEIYSFGIVLWEIATGDIPFQGCNSEKIRKLVAVKRQQEPLGEDCPSELREIIDECRAHDPSVRPSVDEILKKLSTFSKGSMLEGVQVETISPEGGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETGSGMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 53 - protein, 334 aa, Artificial Sequence
  Synthetic polypeptide
MDAKARNCLLQHREALEKDIKTSYIMDHMISDGFLTISEEEKVRNEPTQQQRAAMLIKMILKKDNDSYVSFYNALLHEGYKDLAALLHDGIPVVSSSSGKDSVSGITSGSMLEGVQVETISPEGGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETGSGMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 54 - protein, 523 aa, Artificial Sequence
  Synthetic polypeptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHTGSGGGSGNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 55 - protein, 523 aa, Artificial Sequence
  Synthetic polypeptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHTGSGGGSGEAGPPESGESTDALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGANRGELWVRDSPASLEVASSQSSENLEEDAVYKTHVEKDFIAFCSSTPHNVSWRDSTMGSIFITQLITCFQKYSWCCHLEEVFRKVQQSFETPRAKAQMPTIERLSMTRYFYLFPGN

SEQ ID NO: 56 - protein, 515 aa, Artificial Sequence
  Synthetic polypeptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHTGSGGGSGMENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 57 - protein, 515 aa, Artificial Sequence
  Synthetic polypeptide
MSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNHTGSGGGSGMENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPGCFNFLTKELYFYH

SEQ ID NO: 58 - protein, 683 aa, Artificial Sequence
  Synthetic polypeptide
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNH

SEQ ID NO: 59 - protein, 702 aa, Artificial Sequence
  Synthetic polypeptide
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQYLPPKCMQEIPQEQIKEIKKEQLSGSPWILLRENEVSTLYKGEYHRAPVAIKVFKKLQAGSIAIVRQTFNKEIKTMKKFESPNILRIFGICIDETVTPPQFSIVMEYCELGTLRELLDREKDLTLGKRMVLVLGAARGLYRLHHSEAPELHGKIRSSNFLVTQGYQVKLAGFELRKTQTSMSLGTTREKTDRVKSTAYLSPQELEDVFYQYDVKSEIYSFGIVLWEIATGDIPFQGCNSEKIRKLVAVKRQQEPLGEDCPSELREIIDECRAHDPSVRPSVDEILKKLSTFSKGSSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNH

SEQ ID NO: 60 - protein, 339 aa, Artificial Sequence
  Synthetic polypeptide
MDAKARNCLLQHREALEKDIKTSYIMDHMISDGFLTISEEEKVRNEPTQQQRAAMLIKMILKKDNDSYVSFYNALLHEGYKDLAALLHDGIPVVSSSSGKDSVSGITSGSSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNH

SEQ ID NO: 61 - protein, 543 aa, Artificial Sequence
  Synthetic polypeptide
MDAKARNCLLQHREALEKDIKTSYIMDHMISDGFLTISEEEKVRNEPTQQQRAAMLIKMILKKDNDSYVSFYNALLHEGYKDLAALLHDGIPVVSSSSGKDSVSGITSGSMKKKGSVVIVGRINLSGDTAYAQQTRGEEGCQEMSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPTGSGGGSSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNH

SEQ ID NO: 62 - protein, 1355 aa, Artificial Sequence
  Synthetic polypeptide
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNMKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSGEGRGSLLTCGDVEENPGPMSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNSSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNH

SEQ ID NO: 63 - protein, 33 aa, Artificial Sequence
  Synthetic polypeptide
MLLLVTSLLLCELPHPAFLLIPRKVCNGIGIGE

SEQ ID NO: 64 - protein, 5 aa, Artificial Sequence
  Synthetic peptide
GGGGS

SEQ ID NO: 65 - protein, 75 aa, Artificial Sequence
  Synthetic polypeptide
SEYEIRKALEELKASTAELKRATASLRASTEELKKNPSEDALVENNRLIVEHNAIIVENNRIIAAVLELIVRAIK

SEQ ID NO: 66 - protein, 75 aa, Artificial Sequence
  Synthetic polypeptide
TRTEIIRELERSLREQEELAKRLKELLRELERLQREGSSDEDVRELLREIKELVEEIEKLAREQKYLVEELKRQD

SEQ ID NO: 67 - protein, 70 aa, Artificial Sequence
  Synthetic polypeptide
TRRKQEMKRLKKEMEKIREETEEVKKEIEESKKRPQSESAKNLILIMQLLINQIRLLALQIRMLALQLQE

SEQ ID NO: 68 - protein, 72 aa, Artificial Sequence
  Synthetic polypeptide
TEDEIRKLRKLLEEAEKKLKKLEDKTRRSEEISKTDDDPKAQSLQLIAESLMLIAESLLIIAISLLLSSRNG

SEQ ID NO: 69 - protein, 196 aa, Artificial Sequence
  Synthetic polypeptide
MAKGSVVIVGRINLSGDTAYSQQTRGLLGCIITSATGRDKNQVDGEVQVLSTATQSFLATCVNGVCWTVYHGAGSKTLAGPKGPITQMYTNVDQDLVGWPAPPGARSMTPCTCGSSDLYLVTRHADVIPVRRRGDSRGSLLSPRPVSYLKGSAGGPLLCPSGHVVGIFRAAVCTRGVAKAVDFIPVESMETTMRSP

SEQ ID NO: 70 - protein, 197 aa, Artificial Sequence
  Synthetic polypeptide
MKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 71 - protein, 196 aa, Artificial Sequence
  Synthetic polypeptide
KKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 72 - protein, 229 aa, Artificial Sequence
  Synthetic polypeptide
SSDEEEARELIERAKEAAERAQEAAERTGDPRVRELARELKRLAQEAAEEVKRDPSSSDVNEALKLIVEAIEAAVDALEAAERTGDPEVRELARELVRLAVEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNH

SEQ ID NO: 73 - protein, 233 aa, Artificial Sequence
  Synthetic polypeptide
DIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKLCIKAASEAAEAASKAAELAQRHPDSQAARDAIKLASQAAEAVERAIWLAAENPNADIAKKCIKAASEAAEEASKAAEEAQRHPDSQKARDEIKEASQKAEEVKERCKS

SEQ ID NO: 74 - protein, 33 aa, Artificial Sequence
  Synthetic polypeptide
GELDELVYLLDGPGYDPIHSDVVTRGGSHLFNF

SEQ ID NO: 75 - protein, 22 aa, Artificial Sequence
  Synthetic polypeptide
GSGATNFSLLKQAGDVEENPGP

SEQ ID NO: 76 - protein, 20 aa, Artificial Sequence
  Synthetic polypeptide
GSGEGRGSLLTCGDVEENPG

SEQ ID NO: 77 - protein, 93 aa, Artificial Sequence
  Synthetic polypeptide
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 78 - DNA, 586 bp, Artificial Sequence
  Synthetic polynucleotide
cctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccc

SEQ ID NO: 79 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 80 - protein, 101 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 81 - protein, 136 aa, Homo sapiens
MLRLLLALNLFPSIQVTGNKILVKQSPMLVAYDNAVNLSYNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 82 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 83 - protein, 55 aa, Homo sapiens
MLRLLLALNLFPSIQVTGNKILVKQSPMLVAYDNAVNLSYNEKSNGTIIHVKGEE

SEQ ID NO: 84 - protein, 80 aa, Homo sapiens
MLRLLLALNLFPSIQVTGNKILVKQSPMLVAYDNAVNLSYNEKSNGTIIHVKGKHLCPSPLFPGPSKPYAPPRDFAAYRS

SEQ ID NO: 85 - protein, 234 aa, Homo sapiens
MPCGLSALIMCPKGMVAVVVAVDDGDSQALAGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 86 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 87 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 88 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 89 - protein, 163 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 91 - protein, 168 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKM

SEQ ID NO: 92 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 93 - protein, 133 aa, Homo sapiens
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 94 - protein, 36 aa, Artificial Sequence
  Synthetic polypeptide
QRMKQLEDKVEELLSKNYHLENEVARLKKLVGDAAR

SEQ ID NO: 95 - protein, 134 aa, Artificial Sequence
  Synthetic polypeptide
DDNEAEKLIREAEEAAKDAEEAAERTGDPEVRELARELVRLAKEAAEEVQRNPSSSDVNEALHSIVYAIEAAIFALEAAERTGDPEVRELARELVRLAVEAAEEVQRNPSSRNVEHALMRIVLAIYLAEENLRE

SEQ ID NO: 96 - protein, 105 aa, Artificial Sequence
  Synthetic polypeptide
EERELARELERLAKEAAEEVQRNPSSSDVNEALHSIEYAREAAEFAREAAERTGDPEVRELARELVRLAVEAAEEVQRNPSSRNVEHALMRIVLAIYLAEENLRE

SEQ ID NO: 97 - protein, 78 aa, Artificial Sequence
  Synthetic polypeptide
DENEAEHSRRYAKEAAEFAREAAERTGDPEVRELARELERLAREAAEEVQRNPSSRNVEHALMRIVLAIYLAEENLRE

SEQ ID NO: 98 - protein, 78 aa, Artificial Sequence
  Synthetic polypeptide
DENELEHIRRYLKEILEFVREVVERTGDEHIRRLLEELERLVKKVIEELKRRPDLHHNEHAFMRLVLAVYLAEENLRE

SEQ ID NO: 99 - protein, 78 aa, Artificial Sequence
  Synthetic polypeptide
DENELEHIRRYLKEVLEFVREILERTGDEEVKKLIEELERLVKKIEEELKRDPRRHHNEHAVMRLVLAVYLLEENLRE

SEQ ID NO: 100 - protein, 105 aa, Artificial Sequence
  Synthetic polypeptide
DEEELHKRVEELWKELHELVRRHSHDSDLNELLHIIEYLREVLEFVREVLERTGDEHIKRLLHELVKRIVETIEELKRNSHHSNNEHAVMRIVLAVYLAEENLRE

SEQ ID NO: 101 - protein, 105 aa, Artificial Sequence
  Synthetic polypeptide
DEEELERRLEELWRRIKERVERHSHDSDLNELLHIIEYLREVVEFVREVVERTGDEHIKRLLLELIREIVEVIEKLERNLHDSNNEHAVMRIVLAVYLAEENLRE

SEQ ID NO: 102 - protein, 99 aa, Artificial Sequence
  Synthetic polypeptide
EVEEVLKDLEEAIERLHDHEINRLAKEVLKILRKRDDHSIIYAVEVMIFVIEVLERTGDEKVRELIRELAEIIRRKGGTDGAVMRLVLAIYLLEENLRE

SEQ ID NO: 103 - protein, 98 aa, Artificial Sequence
  Synthetic polypeptide
ELEEVLKDLEEALERLRNEELRRLIEEIAELLRRRDDHSIIYMIEALIFVIEAVERTGDERIEKLLEELIKRIRNGDDDSAIMRIVLAVYLIEENLRE

SEQ ID NO: 104 - protein, 102 aa, Artificial Sequence
  Synthetic polypeptide
EAEEAAKDAEEAAERLHDPEVRELARELVRLAKETTHEDGLHSIVYAIEAAIFALEAAERTGDPEVRELARELVRLARNGDHSGALMRIVLAIYLAEENLRE

SEQ ID NO: 105 - protein, 98 aa, Artificial Sequence
  Synthetic polypeptide
EAEEAAKDAEEAAERLHDPEVRELARELVRLAKERDDHSIVYAIEAAIFALEAAERTGDPEVRELARELVRLAREGDHSSALMRIVLAIYLAEENLRE

SEQ ID NO: 106 - protein, 134 aa, Artificial Sequence
  Synthetic polypeptide
DDNEAEKLIREAEEVLKDIEEILERLHDEEIIRLVEEVQKILKELLERVHKHPHDSDLNELLHIIFYMMEVLIFVLEVLERTGDEEIRKLVWHIVRIILEILEELRRNPKHSNNEHALMRLLLAIYLLEENLRE

SEQ ID NO: 107 - protein, 109 aa, Artificial Sequence
  Synthetic polypeptide
EAEEAAKDAEEAAERLHDPEVRELARELVRLAKEVDDHSIVYAIEAAIFALEAAERTGDPEVRELARELVRLAVEAAEEVQRNPSSRNVEHALMRIVLAIYLAEENLRE

SEQ ID NO: 108 - protein, 109 aa, Artificial Sequence
  Synthetic polypeptide
EFEEVLKDLEEVVERLHNEEIRRLLERLIELLRRVRDHSFIYLVEAVIFVLEVLERTGDHRVEELLEKLVRLVIEVLEELERHPHKENNEHAAMRFVLAVYLIEENLRE

SEQ ID NO: 109 - protein, 109 aa, Artificial Sequence
  Synthetic polypeptide
EWEEVLKDLEEALERLHDEEIRRLLERVVELLREDTDHSIVYLIEALIFVIEAVERTGDERIHRLLEELVRRIIKVIEELHRHPHKENNEHAAMRFVLAVYLIEENLRE

SEQ ID NO: 110 - protein, 79 aa, Artificial Sequence
  Synthetic polypeptide
EERELARELERLARERRDHSIEYAREAAEFAREAAERTGDPEVRELARELVRLARNGSDEHALMRIVLAIYLAEENLRE

SEQ ID NO: 111 - protein, 94 aa, Artificial Sequence
  Synthetic polypeptide
EERELARELERLAKEAAEEVQRNPSSSDVNEALHSIEYAREAAEFAREAAERTGDPEVRELARELVRLARNGSDEHALMRIVLAIYLAEENLRE

SEQ ID NO: 112 - protein, 79 aa, Artificial Sequence
  Synthetic polypeptide
DEEEIRKRIEEQWRRDTDHSWEYLREVAEFAREILERTGDERIKELVLRILELIENGTDDHAIMRLVLALYLIEENLRE

SEQ ID NO: 113 - protein, 79 aa, Artificial Sequence
  Synthetic polypeptide
DEEELREEVEHLWREDTDHSWEYLREALEFLREIVERTGDERLRELLKKALELVRHGTDGHAIMRLVLAAYLLEENLRE

SEQ ID NO: 114 - protein, 57 aa, Artificial Sequence
  Synthetic polypeptide
YAKEAAEFAREAAERTGDPEVRELARELERLARRGSSEHALMRIVLAIYLAEENLRE

SEQ ID NO: 115 - protein, 57 aa, Artificial Sequence
  Synthetic polypeptide
YAKEVAEFVREVWERTGDERVEELLKKLEELRRNGSDEHALMRLVLAVYLIEENLRE

SEQ ID NO: 116 - protein, 57 aa, Artificial Sequence
  Synthetic polypeptide
YWKEVAEFLREVLERTGDEEVKKLLEEFEELRRRGSDEHALMRLVLAVYLIEENLRE

SEQ ID NO: 117 - protein, 96 aa, Artificial Sequence
  Synthetic polypeptide
DEEELERKIEEQVRKLREEVERRSHDSDLNEALHSMEYLREIAEFAREIVERTGDEELRKLLKEILRLIHENPEDVGHAIMRLILALYLLEENLRE

SEQ ID NO: 118 - protein, 164 aa, Artificial Sequence
  Synthetic polypeptide
DDNEAEKLIREAEEAAKDAEEAAERTGDPEVRELARELVRLAKEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRD

SEQ ID NO: 119 - protein, 135 aa, Artificial Sequence
  Synthetic polypeptide
EERELARELERLAKEAAEEVQRNPSSSDVNEALHSIEYAREAAEFAREAAERTGDPEVRELARELVRLAVEAAEEVQRNPSSRNVEHALMRIVLAIYLAEENLREAEESGDPEKREKARERVREAVERAEEVQRD

SEQ ID NO: 120 - protein, 108 aa, Artificial Sequence
  Synthetic polypeptide
DENEAEHSRRYAKEAAEFAREAAERTGDPEVRELARELERLAREAAEEVQRNPSSRNVEHALMRIVLAIYLAEENLREAEESGDPEKREKARERVREAVERAEEVQRD

SEQ ID NO: 121 - protein, 108 aa, Artificial Sequence
  Synthetic polypeptide
DENELEHIRRYLKEILEFVREVVERTGDEHIRRLLEELERLVKKVIEELKRRPDLHHNEHAFMRLVLAVYLAEENLREAEESGDPEKREKARERVREAVERAEEVQRD

SEQ ID NO: 122 - protein, 108 aa, Artificial Sequence
  Synthetic polypeptide
DENELEHIRRYLKEVLEFVREILERTGDEEVKKLIEELERLVKKIEEELKRDPRRHHNEHAVMRLVLAVYLLEENLREAEESGDPEKREKARERVREAVERAEEVQRD

SEQ ID NO: 123 - protein, 135 aa, Artificial Sequence
  Synthetic polypeptide
DEEELHKRVEELWKELHELVRRHSHDSDLNELLHIIEYLREVLEFVREVLERTGDEHIKRLLHELVKRIVETIEELKRNSHHSNNEHAVMRIVLAVYLAEENLREAEESGDPEKREKARERVREAVERAEEVQRD

SEQ ID NO: 124 - protein, 135 aa, Artificial Sequence
  Synthetic polypeptide
DEEELERRLEELWRRIKERVERHSHDSDLNELLHIIEYLREVVEFVREVVERTGDEHIKRLLLELIREIVEVIEKLERNLHDSNNEHAVMRIVLAVYLAEENLREAEESGDPEKREKARERVREAVERAEEVQRD

SEQ ID NO: 125 - protein, 164 aa, Artificial Sequence
  Synthetic polypeptide
DDNEAEKLIREAEEVLKDIEEILERLHDEEIIRLVEEVQKILKELLERVHKHPHDSDLNELLHIIFYMMEVLIFVLEVLERTGDEEIRKLVWHIVRIILEILEELRRNPKHSNNEHALMRLLLAIYLLEENLREAEESGDPEKREKARERVREAVERAEEVQRD

SEQ ID NO: 126 - protein, 139 aa, Artificial Sequence
  Synthetic polypeptide
EAEEAAKDAEEAAERLHDPEVRELARELVRLAKEVDDHSIVYAIEAAIFALEAAERTGDPEVRELARELVRLAVEAAEEVQRNPSSRNVEHALMRIVLAIYLAEENLREAEESGDPEKREKARERVREAVERAEEVQRD

SEQ ID NO: 127 - protein, 139 aa, Artificial Sequence
  Synthetic polypeptide
EFEEVLKDLEEVVERLHNEEIRRLLERLIELLRRVRDHSFIYLVEAVIFVLEVLERTGDHRVEELLEKLVRLVIEVLEELERHPHKENNEHAAMRFVLAVYLIEENLREAEESGDPEKREKARERVREAVERAEEVQRD

SEQ ID NO: 128 - protein, 139 aa, Artificial Sequence
  Synthetic polypeptide
EWEEVLKDLEEALERLHDEEIRRLLERVVELLREDTDHSIVYLIEALIFVIEAVERTGDERIHRLLEELVRRIIKVIEELHRHPHKENNEHAAMRFVLAVYLIEENLREAEESGDPEKREKARERVREAVERAEEVQRD

SEQ ID NO: 129 - protein, 174 aa, Artificial Sequence
  Synthetic polypeptide
TSSDEEEARELIEEAKEAAEDAQEAAERTGDPEVRELARELKRLAQEAAEEVQRNPSSSDVNEALHSIVYAIEAAIFALEAAERTGDPEVRELARELVRLAVEAAEEVQRNPSSRNVEHALMRIVLAIYLAEENLREAEESGDPEKREKARERVREAVERAEEVQRDPSGWLNH

SEQ ID NO: 130 - protein, 177 aa, Artificial Sequence
  Synthetic polypeptide
DIEKLCKKAEEEAKEAQEKADELRQRHPDSQAAEDAEDLANLAVAAVLTACLLAQEHPNADIAKLCIKAASEAAEAASKAAELAQRHPDSQAARDAIKLASQAARAVILAIMLAAENPNADIAKKCIKAASEAAEEASKAAEEAQRHPDSQKARDEIKEASQKAEEVERRCWRAAEN

SEQ ID NO: 131 - protein, 110 aa, Artificial Sequence
  Synthetic polypeptide
DIEKLCKKAEEEAKEEGNELAVAAVLTACLLAQEWPNSHIAKLCIKAASEAAERGDSQAARAVILAIMLAAENPNNEHAEECIRLAMEAARDGTSEHAERVERECWKHME

SEQ ID NO: 132 - protein, 148 aa, Artificial Sequence
  Synthetic polypeptide
DIEKLCKKAEEEAKEVGDNLAVAAVLTACLLAQEHPNADIAKLCIKAASEAAESGDKQAARAVILAIMLAAENPNADIAKLCIKAASEAAEGSKHGWSQAAEAVERAIWLAAENPNADIAKKCIKAASEAAREGNSQKAEEVKERCKS

SEQ ID NO: 133 - protein, 279 aa, Artificial Sequence
  Synthetic polypeptide
GSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGATNFSLLKQAGDVEENPGP

SEQ ID NO: 134 - DNA, 25 bp, Artificial Sequence
  Synthetic oligonucleotide
tctagagggtatataatgggggcca

SEQ ID NO: 135 - DNA, 97 bp, Homo sapiens
gaattcagggctgggcataaaagtcagggcagagccatctattgcttacatttgcttctgacacaactgtgttcactagcaacctcaaacagacacc

SEQ ID NO: 136 - DNA, 120 bp, Artificial Sequence
  Synthetic polynucleotide
tcgaggacattttgacacccccataatatttttccagaattaacagtataaattgcatctcttgttcaagagttccctatcactctctttaatcactactcacagtaacctcaactcctg

SEQ ID NO: 137 - DNA, 68 bp, Artificial Sequence
  Synthetic oligonucleotide
gaggtaggcgtgtacggtgggcgcctataaaagcagagctcgtttagtgaaccgtcagatcgcctgga

SEQ ID NO: 138 - DNA, 125 bp, Artificial Sequence
  Synthetic polynucleotide
acgtataagctttgcttatgtaaaccagggcgcctataaaagagtgctgattttttgagtaaacttcaattccacaacacttttgtcttataccaactttccgtaccacttcctaccctcgtaaa

SEQ ID NO: 139 - DNA, 386 bp, Artificial Sequence
  Synthetic polynucleotide
tgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggc

SEQ ID NO: 140 - DNA, 511 bp, Homo sapiens
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 141 - protein, 43 aa, Artificial Sequence
  Synthetic polypeptide
GSGATNFSLLKQAGDVEENPGPGSGEGRGSLLTCGDVEENPGP

SEQ ID NO: 142 - protein, 43 aa, Artificial Sequence
  Synthetic polypeptide
GSGEGRGSLLTCGDVEENPGPGSGATNFSLLKQAGDVEENPGP

SEQ ID NO: 143 - DNA, 528 bp, Artificial Sequence
  Synthetic polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagat

SEQ ID NO: 144 - DNA, 533 bp, Artificial Sequence
  Synthetic polynucleotide
attgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgatcgatt

SEQ ID NO: 145 - DNA, 408 bp, Artificial Sequence
  Synthetic polynucleotide
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 146 - DNA, 212 bp, Artificial Sequence
  Synthetic polynucleotide
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 147 - DNA, 1219 bp, Artificial Sequence
  Synthetic polynucleotide
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacatcgatt